(19) United States
(12) Patent Application Publication
(10) Pub. No.: US 2026/0285986 A1
(43) Pub. Date: Sep. 24, 2026

(54) MOLECULES THAT BIND TO CD135 POLYPEPTIDES

(71) Applicants: University of Pittsburgh - Of the Commonwealth System of Higher Education, Pittsburgh, PA (US); The Regents of the University of Colorado, Denver, CO (US)

(72) Inventors: Dimiter Stanchev Dimitrov, Pittsburgh, PA (US); Wei Li, Pittsburgh, PA (US); Terry James Fry, Boulder, CO (US); Michael Yarnell, Boulder, CO (US)

(21) Appl. No.: 19/108,354

(22) PCT Filed: Nov. 17, 2023
(86) PCT No.: PCT/US2023/080350

§ 371 Date: Mar. 3, 2025

Related U.S. Application Data
(60) Provisional application No. 63/426,474, filed on Nov. 18, 2022.

(51) Int. Cl. C07K 16/28; A61K 38/00; A61K 40/31; A61K 40/41; A61K 47/68; C07K 14/705

(52) U.S. Cl. CPC C07K 16/2863; A61K 40/31; A61K 40/41; A61K 47/6849; C07K 14/7051; A61K 38/00; C07K 2319/00

(57) ABSTRACT

This document provides methods and materials involved in binding a binder (e.g., an antibody, antigen binding fragment, antibody domain, CAR, cell engager, and/or ADC) to a CD135 polypeptide. For example, binders (e.g., antibodies, antigen binding fragments, antibody domains, CARs, cell engagers, and/or ADCs) that bind to a CD135 polypeptide and methods and materials for using one or more such binding molecules to treat a mammal (e.g., a human) having cancer are provided.

### CROSS-REFERENCE TO RELATED APPLICATIONS

This is a National Stage application under 35 U.S.C. § 371 of International Application No. PCT/US2023/080350, having an International Filing Date of Nov. 17, 2023, which application claims the benefit of U.S. Provisional Application Ser. No. 63/426,474, filed Nov. 18, 2022. The disclosure of the prior application is considered part of the disclosure of this application, and is incorporated in its entirety into this application.

### SEQUENCE LISTING

This application contains a Sequence Listing that has been submitted electronically as an XML file named 48881-0043WO1_SL.xml. The XML file, created on Jan. 17, 2024, is 331,191 bytes in size. The material in the XML file is hereby incorporated by reference in its entirety.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a CD135 polypeptide. For example, this document provides binders (e.g., antibodies, antigen binding fragments, antibody domains, CARs, cell engagers, or ADCs) that bind to a CD135 polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a CD135 polypeptide and methods and materials for using such cells to treat cancer.

### 2. Background Information

Fetal liver tyrosine kinase 3 (FLT-3; also known as cluster of differentiation antigen 135 (CD135)) is a single transmembrane protein with five immunoglobulin-like domains, a transmembrane domain, and an intracellular kinase domain. The extracellular domain is responsible for binding to its ligand (FLT3L), leading to the dimerization of the receptor and triggering kinase activity that can initiate downstream phosphorylation mediated signaling pathways such as the PI3 kinase/AKT pathway. The normal function of FLT3L/FLT3 involves the stimulation of proliferation of hematopoietic stem progenitor and dendritic cells.

Recently, the correlation of tandem duplication mutations and point mutations (e.g., D835 and 1836) in CD135 to the myeloid transformation was identified (Whitman et al., Blood, 111(3):1552-9 (2008)). It was reported that CD135 mutations are frequent mutations in acute myeloid leukemia (AML), which mutations constitutively activate the kinase activity of CD135, promoting cell proliferation and resistance to apoptosis (Grafone et al., Oncol. Rev., 6(1):e8 (2012)). Overall, CD135 is expressed in approximately 98% of pre-B acute lymphocytic leukemia (ALL) patients and in about 90% of AML patients (Terwilliger et al., Blood Cancer J., 7(6):e577 (2017)). The CD135 mutations in both adult and pediatric AML patients were associated with poor prognosis (Vela-Ojeda et al., Arch. Med. Res., 52(2):217-223 (2021)). Small molecule CD135 inhibitors such as midostaurin and sorafinib that can block CD135 kinase activity are being clinically developed (Kazi et al., Physiol. Rev., 99(3):1433-1466 (2019)). The anti-CD135 antibody named IMC-EB10 was reported to inhibit CD135 signaling, decrease the engraftment of primary AML CD135-ITD (CD135-internal tandem duplication) cells, and improve survival of NOD/SCID mice injected with human leukemia cells (Piloto et al., Cancer Res., 66(9):4843-51 (2006)).

### SUMMARY

This document provides methods and materials involved in binding a molecule (e.g., an antibody, an antigen binding fragment, an antibody domain, a CAR, a cell engager, or an ADC) to a CD135 polypeptide. For example, this document provides binders (e.g., antibodies, antigen binding fragments, antibody domains, CARs, cell engagers, or ADCs) that bind to a CD135 polypeptide and methods and materials for using one or more such binders to treat a mammal (e.g., a human) having cancer (e.g., leukemia such as acute myeloid leukemia (AML) and/or acute lymphocytic leukemia (ALL)).

This document also provides cells (e.g., host cells) designed to express one or more binders (e.g., antibodies, antigen binding fragments, antibody domains, CARs, or cell engagers) having the ability to bind to a CD135 polypeptide and methods and materials for using such cells to treat cancer (e.g., leukemia such as AML and/or ALL).

As described herein, binders (e.g., one or more antibodies, one or more antigen binding fragments, one or more antibody domains, one or more CARs, one or more cell engagers, and/or one or more ADCs) can be designed to have the ability to bind to a CD135 polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human CD135 polypeptide as set forth in SEQ ID NO:37 and/or SEQ ID NO:39 (see, e.g., FIG. 1).

As described herein, anti-CD135 binders (e.g., Clones #1, #2, #3, and #4) bind to CD135 polypeptides (e.g., human CD135 polypeptides). See, e.g., FIGS. 39, 40, 42, and 43. In addition, administration of human primary T cells designed to express CAR Clone #1A, CAR Clone #2A, CAR Clone #3A, or CAR Clone #4A to mammals with Molm14 leukemia prolonged survival as compared to mammals administered T cells designed to express anti-CD19 CARs (control; irrelevant CAR; 19_28z) or mock-transfected cells (FIGS. 47 and 53). These results demonstrate that anti-CD135 binders provided herein can be used to treat cancer (e.g., leukemia such as AML and/or ALL).

In some cases, a single set of three CDRs of an antibody domain (e.g., a VH domain) provided herein (e.g., SEQ ID NOs:1-3; SEQ ID NOs:9-11; SEQ ID NOs:17-19; or SEQ ID NOs:25-27) can be engineered into a CAR to create CAR⁺ cells (e.g., CAR⁺ T cells, CAR⁺ stem cells such as CAR⁺ induced pluripotent stem cells, or CAR⁺ NK cells) having the ability to target CD135⁺ cells (e.g., CD135⁺ tumor cells and/or CD135⁺ tumor vasculature), can be engineered into an antibody structure that includes an Fc region to create antibodies having the ability to target CD135⁺ cells (e.g., CD135⁺ tumor cells and/or CD135⁺ tumor vasculature) and induce ADCC against the target CD135⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target CD135⁺ cells (e.g., CD135⁺ tumor cells and/or CD135⁺ tumor vasculature) and induce one or more immune responses (e.g., T cell immune responses and/or ADCC using a cell engager in the absence of an Fc-containing antibody) against the target CD135⁺ cells. It is noted that BiKE- and TriKE-mediated killing can be referred to as ADCC even though it is not initiated by an Fc domain in cases when the engager lacks an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, scFv-drug conjugates, and/or antibody domain-drug conjugates can be designed to include an appropriate binder provided herein to create the conjugate. Such conjugates can be used to deliver the drug payload to target cells such as cancer cells (e.g., CD135⁺ cancer cells) or cancer vasculature (e.g., CD135⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a CD135⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer (e.g., leukemia such as AML and/or ALL).

As also described herein, cells (e.g., host cells) can be designed to express one or more binders (e.g., antibodies, antigen binding fragments, antibody domains, CARs, or cell engagers) having the ability to bind to a CD135 polypeptide. For example, cells such as T cells (e.g., CTLs), stem cells (e.g., induced pluripotent stem cells), or NK cells can be engineered to express one or more CARs having the ability to bind to a CD135 polypeptide. Such cells (e.g., CD135-specific CAR⁺ T cells or NK cells) can be used to treat cancer (e.g., leukemia such as AML and/or ALL).

In some cases, a binder (e.g., an antibody, antigen binding fragment, and/or antibody domain) provided herein can be used to detect the presence or absence of a CD135 polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains CD135⁺ cells (e.g., CD135⁺ cancer cells). Having the ability to detect the presence or absence of a CD135 polypeptide (e.g., CD135⁺ cancer cells) can allow clinicians, health professionals, and patients to make better decisions about possible treatment options. For example, detection of CD135⁺ cancer cells within a mammal can allow clinicians, health professionals, and patients to select an appropriate anti-cancer treatment that targets the CD135⁺ cancer cells. Such treatments that target the CD135⁺ cancer cells can include administration of one or more of the binders described herein having the ability to bind to a CD135 polypeptide and/or administration of one or more cells (e.g., CD135-specific CAR⁺ T cells or NK cells) designed to express a binder described herein.

In general, one aspect of this document features an antibody comprising (or consisting essentially of, or consisting of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); or (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:37 and/or SEQ ID NO:39. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the heavy chain variable domain or region of the (ii). The heavy chain variable domain or region can comprise an amino acid sequence having at least 90 percent identity to the amino acid sequence set forth in SEQ ID NO:16. The antibody can comprise the heavy chain variable domain or region of the (iii). The heavy chain variable domain or region can comprise an amino acid sequence having at least 90 percent identity to the amino acid sequence set forth in SEQ ID NO:24. The antibody can comprise the heavy chain variable domain or region of the (iv). The heavy chain variable domain or region can comprise an amino acid sequence having at least 90 percent identity to the amino acid sequence set forth in SEQ ID NO:32. The antibody can be a monoclonal antibody. The antibody can be an scFv antibody. This paragraph can be referred to as first aspect paragraph.

In another aspect, this document features an antigen binding fragment comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); or (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:37, and/or SEQ ID NO:39. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the heavy chain variable domain or region of the (ii). The heavy chain variable domain or region can comprise an amino acid sequence having at least 90 percent identity to the amino acid sequence set forth in SEQ ID NO:16. The antigen binding fragment can comprise the heavy chain variable domain or region of the (iii). The heavy chain variable domain or region can comprise an amino acid sequence having at least 90 percent identity to the amino acid sequence set forth in SEQ ID NO:24. The antigen binding fragment can comprise the heavy chain variable domain or region of the (iv). The heavy chain variable domain or region can comprise an amino acid sequence having at least 90 percent identity to the amino acid sequence set forth in SEQ ID NO:32. The antigen binding fragment can be monoclonal. The antigen binding fragment can be a Fab. This paragraph can be referred to as second aspect paragraph.

In another aspect, this document features an antibody domain comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); or (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody domain can comprise the ability to bind to SEQ ID NO:37 and/or SEQ ID NO:39. The antibody domain can comprise the heavy chain variable domain or region of the (i). The heavy chain variable domain or region can comprise an amino acid sequence having at least 90 percent identity to the amino acid sequence set forth in SEQ ID NO:8. The antibody domain can comprise the heavy chain variable domain or region of the (ii). The heavy chain variable domain or region can comprise an amino acid sequence having at least 90 percent identity to the amino acid sequence set forth in SEQ ID NO:16. The antibody domain can comprise the heavy chain variable domain or region of the (iii). The heavy chain variable domain or region can comprise an amino acid sequence having at least 90 percent identity to the amino acid sequence set forth in SEQ ID NO:24. The antibody domain can comprise the heavy chain variable domain or region of the (iv). The heavy chain variable domain or region can comprise an amino acid sequence having at least 90 percent identity to the amino acid sequence set forth in SEQ ID NO:32. The antibody domain can be monoclonal. The antibody domain can be a VH domain. This paragraph can be referred to as third aspect paragraph.

In another aspect, this document features a chimeric antigen receptor comprising (or consisting essentially of, or consisting of) an antigen binding domain, a hinge, a transmembrane domain, and one or more signaling domains, wherein the antigen binding domain comprises (or consists essentially of, or consists of) any antibody, any antigen-binding fragment, or any antibody domain of any of first aspect paragraph, second aspect paragraph, or third aspect paragraph. The antigen binding domain can comprise a VH domain having the ability to bind to a CD135 polypeptide. The hinge can comprise a hinge set forth in FIG. 17. The transmembrane domain can comprise a transmembrane domain set forth in FIG. 18. The chimeric antigen receptor can comprise one or more signaling domains set forth in FIG. 19. This paragraph can be referred to as fourth aspect paragraph.

In another aspect, this document features a cell comprising (or consisting essentially of, or consisting of) any chimeric antigen receptor of the preceding paragraph. The cell can be a T cell, a stem cell, or an NK cell.

In another aspect, this document features a cell engager comprising (or consisting essentially of, or consisting of) a first antigen binding domain, a linker, and a second antigen binding domain, wherein the first antigen binding domain comprises (or consists essentially of, or consists of) any antibody, any antigen-binding fragment, or any antibody domain of any of first aspect paragraph, second aspect paragraph, or third aspect paragraph. The first antigen binding domain can comprise a VH domain having the ability to bind to a CD135 polypeptide. The first antigen binding domain can be an IgG having the ability to bind to a CD135 polypeptide. The linker can comprise a linker set forth in FIG. 13 or FIG. 17. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 29. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 30. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 30. This paragraph can be referred to as fifth aspect paragraph.

In another aspect, this document features a nucleic acid comprising (or consisting essentially of, or consisting of) a nucleic acid sequence encoding at least part of the antibody, the antigen-binding fragment, or the antibody domain of any of first aspect paragraph, second aspect paragraph, or third aspect paragraph. The nucleic acid sequence can encode the heavy chain variable domain or region of any one of the (i)-(viii) of first aspect paragraph. The nucleic acid can be a viral vector. The nucleic acid can be a phagemid.

In another aspect, this document features a nucleic acid comprising (or consisting essentially of, or consisting of) a nucleic acid sequence encoding any chimeric antigen receptor of fourth aspect paragraph or any cell engager of fifth aspect paragraph. The nucleic acid can be a viral vector. The nucleic acid can be a phagemid.

In another aspect, this document features a host cell comprising (or consisting essentially of, or consisting of) any nucleic acid of the preceding paragraph. This paragraph can be referred to as sixth aspect paragraph.

In another aspect, this document features a host cell that expresses any chimeric antigen receptor of fourth aspect paragraph or any cell engager of fifth aspect paragraph. The host cell can be a T cell, stem cell, or NK cell. This paragraph can be referred to as seventh aspect paragraph.

In another aspect, this document features an antibody-drug conjugate (ADC) comprising (or consisting essentially of, or consisting of) an antigen binging domain covalently linked to a drug, wherein the antigen binging domain comprises an antibody, an antigen binding fragment, or an antibody domain of any of first aspect paragraph, second aspect paragraph, or third aspect paragraph. The antigen binding domain can comprise a VH domain having the ability to bind to a CD135 polypeptide. The drug can be selected from the group consisting of auristatins, mertansine, or pyrrolobenzodiazepine (PBD) dimers. This paragraph can be referred to as eighth aspect paragraph.

In another aspect, this document features a composition comprising (or consisting essentially of, or consisting of) an antibody, an antigen binding fragment, or an antibody domain of any of first aspect paragraph, second aspect paragraph, or third aspect paragraph. The composition can comprise any antibody of first aspect paragraph. The composition can comprise any antigen binding fragment of second aspect paragraph. The composition can comprise any antibody domain of third aspect paragraph.

In another aspect, this document features a composition comprising (or consisting essentially of, or consisting of) any cell engager of fifth aspect paragraph.

In another aspect, this document features a composition comprising (or consisting essentially of, or consisting of) any cell of sixth aspect paragraph or seventh aspect paragraph.

In another aspect, this document features a composition comprising (or consisting essentially of, or consisting of) any ADC of any one of eighth aspect paragraph. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of, or consists of) administering, to the mammal, a composition of any of the preceding four paragraphs. The mammal can be a human. The cancer can be a CD135⁺ cancer. The CD135⁺ cancer can be selected from the group consisting of CD135⁺ leukemias (e.g., CD135⁺ AMLs and/or CD135⁺ ALLs). The number of cancer cells within the mammal can be reduced following the administering step.

In another aspect, this document features a method of treating a mammal having cancer. The method comprises (or consists essentially of, or consists of) (a) administering, to the mammal, a composition of any of the preceding four paragraphs referred to in the preceding paragraph, and (b) administering, to the mammal, a composition comprising a checkpoint inhibitor. The mammal can be a human. The cancer can be a CD135⁺ cancer. The CD135⁺ cancer can be selected from the group consisting of CD135⁺ leukemias (e.g., CD135⁺ AAMLs and/or CD135⁺ ALLs). The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells within the mammal can be reduced following the administering steps (a) and (b).

In another aspect, this document features a method for binding a binding molecule to a CD135 polypeptide. The method comprises (or consists essentially of, or consists of) contacting the CD135 polypeptide with an antibody, an antigen binding fragment, or an antibody domain of any of first aspect paragraph, second aspect paragraph, or third aspect paragraph. The contacting can be performed in vitro. The contacting can be performed in vivo. The contacting can be performed within a mammal by administering the antibody, the antigen binding fragment, or the antibody domain to the mammal. The mammal can be a human.

In another aspect, this document features a method for binding a binding molecule to a CD135 polypeptide. The method comprises (or consists essentially of, or consists of) contacting the CD135 polypeptide with any chimeric antigen receptor of fourth aspect paragraph, any cell engager of fifth aspect paragraph, or any ADC of eighth aspect paragraph. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the amino acid sequences of an exemplary full length human CD135 polypeptide (SEQ ID NO:37) and an exemplary extracellular domain of a human CD135 polypeptide (SEQ ID NO:39), and exemplary nucleic acid sequences encoding each (SEQ ID NOs:38 and 40). Polypeptides consisting of SEQ ID NO:39 were used to identify Clone #1 and Clone #3.

FIG. 2 depicts the amino acid sequence of a VH domain designated Clone #1. The CDRs and framework sequences also are delineated.

FIG. 3 depicts the amino acid sequence of a VH domain designated Clone #2. The CDRs and framework sequences also are delineated.

FIG. 4 depicts the amino acid sequence of a VH domain designated Clone #3. The CDRs and framework sequences also are delineated.

FIG. 5 depicts the amino acid sequence of a VH domain designated Clone #4. The CDRs and framework sequences also are delineated.

FIG. 6 depicts the nucleic acid sequences encoding the indicated VH domains of Clones #1-#4.

FIG. 7 depicts the structure of an exemplary Ig and provides the amino acid and nucleic acid sequences of exemplary hinge, CH2, and CH3 regions/domains.

FIG. 8 depicts the structure of exemplary scFv's.

FIGS. 9A and 9B depict the amino acid sequences of an exemplary heavy chain variable domain (FIG. 9A) and an exemplary light chain variable domain (FIG. 9B) of an exemplary scFv. The CDRs and framework sequences of each also are delineated. An exemplary linker amino acid sequence such as a linker amino acid sequence set forth in FIG. 13 can be used to link the heavy chain variable domain and the light chain variable domain together to form a scFv.

FIGS. 10A and 10B depict the amino acid sequences of an exemplary heavy chain variable domain (FIG. 10A) and an exemplary light chain variable domain (FIG. 10B) of an exemplary scFv. The CDRs and framework sequences of each also are delineated. An exemplary linker amino acid sequence such as a linker amino acid sequence set forth in FIG. 13 can be used to link the heavy chain variable domain and the light chain variable domain together to form a scFv.

FIGS. 11A and 11B depict the amino acid sequences of an exemplary heavy chain variable domain (FIG. 11A) and an exemplary light chain variable domain (FIG. 11B) of an exemplary scFv. The CDRs and framework sequences of each also are delineated. An exemplary linker amino acid sequence such as a linker amino acid sequence set forth in FIG. 13 can be used to link the heavy chain variable domain and the light chain variable domain together to form a scFv.

FIGS. 12A and 12B depict the amino acid sequences of an exemplary heavy chain variable domain (FIG. 12A) and an exemplary light chain variable domain (FIG. 12B) of an exemplary scFv. The CDRs and framework sequences of each also are delineated. An exemplary linker amino acid sequence such as a linker amino acid sequence set forth in FIG. 13 can be used to link the heavy chain variable domain and the light chain variable domain together to form a scFv.

FIG. 13 depicts exemplary linker amino acid sequences that can be used to link a heavy chain variable domain and a light chain variable domain together to form a scFv. These linker sequences also can be used to create CARs and cell engagers.

FIG. 14A depicts the structure of exemplary CARs. FIG. 14B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein, followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 15A depicts the structure of exemplary CARs. FIG. 15B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a VH domain provided herein (e.g., a VH domain designed to include a set of three CDRs such as CDR1, CDR2, and CDR3 of a VH domain provided herein, for example, SEQ ID NOs:1-3; SEQ ID NOs:9-11; SEQ ID NOs:17-19; or SEQ ID NOs:25-27), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 16 depicts the amino acid sequences of exemplary signal peptides that can be used to design a CAR.

FIG. 17 depicts the amino acid sequences of exemplary hinges that can be used to design a CAR.

FIG. 18 depicts the amino acid sequences of exemplary transmembrane domains that can be used to design a CAR.

FIG. 19 depicts the amino acid sequences of exemplary intracellular signaling domains that can be used to design a CAR.

FIG. 20A depicts an amino acid sequence of an exemplary CAR (CAR Clone #1A) designed to include a VH domain of Clone #1, and FIG. 20B depicts a nucleic acid sequence encoding that CAR. The various components of this CAR (e.g., domains and linkers) are provided and delineated.

FIG. 21A depicts an amino acid sequence of a CAR (CAR Clone #2A) designed to include a VH domain of Clone #2, and FIG. 21B depicts a nucleic acid sequence encoding that CAR. The various components of this CAR (e.g., domains and linkers) are provided and delineated.

FIG. 22A depicts an amino acid sequence of a CAR (CAR Clone #3A) designed to include a VH domain of Clone #3, and FIG. 22B depicts a nucleic acid sequence encoding that CAR. The various components of this CAR (e.g., domains and linkers) are provided and delineated.

FIG. 23A depicts an amino acid sequence of a CAR (CAR Clone #4A) designed to include a VH domain of Clone #4, and FIG. 23B depicts a nucleic acid sequence encoding that CAR. The various components of this CAR (e.g., domains and linkers) are provided and delineated.

FIG. 24A depicts an amino acid sequence of an exemplary CAR (CAR Clone #1B designed to include a VH domain of Clone #1, and FIG. 24B depicts a nucleic acid sequence encoding that CAR. The various components of this CAR (e.g., domains and linkers) are provided and delineated.

FIG. 25A depicts an amino acid sequence of a CAR (CAR Clone #2B) designed to include a VH domain of Clone #2, and FIG. 25B depicts a nucleic acid sequence encoding that CAR. The various components of this CAR (e.g., domains and linkers) are provided and delineated.

FIG. 26A depicts an amino acid sequence of a CAR (CAR Clone #3B) designed to include a VH domain of Clone #3, and FIG. 26B depicts a nucleic acid sequence encoding that CAR. The various components of this CAR (e.g., domains and linkers) are provided and delineated.

FIG. 27A depicts an amino acid sequence of a CAR (CAR Clone #4B) designed to include a VH domain of Clone #4, and FIG. 27B depicts a nucleic acid sequence encoding that CAR. The various components of this CAR (e.g., domains and linkers) are provided and delineated.

FIG. 28A is a schematic of an exemplary BiTE designed using CDR1, CDR2, and CDR3 of a heavy chain and CDR1, CDR2, and CDR3 of a light chain in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G₄S)₃ linker (SEQ ID NO: 370)). FIG. 28B depicts an amino acid sequence of a linker sequence (SEQ ID NO:71; nucleic acid sequence of the linker is underlined in SEQ ID NO:219) followed by an gOKT3-7 scFv sequence, which can be attached to a light chain as shown in FIG. 28A. FIG. 28B also depicts a nucleic acid sequence encoding that linker and gOKT3-7 scFv.

FIG. 29 depicts the amino acid sequences of exemplary antigen binding domains that can be used to design cell engagers that bind to T cells.

FIG. 30 depicts the amino acid sequences of exemplary antigen binding domains that can be used to design cell engagers that bind to NK cells.

FIG. 31 depicts the amino acid sequence for an exemplary BiKE (BiKE #1) designed to include a VH domain of Clone #1. The various components of this BiKE (e.g., domains and linkers) are provided and delineated.

FIG. 32 depicts the amino acid sequence for an exemplary BiKE (BiKE #2) designed to include a VH domain of Clone #2. The various components of this BiKE (e.g., domains and linkers) are provided and delineated.

FIG. 33 depicts the amino acid sequence for an exemplary BiKE (BiKE #3) designed to include a VH domain of Clone #3. The various components of this BiKE (e.g., domains and linkers) are provided and delineated.

FIG. 34 depicts the amino acid sequence for an exemplary BiKE (BiKE #4) designed to include a VH domain of Clone #4. The various components of this BiKE (e.g., domains and linkers) are provided and delineated.

FIG. 35 depicts an amino acid sequence of a BiTE (BiTE #1) designed to include a VH domain of Clone #1 and a nucleic acid sequence encoding that BiTE. The various components of this BiTE (e.g., domains and linkers) are provided and delineated.

FIG. 36 depicts an amino acid sequence of a BiTE (BiTE #2) designed to include a VH domain of Clone #2 and a nucleic acid sequence encoding that BiTE. The various components of this BiTE (e.g., domains and linkers) are provided and delineated.

FIG. 37 depicts an amino acid sequence of a BiTE (BiTE #3) designed to include a VH domain of Clone #3 and a nucleic acid sequence encoding that BiTE. The various components of this BiTE (e.g., domains and linkers) are provided and delineated.

FIG. 38 depicts an amino acid sequence of a BiTE (BiTE #4) designed to include a VH domain of Clone #4 and a nucleic acid sequence encoding that BiTE. The various components of this BiTE (e.g., domains and linkers) are provided and delineated.

FIG. 39 is a graph plotting the binding of binders containing a C-terminal FLAG tag to an extracellular domain of a human CD135 polypeptide as determined by ELISA. 200 ng of an extracellular domain of a human CD135 polypeptide was coated onto immune-absorb plates, and the indicated concentrations of the indicated binders were added. After incubation and washing, the horse radish peroxidase (HRP)-conjugated anti-FLAG tag antibody was added to colorimetric development. 2G5 is Clone #3 (also referred to as G5); 2G5-F7 is Clone #4 (also referred to as G5-F7 or F7); H5 is Clone #1; H5-A10 is Clone #2; EB10 is an antibody with the epitope located at D4; and NC7 is an antibody with the epitope located at D5.

FIG. 40 is a graph plotting binding (mean fluorescence intensity; MFI) of the indicated binders containing a C-terminal FLAG tag to cell surface CD135 on RS4;11 human leukemia cell line cells. After incubation and washing, the PE-conjugated anti-FLAG tag antibody was used to detect binding by flow cytometry. 2G5 is Clone #3; 2G5-F7 is Clone #4; H5 is Clone #1; and H5-A10 is Clone #2.

FIG. 41 is a schematic of CD135 bound to it ligand (i.e., FLT3L) based on crystal structure data. Domains 1-5 are indicated as D1, D2, D3, D4, and D5, respectively.

FIG. 42 contains graphs plotting binding of the indicated binders to D1 of a human CD135 (top), to D2-D3 of CD135 (middle), and to D4-D5 of CD135 (bottom). 2G5 is Clone #3; 2G5-F7 is Clone #4; H5 is Clone #1; H5-A10 is Clone #2; EB10 is an antibody with the epitope located at D4; and NC7 is an antibody with the epitope located at D5.

FIG. 43 contains graphs plotting binding of the indicated binders to D2-D4 of a human CD135 (top) and to D1-D4 of CD135 (bottom). 2G5 is Clone #3; 2G5-F7 is Clone #4; H5 is Clone #1; H5-A10 is Clone #2; EB10 is an antibody with the epitope located at D4; and NC7 is an antibody with the epitope located at D5. From the results of FIGS. 42 and 43, Clones #1 and #2 were determined to bind to a conformational epitope in proximity to D5, which also depends on other domains, while Clones #3 and #4 bind to D1.

FIG. 44 is a graph plotting interferon-γ (IFN-γ) release from co-cultures of the indicated AML cell line cells (e.g., SEM, MV4-11, MOLM-14, or CMS cells) with T cells from a donor (donor D007) engineered to express the indicated CAR after 16 hours. IFN-γ release was measured by ELISA. SEM cells express high levels of CD135; MV4-11 express low levels of CD135; MOLM-14 express medium levels of CD135; and CMS cells do not express CD135. Mock is T cells not engineered to express a CAR; positive control CAR is NC7 scFv based CAR.

FIG. 45 is a graph plotting interleukin-2 (IL-2) release from co-cultures of the indicated AML cell line cells (e.g., SEM, MV4-11, MOLM-14, or CMS cells) with T cells engineered to express the indicated CAR after 16 hours. IFN-γ release was measured by ELISA. SEM cells express high levels of CD135; MV4-11 express low levels of CD135; MOLM-14 express medium levels of CD135; and CMS cells do not express CD135. Mock is T cells not engineered to express a CAR; positive control CAR is NC7 scFv based CAR.

FIG. 46 is a graph plotting tumor cell killing in co-cultures (1:1 E:T ratio) of the indicated GFP⁺ AAML cell line cells (e.g., SEM, MV4-11, MOLM-14, or CMS cells) with T cells engineered to express the indicated CAR after 36 hours. Tumor cell killing was measured by flow cytometry and loss of GFP⁺ tumor cells. SEM cells express high levels of CD135; MV4-11 express low levels of CD135; MOLM-14 express medium levels of CD135; and CMS cells do not express CD135. Mock is Tcells not engineered to express a CAR; positive control CAR is NC7 scFv based CAR. The results from FIGS. 44-46 demonstrate that antigen-specific (CD135-specific) cytokine production and tumor cell killing by the T cells engineered to express the CARs provided herein. The degree of activation and cytokine production appears to correlate with the density of CD135 expression on the target cancer cells.

FIG. 47 depicts in vivo data showing the anti-tumor activity of T cells (3×10⁶ cells) engineered to express the CARs provided herein as compared to the ineffective anti-tumor activity of T cells engineered to express CD19_28z CARs in mice administered 1×10⁶ Molm14 leukemia cells. Anti-tumor activity was determined by monitoring cancer cell growth/clearance by in vivo bioluminescent imaging. The CD19_28z CAR is an anti-CD19 CAR with a CD28 intracellular signaling domain. Administration of T cells designed to express any one of the four indicated CARs improved survival with CAR Clone #4A resulting in complete and sustained clearance of the Molm14 leukemia cancer.

FIG. 48 is a graph plotting Flux (photons/cm²/second) for the mice of FIG. 47. Data is presented as mean±standard deviation.

FIG. 49 is a graph plotting Kaplan-Meyer survival curves for the mice of FIG. 47.

FIG. 50 is a graph plotting interferon-γ (IFN-γ) release from co-cultures of the indicated AML cell line cells (e.g., SEM, MV4-11, MOLM-14, or CMS cells) with T cells from a donor (donor D013) engineered to express the indicated CAR after 16 hours. IFN-γ release was measured by ELISA. SEM cells express high levels of CD135; MV4-11 express low levels of CD135; MOLM-14 express medium levels of CD135; and CMS cells do not express CD135. Mock is T cells not engineered to express a CAR; positive control CAR is NC7 scFv based CAR.

FIG. 51 is a graph plotting interleukin-2 (IL-2) release from co-cultures of the indicated AML cell line cells (e.g., SEM, MV4-11, MOLM-14, or CMS cells) with T cells (from a different donor than the donor of FIG. 45) engineered to express the indicated CAR after 16 hours. IFN-γ release was measured by ELISA. SEM cells express high levels of CD135; MV4-11 express low levels of CD135; MOLM-14 express medium levels of CD135; and CMS cells do not express CD135. Mock is T cells not engineered to express a CAR; positive control CAR is NC7 scFv based CAR.

FIG. 52 is a graph plotting tumor cell killing in co-cultures (1:1 E:T ratio) of the indicated GFP⁺ AAML cell line cells (e.g., SEM, MV4-11, MOLM-14, or CMS cells) with T cells (from a different donor than the donor of FIG. 46) engineered to express the indicated CAR after 36 hours. Tumor cell killing was measured by flow cytometry and loss of GFP⁺ tumor cells. SEM cells express high levels of CD135; MV4-11 express low levels of CD135; MOLM-14 express medium levels of CD135; and CMS cells do not express CD135. Mock is T cells not engineered to express a CAR; positive control CAR is NC7 scFv based CAR. The results from FIGS. 50-52 demonstrate that antigen-specific (CD135-specific) cytokine production and tumor cell killing by the T cells engineered to express the CARs provided herein. The degree of activation and cytokine production appears to correlate with the density of CD135 expression on the target cancer cells.

FIG. 53 depicts in vivo data showing the anti-tumor activity of T cells (3×10⁶ cells; from a different donor than the donor of FIG. 47) engineered to express the CARs provided herein as compared to the ineffective anti-tumor activity of T cells (2×10⁶ cells) not engineered to express any CARs in mice administered 1×10⁶ Molm14 leukemia cell line cells. Anti-tumor activity was determined by monitoring cancer cell growth/clearance by in vivo bioluminescent imaging. Administration of T cells designed to express any one of the four indicated CARs improved survival with CAR Clone #4A resulting in complete and sustained clearance of the Molm14 leukemia cancer.

FIG. 54 is a graph plotting Flux (photons/cm²/second) for the mice of FIG. 53. Data is presented as mean±standard deviation.

FIG. 55 is a graph plotting Kaplan-Meyer survival curves for the mice of FIG. 53.

FIGS. 56A-B. Flt3 targeting biparatopic CARs. FIG. 56A: Individual heavy chain only binders against discrete epitopes on Flt3 were chosen to be combined into a single biparatopic binder. FIG. 56B: Individual VH binders were joined with a flexible intermolecular linker (G4S×4 (SEQ ID NO: 379)) and used in place of an scFV/VH on a second generation 4-1BB costimulated CAR. Multiple combinations and orientations were generated to determine effective formats.

FIGS. 57A-B. In Vitro antitumor activity of Flt3 targeting heavy chain only and biparatopic CARs against various cell lines. FIG. 57A: IL-2 production after 24 hours in vitro coculture, measured by ELISA. FIG. 57B: Cytotoxic lysis after 24 hours in vitro coculture at a 1:1 E:T ratio as measured by Flow Cytometry and loss of GFP⁺ tumor cells (lower bars indicate increased killing). All eights CARs exhibited antigen specific cytokine production and killing. Degree of activation and cytokine production appeared to be related to density of antigen expression on the target cell lines and is not strictly additive in the case of the biparatopic CARs. SEM=Flt3 high; Molm14=Flt3 medium; and Nalm6=Flt3 negative. Error bars are ±1 standard deviation.

FIGS. 58A-C. In Vivo antitumor activity of Flt3 targeting heavy chain only and biparatopic CARs against Molm14 leukemia. FIG. 58A: Growth/clearance of the tumor as monitored by in vivo bioluminescent imaging. FIG. 58B: Tumor burden graphed as Flux (photons/cm²/second), data displayed as mean±SD. FIG. 58C: Kaplan-Meyer survival curve. Multiple CARs exhibited anti-tumor activity in vivo and significantly improve survival. H5-G5 biparatopic exhibited complete and sustained clearance of the tumor in 60 percent of the mice.

### DETAILED DESCRIPTION

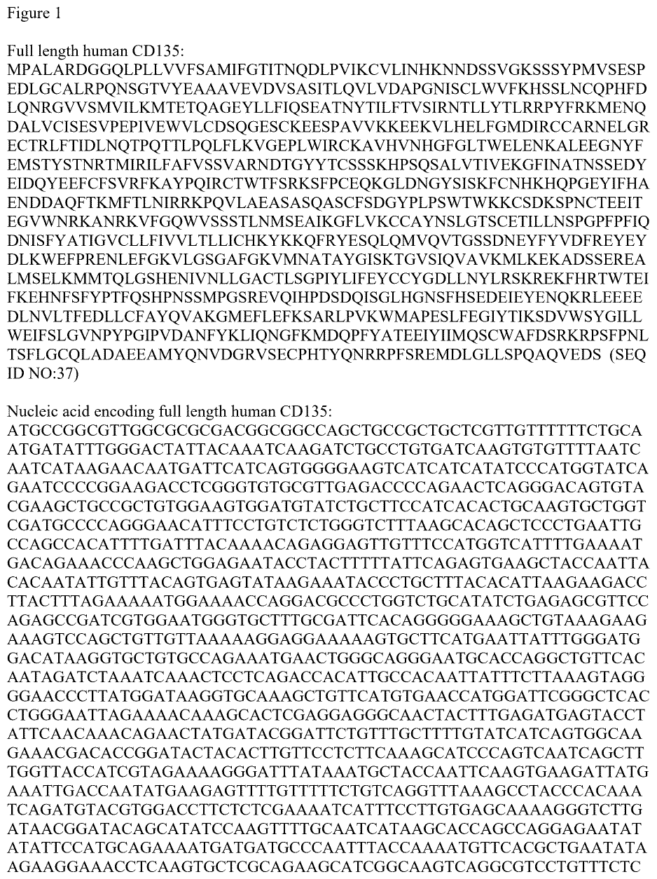

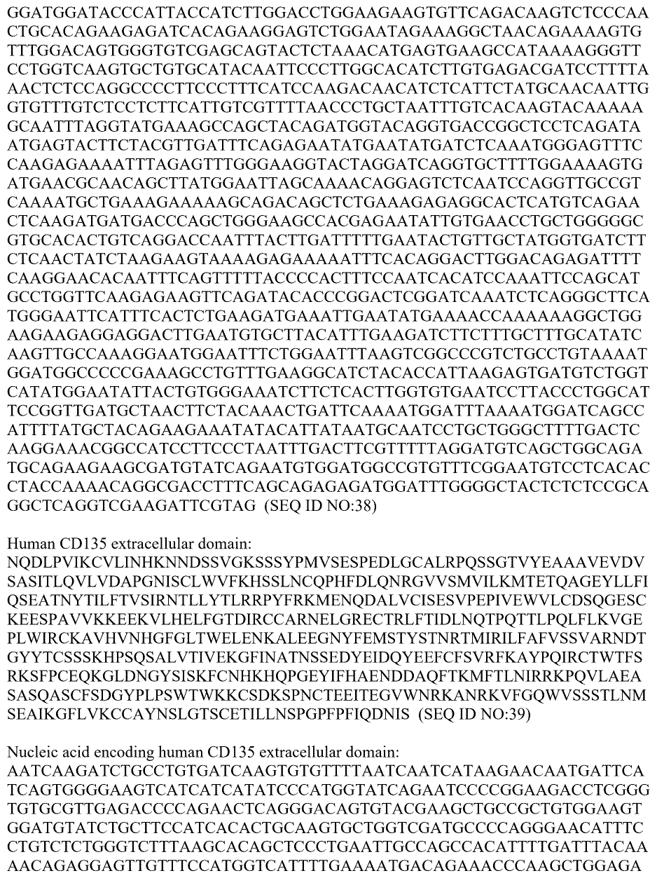

This document provides binders (e.g., antibodies, antigen binding fragments, antibody domains, CARs, cell engagers, and ADCs) that bind (e.g., specifically bind) to a CD135 polypeptide (e.g., a human CD135 polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:37 and/or SEQ ID NO:39 (see, e.g., FIG. 1).

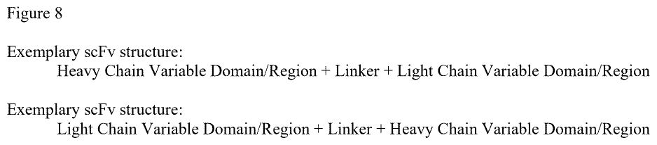

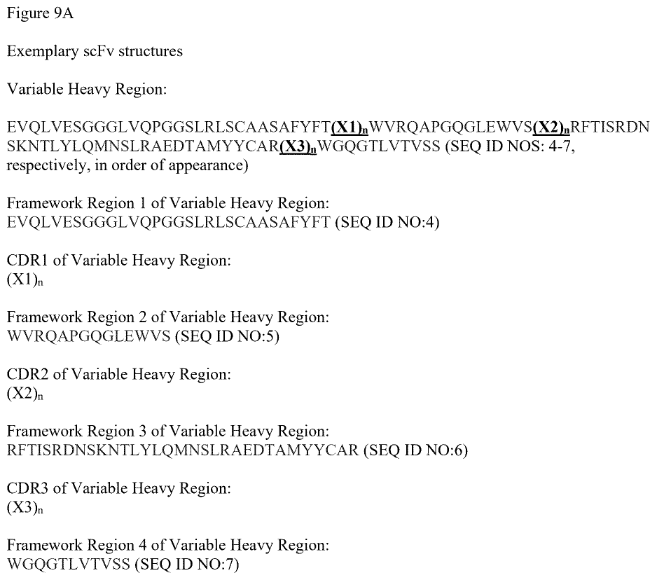

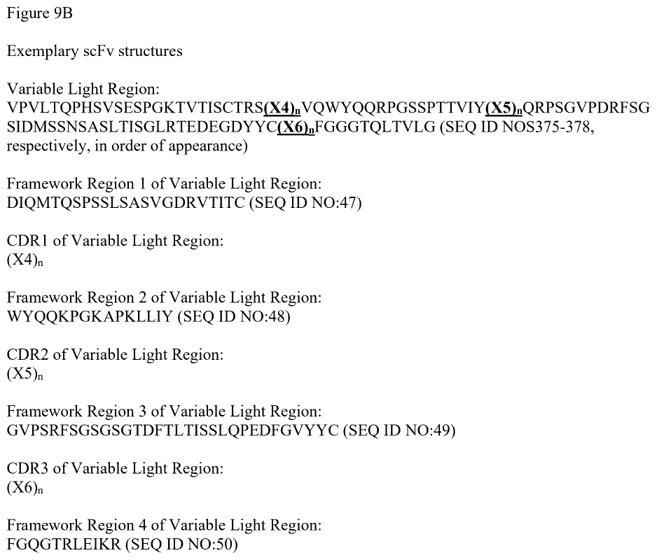

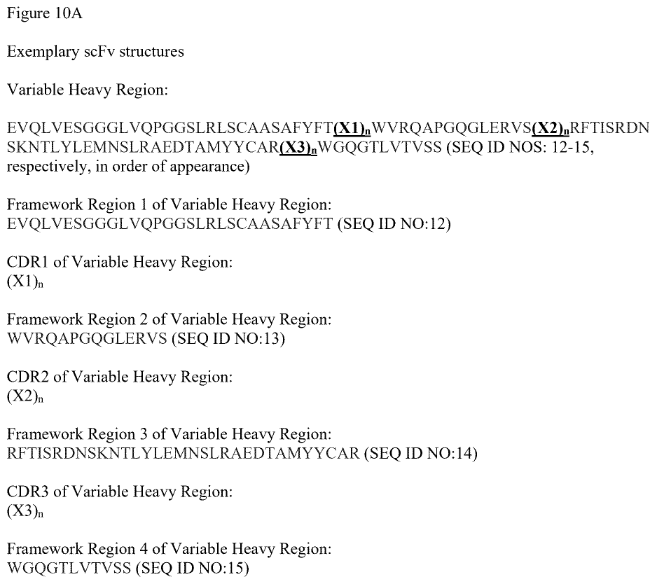

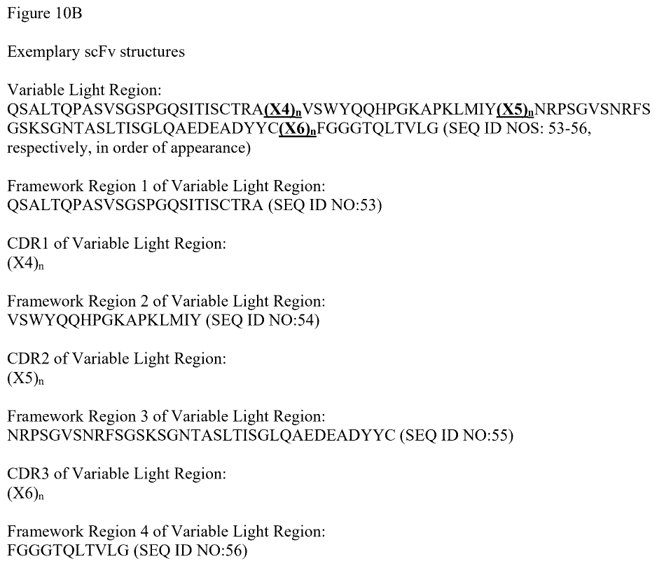

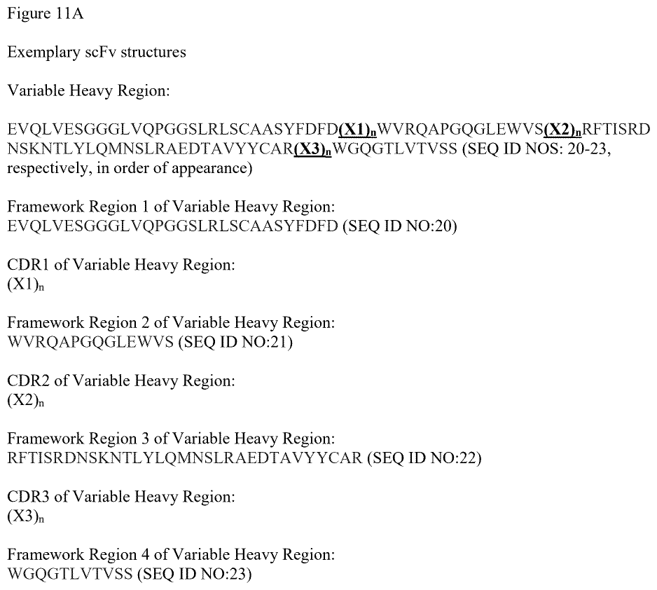

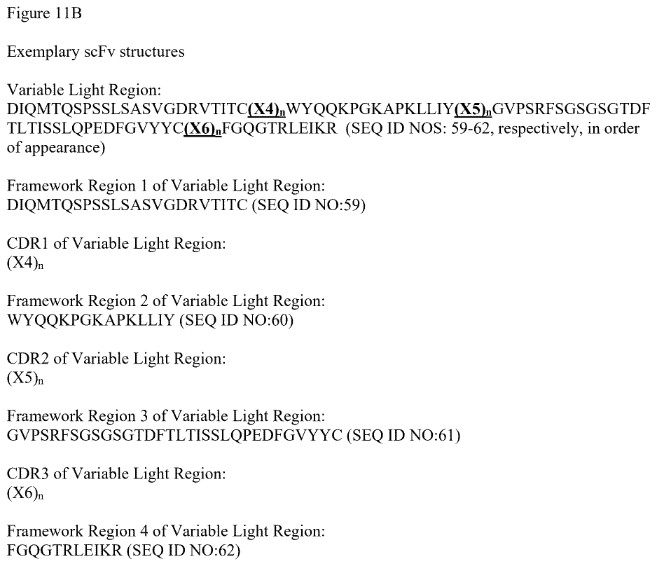

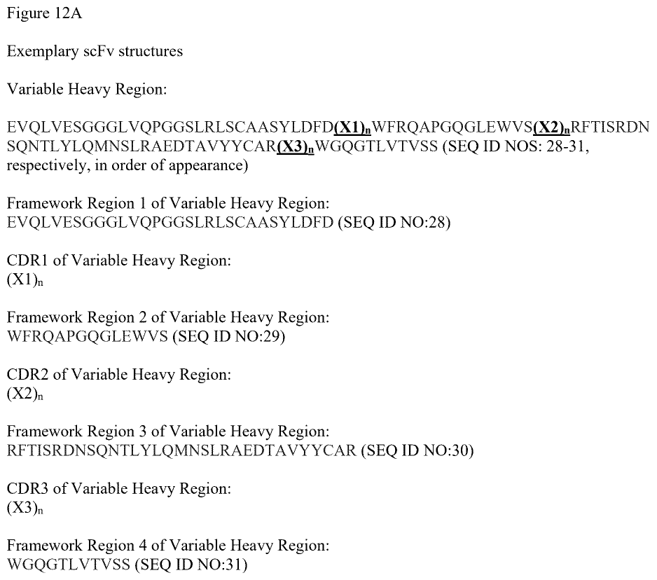

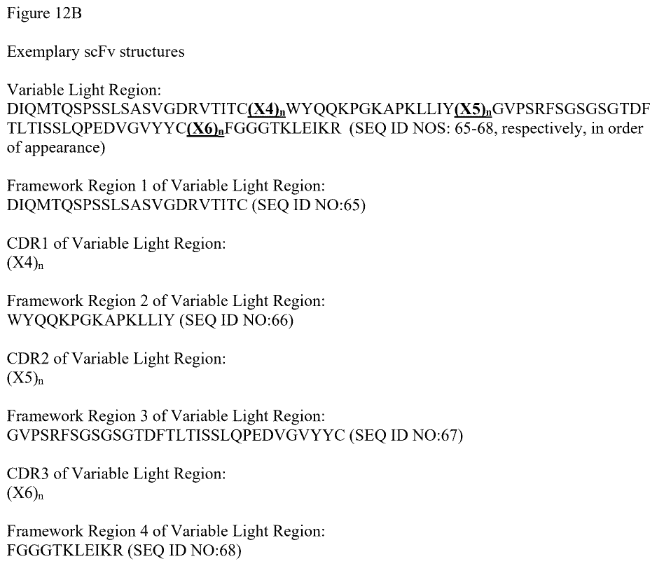

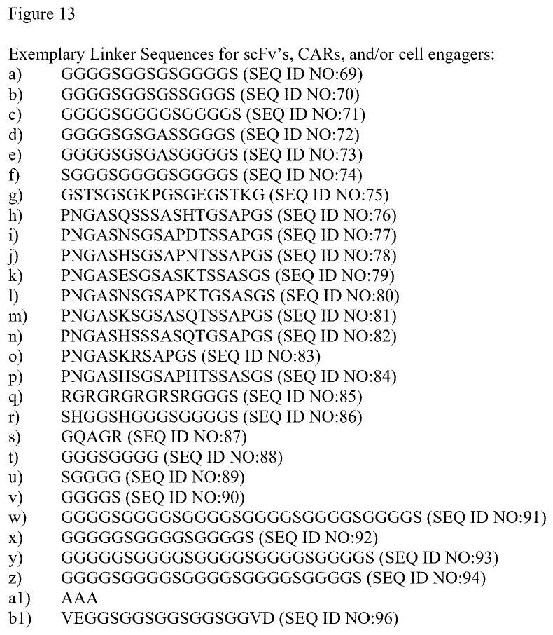

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 13). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 13). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 8, 9A-9B, 10A-10B, 11A-11B, and 12A-12B.

An antibody provided herein can include the CDRs as described herein (e.g., as described in Table 13) and can be configured to be a human antibody, a humanized antibody, or a chimeric antibody. In some cases, an antibody provided herein can include the CDRs as described herein (e.g., as described in Table 13) and can be a monoclonal antibody. In some cases, an antibody provided herein can include the CDRs as described herein (e.g., as described in Table 13) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 13) and can be configured to be a human antigen binding fragment, a humanized antigen binding fragment, or a chimeric antigen binding fragment. In some cases, an antigen binding fragment provided herein can include the CDRs as described herein (e.g., as described in Table 13) and can be a monoclonal antigen binding fragment. In some cases, an antigen binding fragment provided herein can include the CDRs as described herein (e.g., as described in Table 13) and can be configured as a Fab antibody. In some cases, a Fab antibody can include a partial hinge sequence (e.g., EPKSCDKT (SEQ ID NO:248)) for disulfide bonding between heavy and light chains of the Fab.

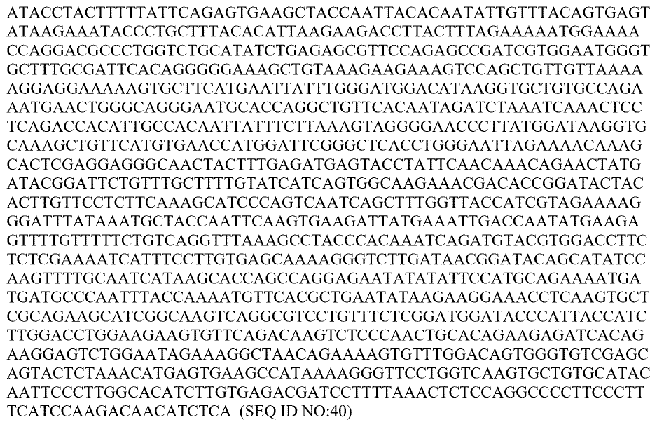

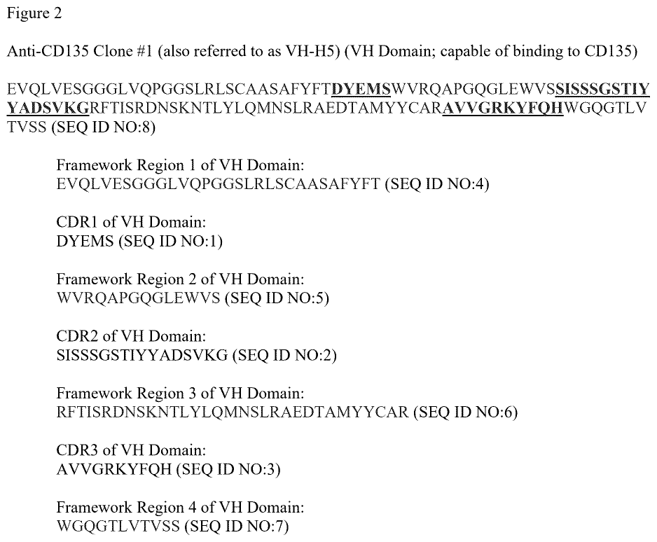

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 13) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 13) and can be a monoclonal antibody domain. In some cases, an antibody domain provided herein can include the CDRs as described herein (e.g., as described in Table 13) and can be engineered as a single VH domain or a single VL domain. Examples of VH domain's provided herein include, without limitation, those structures set forth in FIGS. 2-5.

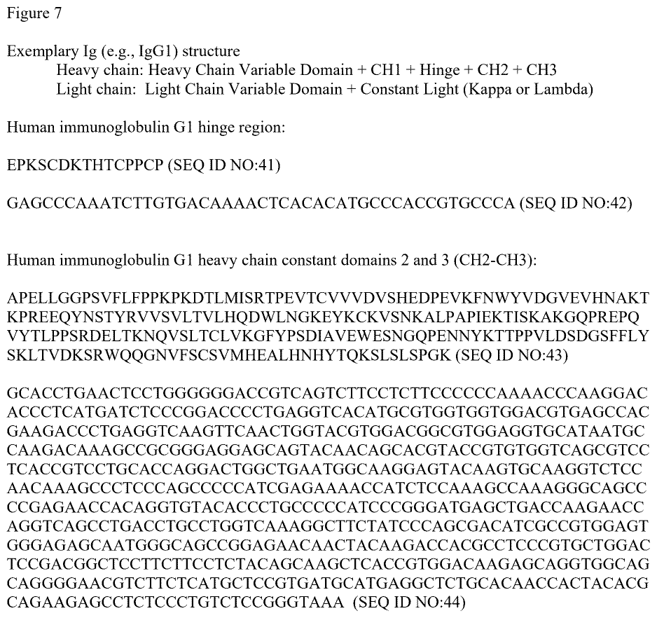

An anti-CD135 antibody, anti-CD135 antigen binding fragment, or anti-CD135 antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-CD135 antibody) can be a scFv antibody. In some cases, an antigen binding fragment provided herein (e.g., an anti-CD135 antibody fragment) can be a Fab. In some cases, an antibody provided herein (e.g., an anti-CD135 antibody) can be a fully intact antibody having the structure set forth in FIG. 7. In some cases, an antibody domain provided herein (e.g., an anti-CD135 antibody domain) can be a VH domain.

In some cases, an anti-CD135 antibody, anti-CD135 antigen binding fragment, or anti-CD135 antibody domain provided herein can be fully human. In some cases, an anti-CD135 antibody, anti-CD135 antigen binding fragment, or anti-CD135 antibody domain provided herein can have a low risk for inducing immunogenicity within a human.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a CD135 polypeptide (e.g., a human CD135 polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a CD135 polypeptide (e.g., a human CD135 polypeptide). In some examples, a CAR provided herein can be designed to include an antigen binding domain that includes a single set of three CDRs (e.g., a CDR1, CDR2, and CDR3) of an antibody domain (e.g., a VH domain) provided herein (e.g., SEQ ID NOs:1-3; SEQ ID NOs:9-11; SEQ ID NOs:17-19; or SEQ ID NOs:25-27). In some cases, an antigen binding domain of a CAR targeting a CD135 polypeptide can be designed to include a VH domain described herein or a scFv antibody described herein.

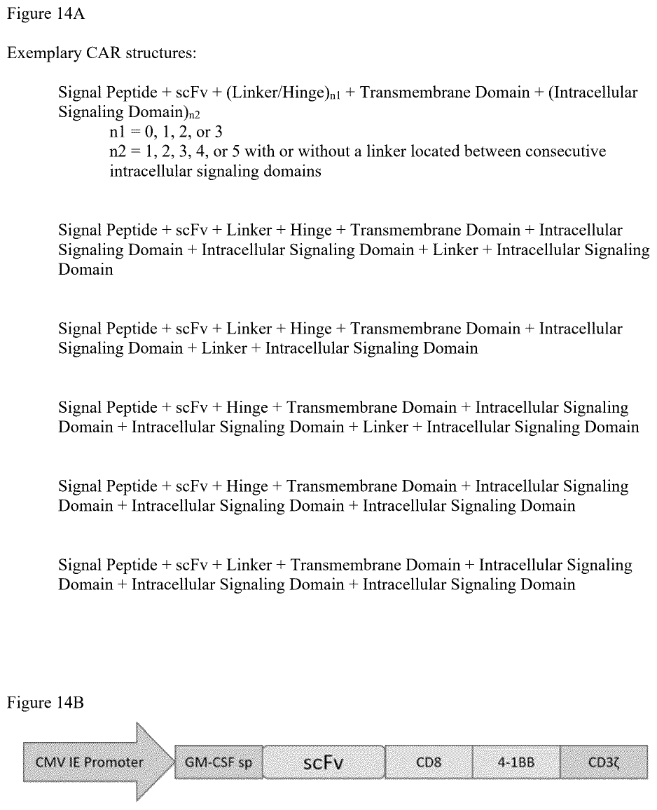

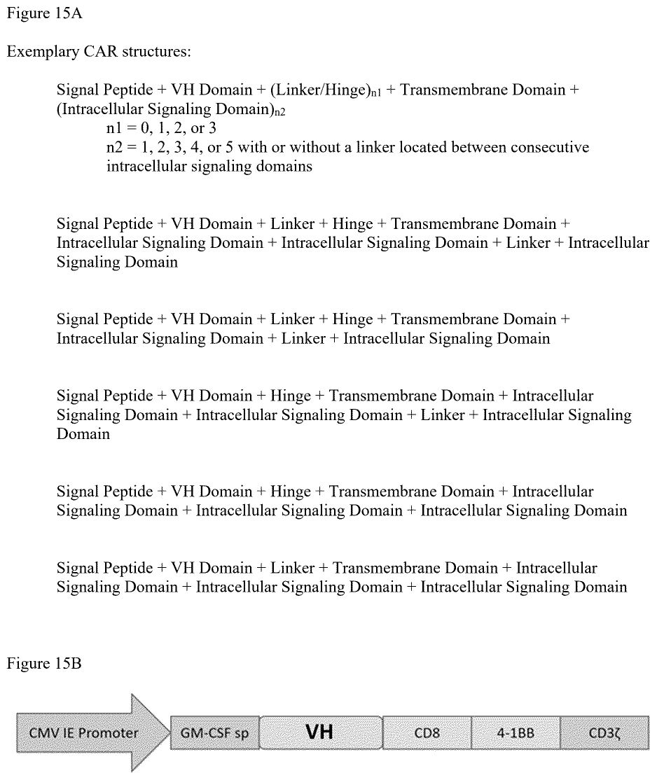

Examples of CAR structures that can be used to make a CAR provided herein include, without limitation, those set forth in FIGS. 14A-14B, 15A-15B, 20A-20B, 21A-21B, 22A-22B, 23A-23B, 24A-24B, 25A-25B, 26A-26B, and 27A-27B.

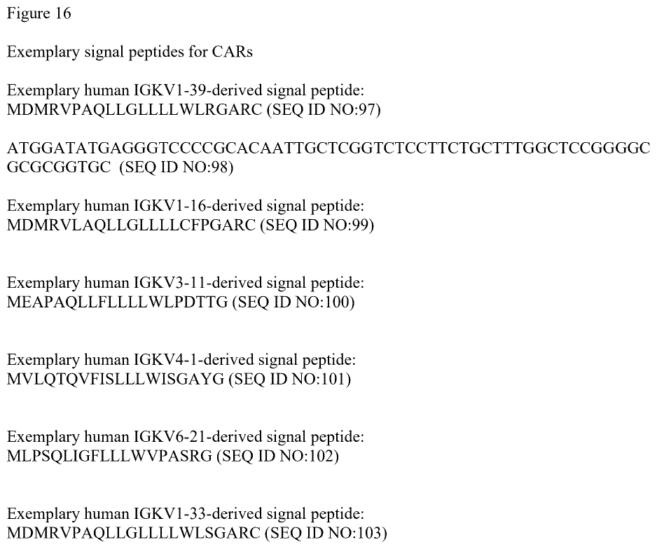

In some cases, a CAR provided herein can be designed to include a signal peptide. Any appropriate signal peptide can be used to design a CAR described herein. Examples of signal peptide that can be used to make a CAR described herein include without limitation, a human IGKV1-39-derived signal peptide, IGKV1-16, IGKV1-33, IGKV3-11, IGKV4-1, or IGKV6-21. In some cases, a CAR provided herein can be designed to include a signal peptide that comprises, consists essentially of, or consists of one of the amino acid sequences set forth in FIG. 16. In some cases, a CAR provided herein can be designed to include a signal peptide that comprises, consists essentially of, or consists of one of the amino acid sequences set forth in FIG. 16 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in FIG. 16 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in FIG. 13 can be used as a hinge of a CAR described herein.

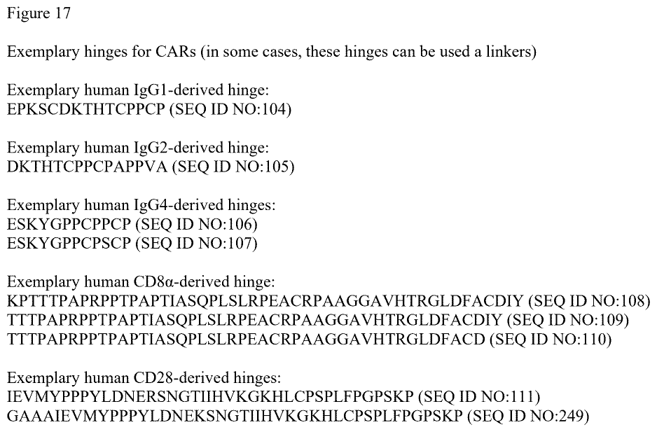

In some cases, a CAR provided herein can be designed to include a hinge that comprises, consists essentially of, or consists of one of the amino acid sequences set forth in FIG. 13 or FIG. 17. In some cases, a CAR provided herein can be designed to include a hinge that comprises, consists essentially of, or consists of one of the amino acid sequences set forth in FIG. 13 or FIG. 17 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 13 or FIG. 17 with two or less, three or less, four or less, five or less, six or less, seven or less, eight or less, nine or less, or ten or less amino acid deletions, additions, substitutions, or combinations thereof.

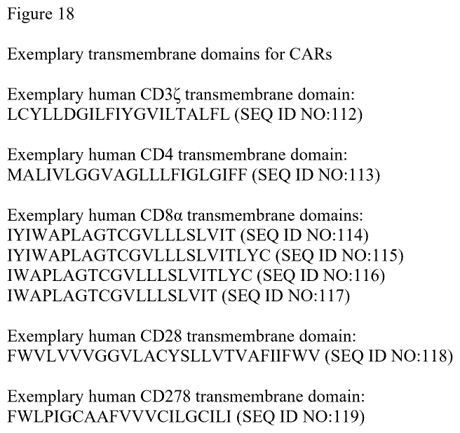

A CAR provided herein can be designed to include any appropriate transmembrane domain. For example, the transmembrane domain of a CAR provided herein can be, without limitation, a CD3ζ transmembrane domain, a CD4 transmembrane domain, a CD8a transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 18. In some cases, a CAR provided herein can be designed to include a transmembrane domain that comprises, consists essentially of, or consists of one of the amino acid sequences set forth in FIG. 18 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 18 with two or less, three or less, four or less, five or less, six or less, seven or less, eight or less, nine or less, or ten or less amino acid deletions, additions, substitutions, or combinations thereof.

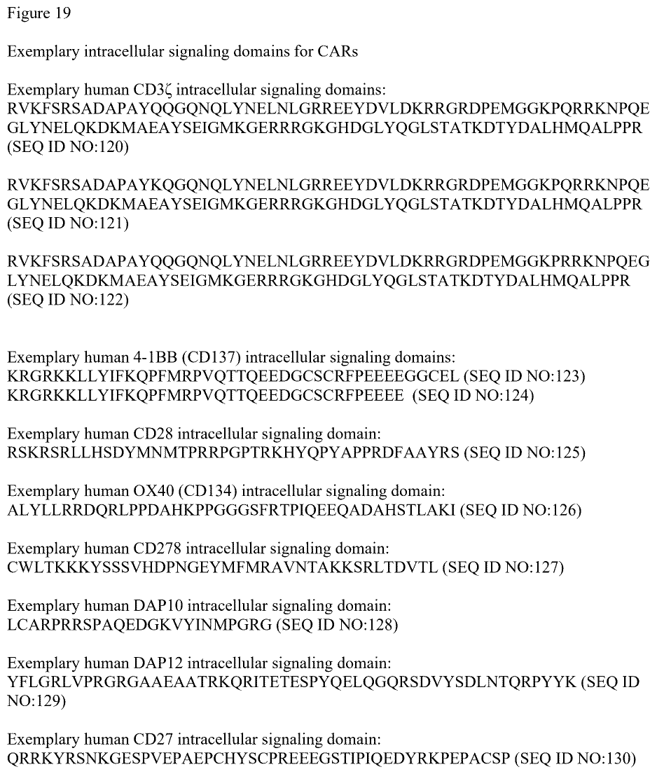

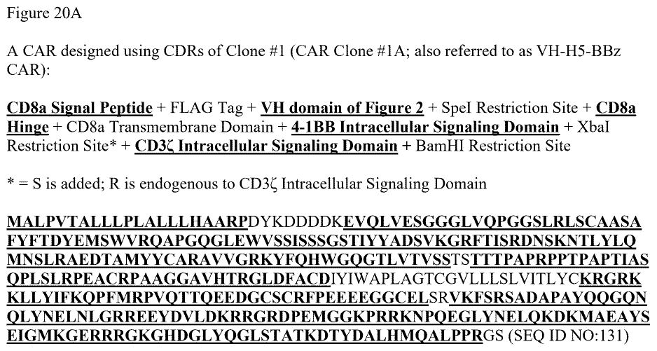

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 19. In some cases, a CAR provided herein can be designed to include at least one intracellular signaling domain that comprises, consists essentially of, or consists of one of the amino acid sequences set forth in FIG. 19 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 19 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

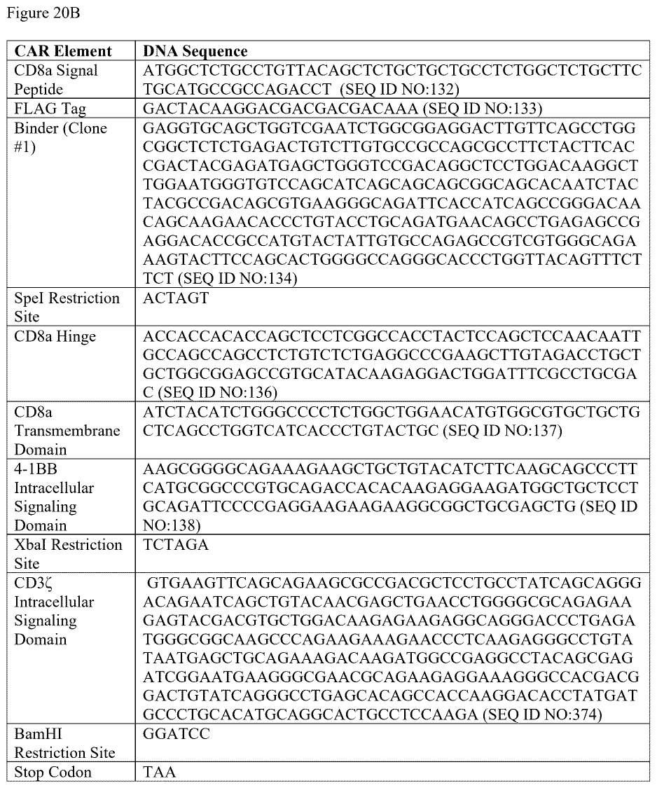

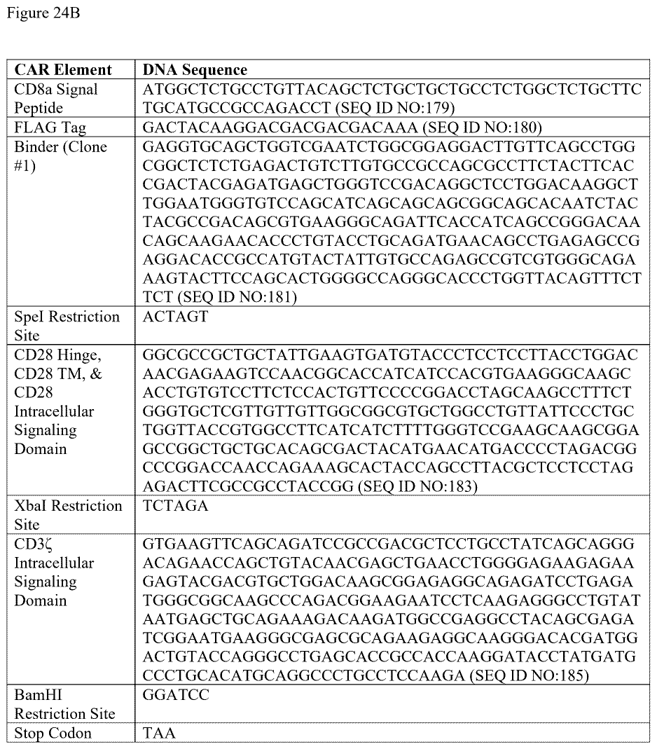

In some cases, a CAR targeting a CD135 polypeptide can be designed to include a VH domain comprising SEQ ID NO:1, SEQ ID NO:2, and SEQ ID NO:3, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:1, SEQ ID NO:2, and SEQ ID NO:3, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 20A-20B and 24A-24B).

In some cases, a CAR targeting a CD135 polypeptide can be designed to include a VH domain comprising SEQ ID NO:8, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:8, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 20A-20B and 24A-24B).

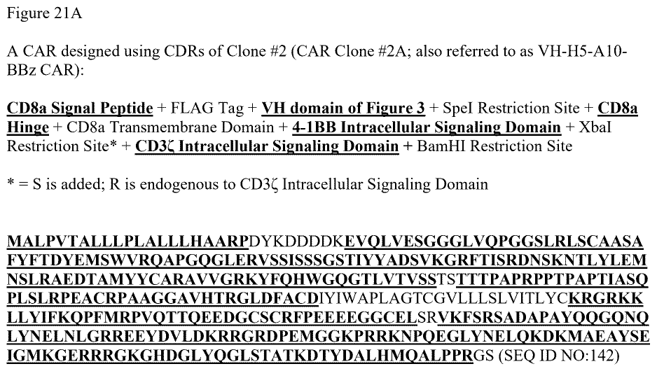

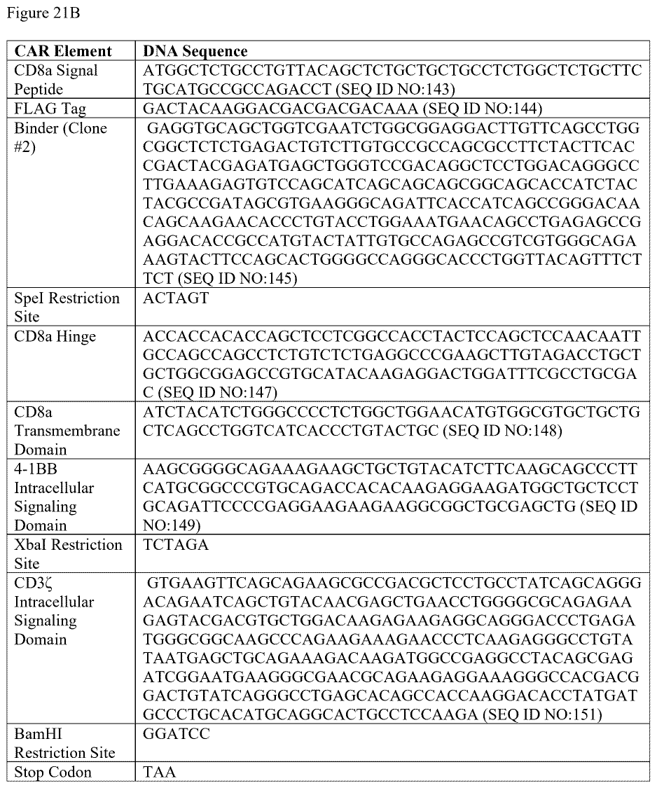

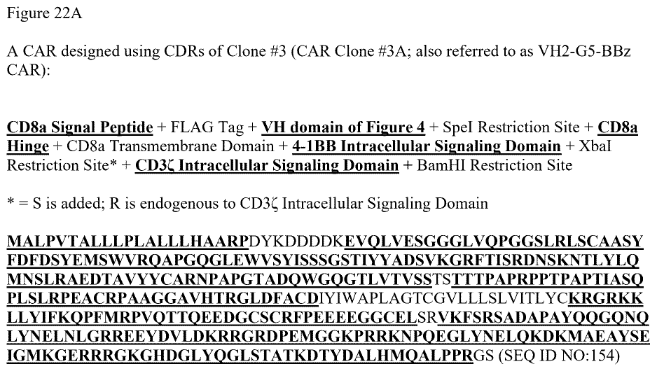

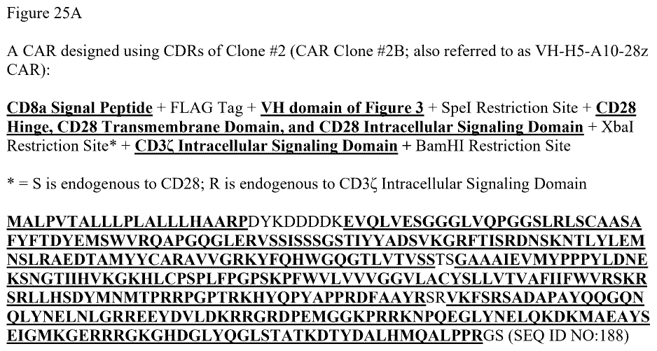

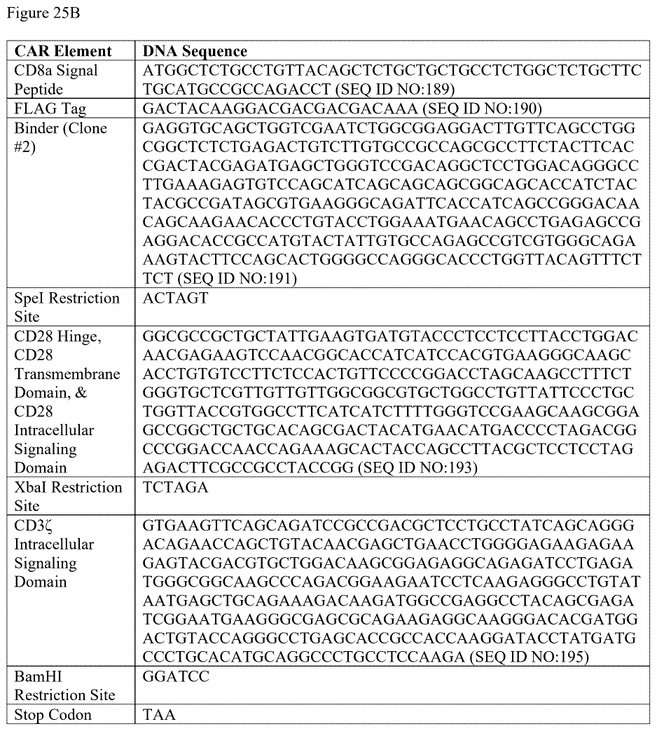

In some cases, a CAR targeting a CD135 polypeptide can be designed to include a VH domain comprising SEQ ID NO:9, SEQ ID NO:10, and SEQ ID NO:11, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:9, SEQ ID NO:10, and SEQ ID NO:11, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 21A-21B and 25A-25B).

In some cases, a CAR targeting a CD135 polypeptide can be designed to include a VH domain comprising SEQ ID NO:16, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:16, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 21A-21B and 25A-25B).

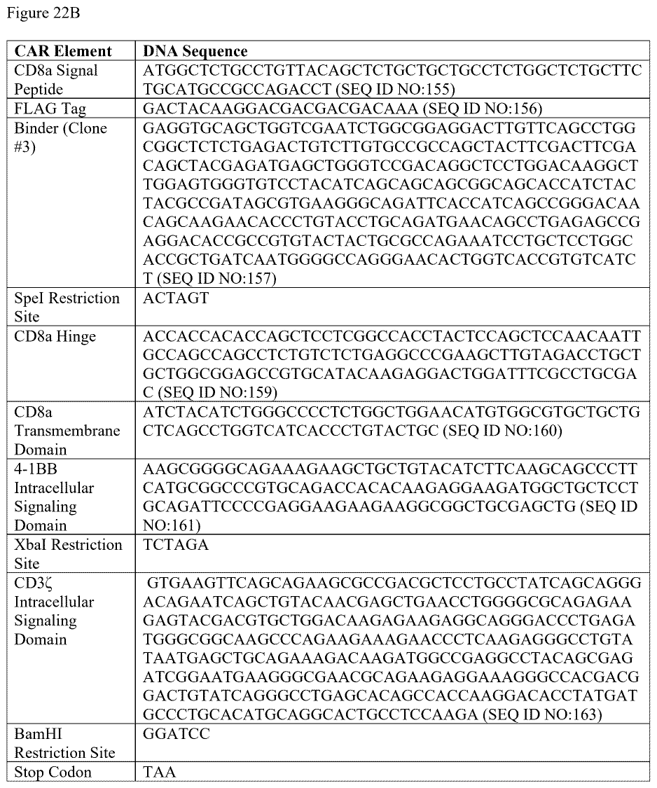

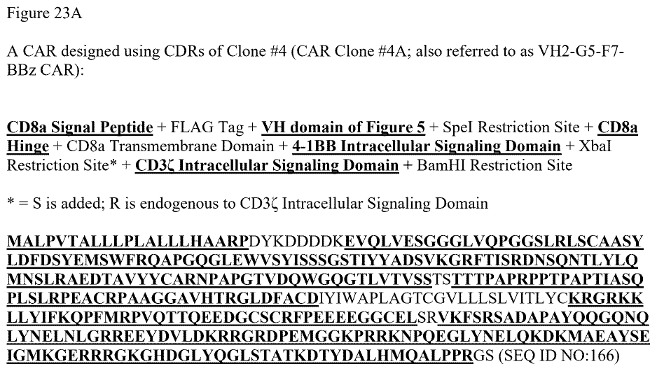

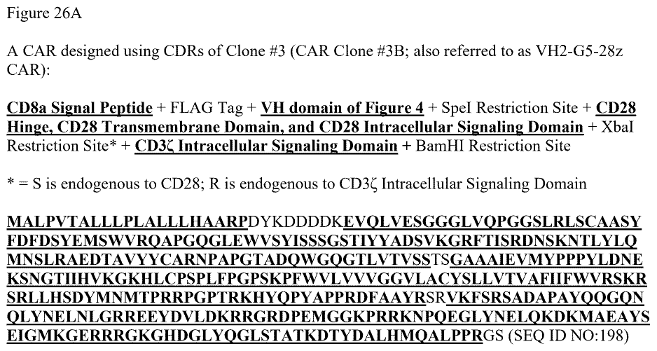

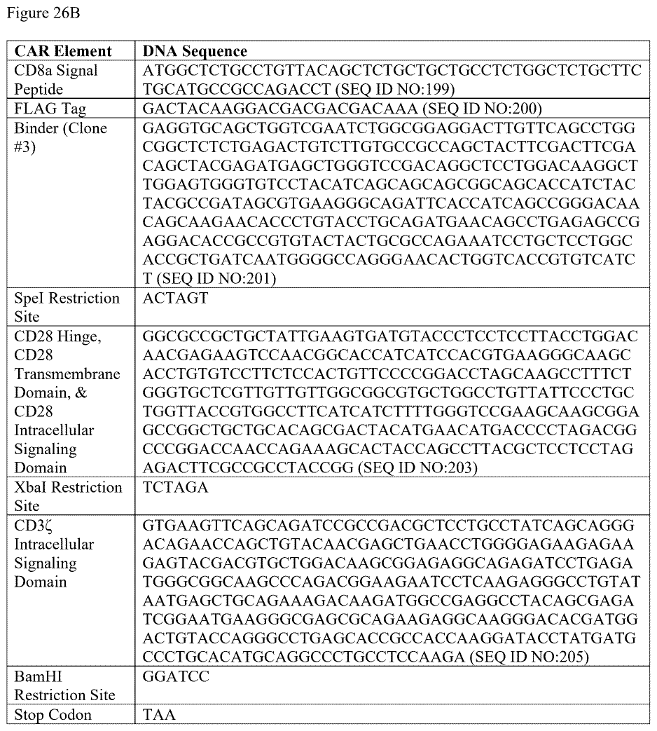

In some cases, a CAR targeting a CD135 polypeptide can be designed to include a VH domain comprising SEQ ID NO:17, SEQ ID NO:18, and SEQ ID NO:19, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:17, SEQ ID NO:18, and SEQ ID NO:19, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 22A-22B and 26A-26B).

In some cases, a CAR targeting a CD135 polypeptide can be designed to include a VH domain comprising SEQ ID NO:24, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:24, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 22A-22B and 26A-26B).

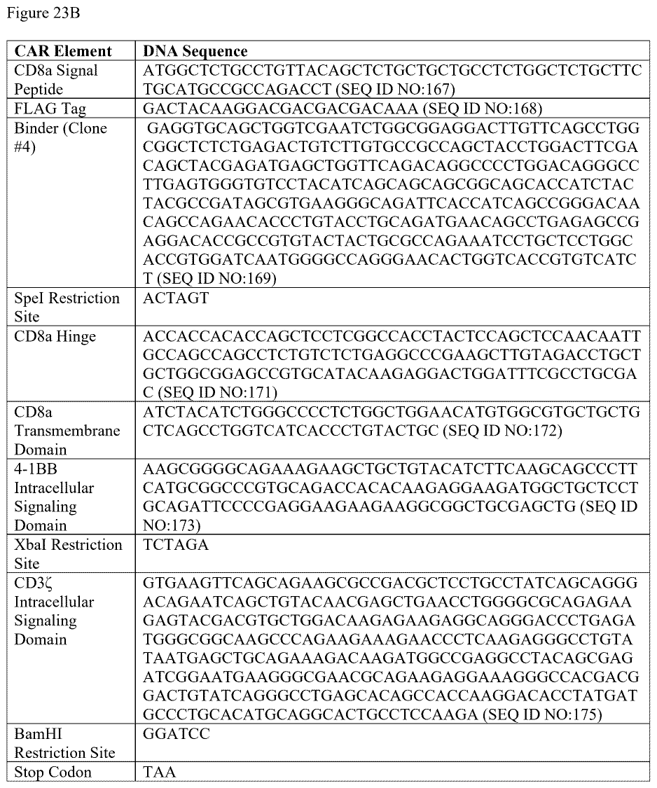

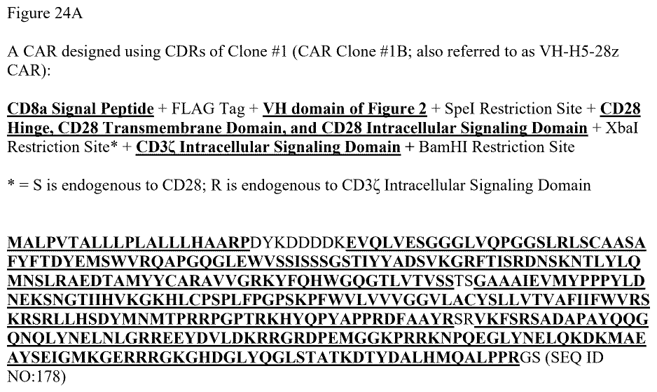

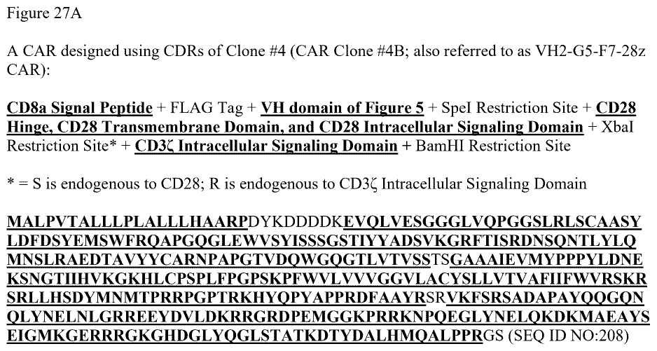

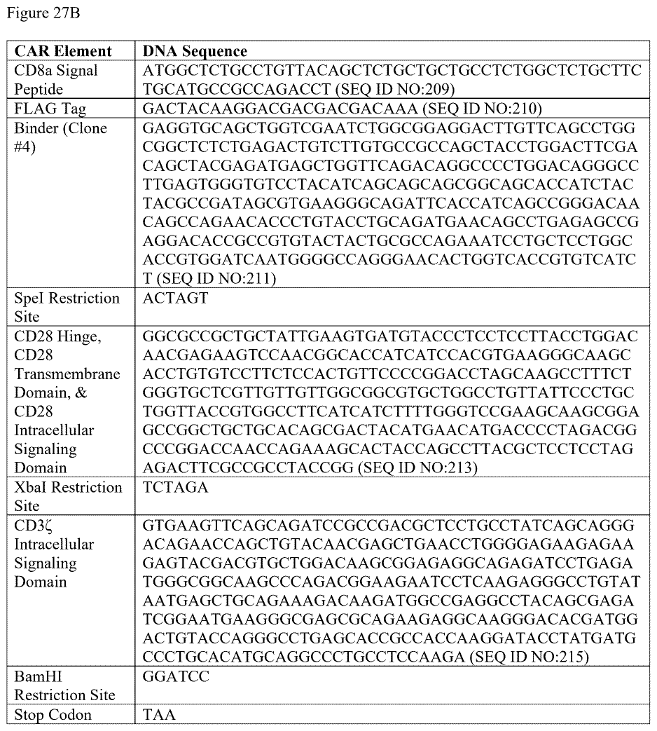

In some cases, a CAR targeting a CD135 polypeptide can be designed to include a VH domain comprising SEQ ID NO:25, SEQ ID NO:26, and SEQ ID NO:27, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:25, SEQ ID NO:26, and SEQ ID NO:27, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 23A-23B and 27A-27B).

In some cases, a CAR targeting a CD135 polypeptide can be designed to include a VH domain comprising SEQ ID NO:32, followed by a hinge such as a hinge/linker set forth in FIG. 13 or FIG. 17 (e.g., an IgG4-derived hinge, a CD8a hinge, or a linker plus IgG4-derived hinge), followed by a transmembrane domain such as a transmembrane domain set forth in FIG. 18 (e.g., a human CD28 transmembrane domain or a CD8a transmembrane domain), followed by one or more intracellular signaling domains such as one or more intracellular signaling domain set forth in FIG. 19 (e.g., a human 4-1BB intracellular signaling domain followed by a human CD3ζ intracellular signaling domain). For example, a CAR targeting a CD135 polypeptide can be designed to include a VH domain comprising SEQ ID NO:32, followed by SEQ ID NO:110 (or SEQ ID NO:249), followed by SEQ ID NO:115 (or SEQ ID NO:118), followed by SEQ ID NO:123 (or SEQ ID NO:125), followed by SEQ ID NO:122 (see, e.g., FIGS. 23A-23B and 27A-27B).

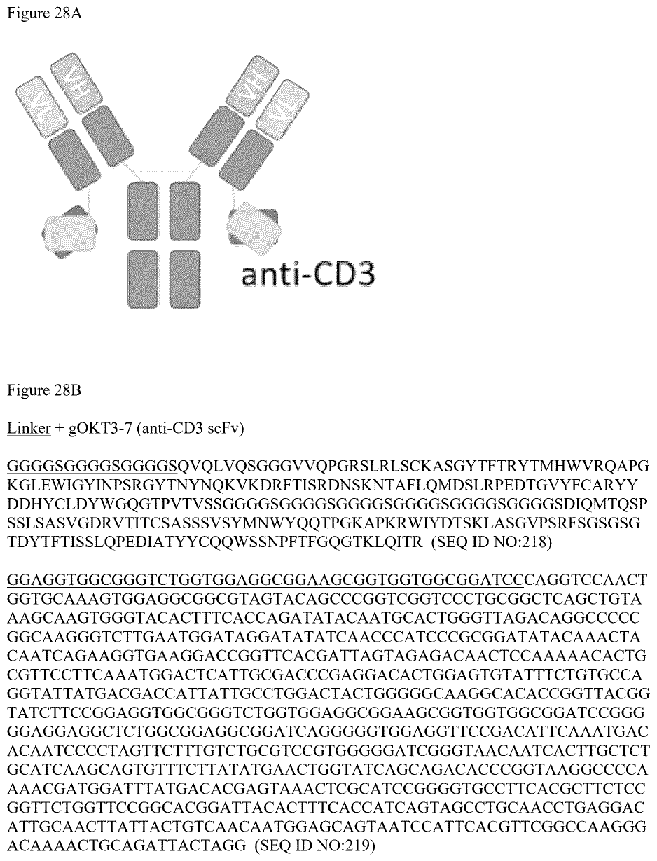

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a CD135 polypeptide (e.g., a human CD135 polypeptide) and at least one antigen binding domain having the ability to bind to an antigen expressed on the surface of a cell (e.g., a T cell or an NK cell). In some cases, a cell engager described herein can link a CD135⁺ cell (e.g., a CD135⁺ cancer cell) to another cell (e.g., a T cell or an NK cell) via the two or more antigen binding domains of the cell engager. An example of a cell engager structure of cell engagers provided herein includes, without limitation, the structure set forth in FIG. 28A. In some cases, the anti-CD3 scFv depicted in FIG. 28A can be replace with a different antigen binding domain having the ability to bind to an antigen expressed on the surface of a cell (e.g., a T cell or an NK cell).

When a cell engager includes an antigen binding domain having the ability to bind to a CD135 polypeptide (e.g., a human CD135 polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a CD135 polypeptide (e.g., a human CD135 polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a CD135 polypeptide (e.g., a human CD135 polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a CD135 polypeptide (e.g., a human CD135 polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment.

In some examples, a cell engager provided herein can be designed to include an antigen binding domain that includes a single set of three CDRs (e.g., a CDR1, CDR2, and CDR3) of an antibody domain (e.g., a VH domain) provided herein (e.g., SEQ ID NOs:1-3; SEQ ID NOs:9-11; SEQ ID NOs:17-19; or SEQ ID NOs:25-27).

In some cases, an antigen binding domain of a cell engager targeting a CD135 polypeptide can be designed to include a VH domain described herein or a scFv/Fab antibody described herein. In some cases, an antigen binding domain of a CAR described herein that has the ability to bind to a CD135 polypeptide (e.g., a human CD135 polypeptide) can be used as an antigen binding domain of a cell engager that targets CD135⁺ cells.

As described herein, a cell engager can be designed to include at least one antigen binding domain having the ability to bind to a CD135 polypeptide (e.g., a human CD135 polypeptide) and at least one other antigen binding domain. That at least one other antigen binding domain can have the ability to bind to any appropriate antigen expressed on the surface of a cell. For example, when designing a cell engager such as a BiTE to link a CD135⁺ cell and a T cell, the cell engager can include an antigen binding domain having the ability to bind to a CD135 polypeptide (e.g., a human CD135 polypeptide) and an antigen binding domain having the ability to bind to a polypeptide expressed on the surface of a T cell. Examples of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

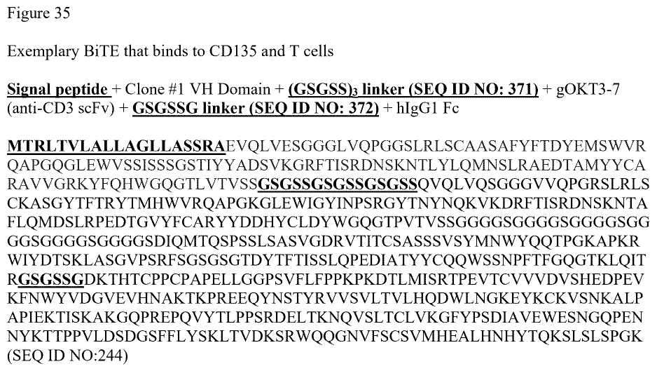

Examples of BiTE structures that can be used to make a BiTE provided herein include, without limitation, those set forth in FIGS. 35-38.

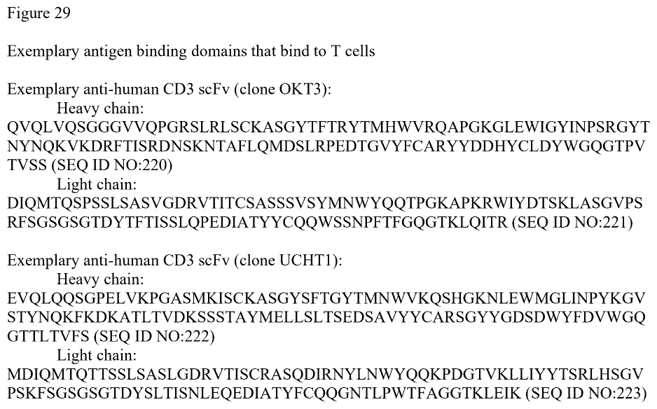

In some cases, a cell engager provided herein can be designed to include an antigen binding domain that comprises, consists essentially of, or consists of one of the amino acid sequences set forth in FIG. 29. In some cases, a cell engager provided herein can be designed to include an antigen binding domain that comprises, consists essentially of, or consists of one of the amino acid sequences set forth in FIG. 29 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 29 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a CD135⁺ cell and an NK cell, the cell engager can include an antigen binding domain having the ability to bind to a CD135 polypeptide (e.g., a human CD135 polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

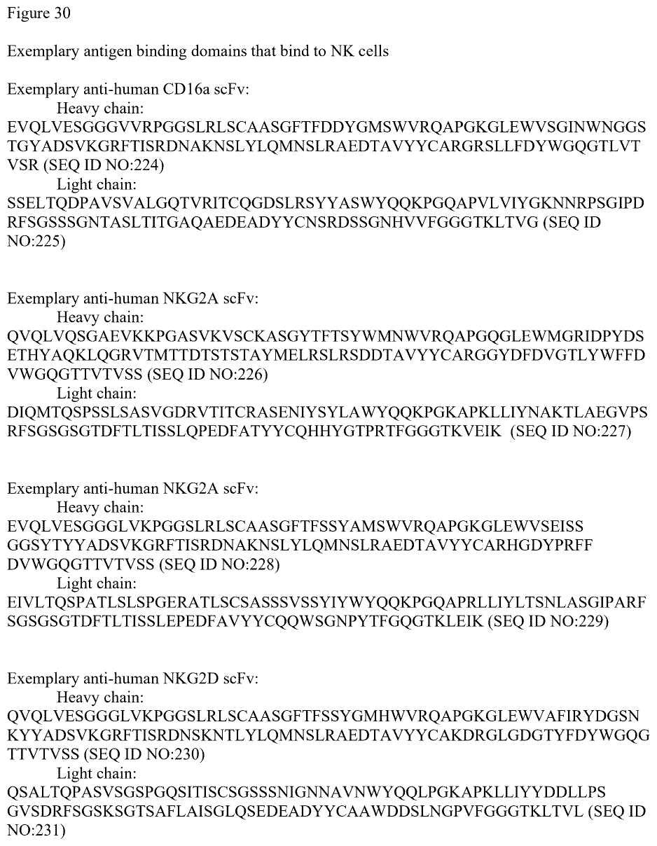

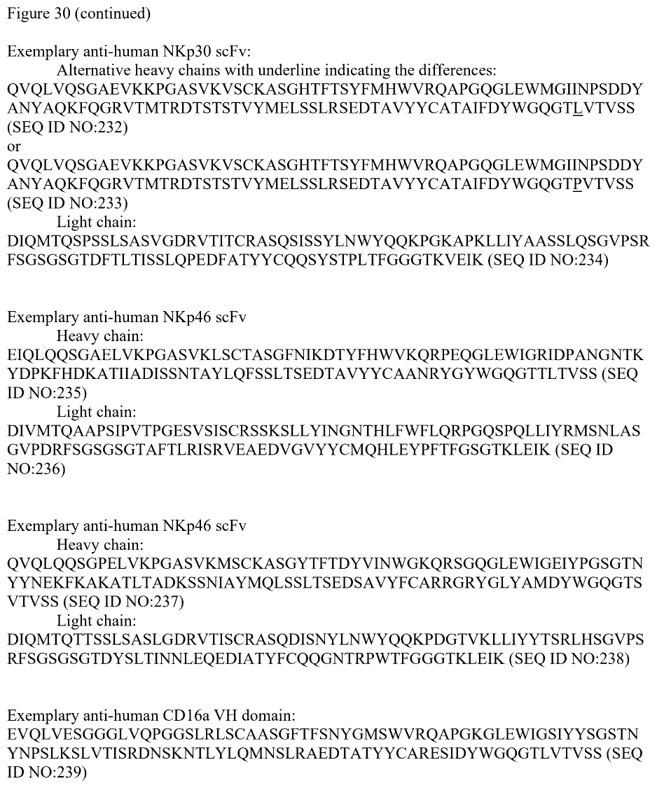

In some cases, a cell engager provided herein can be designed to include an antigen binding domain (e.g., a scFv or VH) that comprises, consists essentially of, or consists of one or more of the amino acid sequences set forth in FIG. 30. In some cases, a cell engager provided herein can be designed to include an antigen binding domain (e.g., a scFv or VH) that comprises, consists essentially of, or consists of one of the amino acid sequences set forth in FIG. 30 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 30 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 13. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:71) linker. In some cases, a hinge of a CAR described herein can be used as a linker to make a cell engager described herein. For example, any one of the sequences set forth in FIG. 17 can be used as a linker of a cell engager described herein.

In some cases, a cell engager provided herein can be designed to include a linker that comprises, consists essentially of, or consists of one of the amino acid sequences set forth in FIG. 13 or FIG. 17. In some cases, a cell engager provided herein can be designed to include a linker that comprises, consists essentially of, or consists of one of the amino acid sequences set forth in FIG. 13 or FIG. 17 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 13 or FIG. 17 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a CD135 polypeptide can be designed to include a VH domain comprising SEQ ID NO:1, SEQ ID NO:2, and SEQ ID NO:3, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 35).

In some cases, a cell engager (e.g., a BiTE) targeting a CD135 polypeptide can be designed to include a VH domain comprising SEQ ID NO:8, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 35).

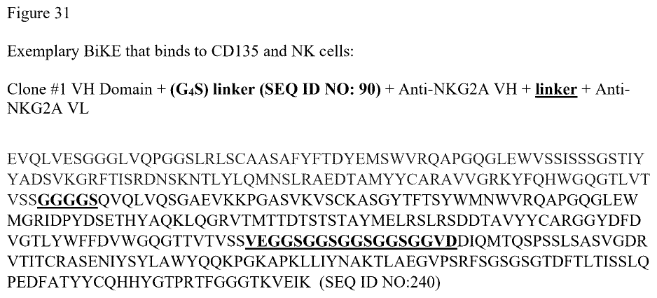

In some cases, a cell engager (e.g., a BiKE or a TriKE) targeting a CD135 polypeptide can be designed to include a VH domain comprising SEQ ID NO:1, SEQ ID NO:2, and SEQ ID NO:3, followed by a linker such as a linker/hinge set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 31).

In some cases, a cell engager (e.g., a BiKE or a TriKE) targeting a CD135 polypeptide can be designed to include a VH domain comprising SEQ ID NO:8, followed by a linker such as a hinge/linker set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 31).

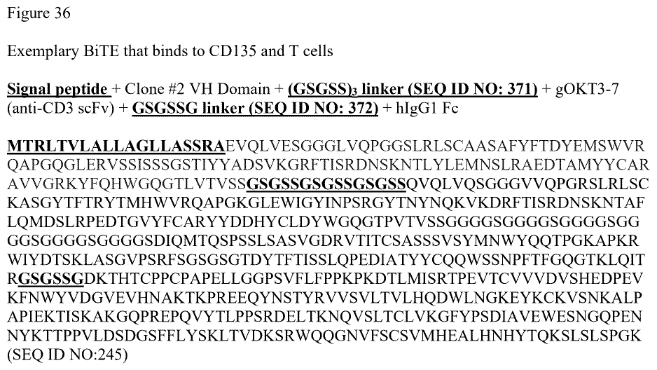

In some cases, a cell engager (e.g., a BiTE) targeting a CD135 polypeptide can be designed to include a VH domain comprising SEQ ID NO:9, SEQ ID NO:10, and SEQ ID NO:11, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 36).

In some cases, a cell engager (e.g., a BiTE) targeting a CD135 polypeptide can be designed to include a VH domain comprising SEQ ID NO:16, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 36).

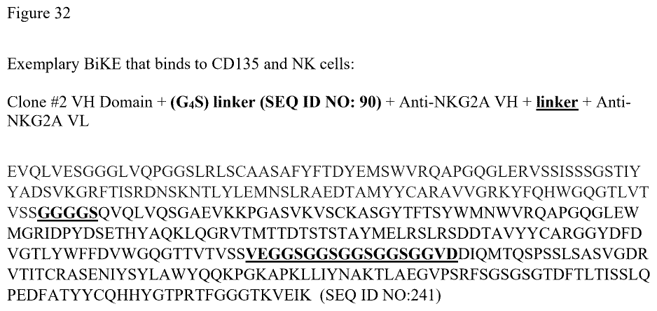

In some cases, a cell engager (e.g., a BiKE or a TriKE) targeting a CD135 polypeptide can be designed to include a VH domain comprising SEQ ID NO:9, SEQ ID NO:10, and SEQ ID NO:11, followed by a linker such as a linker/hinge set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 32).

In some cases, a cell engager (e.g., a BiKE or a TriKE) targeting a CD135 polypeptide can be designed to include a VH domain comprising SEQ ID NO:16, followed by a linker such as a hinge/linker set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 32).

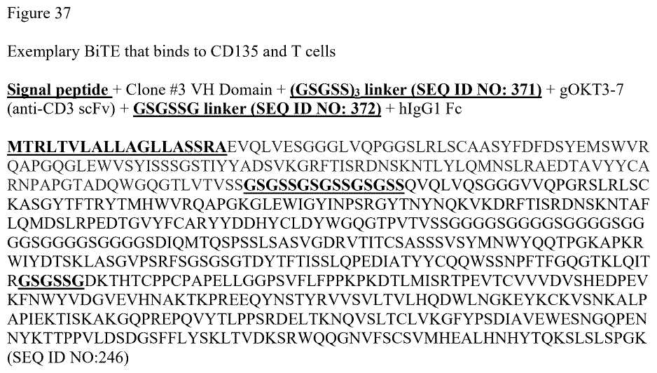

In some cases, a cell engager (e.g., a BiTE) targeting a CD135 polypeptide can be designed to include a VH domain comprising SEQ ID NO:17, SEQ ID NO:18, and SEQ ID NO:19, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 37).

In some cases, a cell engager (e.g., a BiTE) targeting a CD135 polypeptide can be designed to include a VH domain comprising SEQ ID NO:24, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 37).

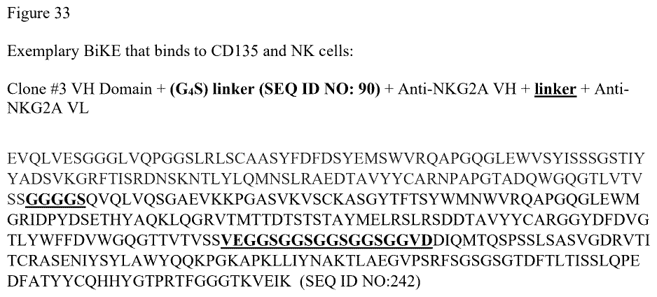

In some cases, a cell engager (e.g., a BiKE or a TriKE) targeting a CD135 polypeptide can be designed to include a VH domain comprising SEQ ID NO:17, SEQ ID NO:18, and SEQ ID NO:19, followed by a linker such as a linker/hinge set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 33).

In some cases, a cell engager (e.g., a BiKE or a TriKE) targeting a CD135 polypeptide can be designed to include a VH domain comprising SEQ ID NO:24, followed by a linker such as a hinge/linker set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 33).

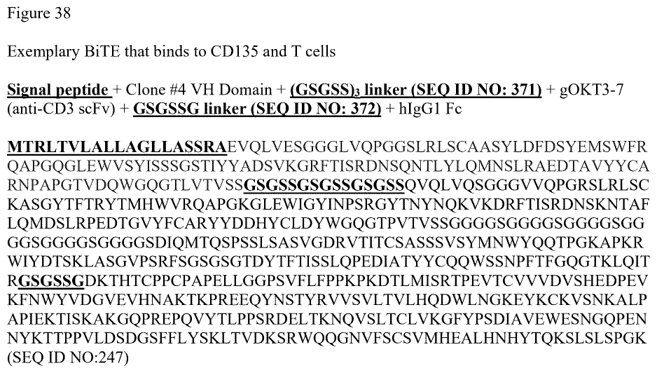

In some cases, a cell engager (e.g., a BiTE) targeting a CD135 polypeptide can be designed to include a VH domain comprising SEQ ID NO:25, SEQ ID NO:26, and SEQ ID NO:27, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 38).

In some cases, a cell engager (e.g., a BiTE) targeting a CD135 polypeptide can be designed to include a VH domain comprising SEQ ID NO:32, followed by a linker such as a linker/hinge set forth in FIG. 13 or FIG. 17 (e.g., SEQ ID NO:71 or 91), followed by an antigen binding domain having the ability to bind to a polypeptide expressed on the surface of a T cell (e.g., an anti-human CD3 scFv) (see, e.g., FIG. 38).

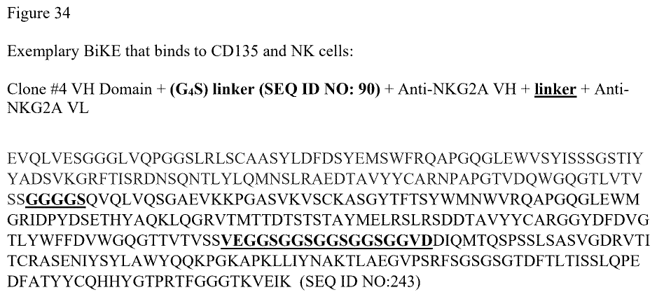

In some cases, a cell engager (e.g., a BiKE or a TriKE) targeting a CD135 polypeptide can be designed to include a VH domain comprising SEQ ID NO:25, SEQ ID NO:26, and SEQ ID NO:27, followed by a linker such as a linker/hinge set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 34).

In some cases, a cell engager (e.g., a BiKE or a TriKE) targeting a CD135 polypeptide can be designed to include a VH domain comprising SEQ ID NO:32, followed by a linker such as a hinge/linker set forth in FIG. 13 or FIG. 17 (e.g., SEQ ID NO:71 or 91), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 34).

In some cases, a cell engager (e.g., a BiTE) targeting a CD135 polypeptide can be designed to include an IgG (e.g., IgG1) configuration having (a) a heavy chain comprising, consisting essentially of, or consisting of a heavy chain variable domain comprising a set of three CDRs of a VH domain provided herein (e.g., SEQ ID NOs:1-3; SEQ ID NOs:9-11; SEQ ID NOs:17-19; or SEQ ID NOs:25-27), an Ig hinge, and constant domains (e.g., CH1, CH2, and CH3 domains) and (b) a light chain comprising, consisting essentially of, or consisting of a light chain variable domain, a constant domain (e.g., a kappa or lambda constant domain), and an antigen binding domain having the ability to bind to a polypeptide expressed on the surface of a T cell (e.g., an anti-human CD3 scFv) (see, e.g., FIG. 28A).

In some cases, a cell engager (e.g., a BiTE) targeting a CD135 polypeptide can be designed to include an IgG (e.g., IgG1) configuration having (a) a heavy chain comprising, consisting essentially of, or consisting of a heavy chain variable domain, an Ig hinge, and constant domains (e.g., CH1, CH2, and CH3 domains) and (b) a light chain comprising, consisting essentially of, or consisting of a light chain variable domain comprising a set of three CDRs of a VH domain provided herein (e.g., SEQ ID NOs:1-3; SEQ ID NOs:9-11; SEQ ID NOs:17-19; or SEQ ID NOs:25-27), a constant domain (e.g., a kappa or lambda constant domain), and an antigen binding domain having the ability to bind to a polypeptide expressed on the surface of a T cell (e.g., an anti-human CD3 scFv) (see, e.g., FIG. 28A).

In some cases, a cell engager (e.g., a BiKE) targeting a CD135 polypeptide can be designed to include an IgG (e.g., IgG1) configuration having (a) a heavy chain comprising, consisting essentially of, or consisting of a heavy chain variable domain comprising a set of three CDRs of a VH domain provided herein (e.g., SEQ ID NOs:1-3; SEQ ID NOs:9-11; SEQ ID NOs:17-19; or SEQ ID NOs:25-27), an Ig hinge, and constant domains (e.g., CH1, CH2, and CH3 domains) and (b) a light chain comprising, consisting essentially of, or consisting of a light chain variable domain, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a CD135 polypeptide can be designed to include an IgG (e.g., IgG1) configuration having (a) a heavy chain comprising, consisting essentially of, or consisting of a heavy chain variable domain, an Ig hinge, and constant domains (e.g., CH1, CH2, and CH3 domains) and (b) a light chain comprising, consisting essentially of, or consisting of a light chain variable domain comprising a set of three CDRs of a VH domain provided herein (e.g., SEQ ID NOs:1-3; SEQ ID NOs:9-11; SEQ ID NOs:17-19; or SEQ ID NOs:25-27), a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In one embodiment, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications). An example of such a binder having these CDRs and the ability to bind to a CD135 polypeptide (e.g., a human CD135 polypeptide) includes, without limitation, the VH domain set forth in FIG. 2.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) and having a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 2 can be designed to include framework regions as set forth in FIG. 2 or can be designed to include one or more framework regions from another antibody or antibody fragment. For example, an antibody domain (e.g., a VH domain) can be designed to include the three CDRs set forth in FIG. 2 and the framework regions set forth in FIG. 2 except that framework region 1 having the amino acid set forth in SEQ ID NO:4 is replaced with a framework region 1 having the amino acid set forth in SEQ ID NO:12, a framework region 1 having the amino acid set forth in SEQ ID NO:20, or a framework region 1 having the amino acid set forth in SEQ ID NO:28. In another example, an Fab or scFv can be designed to include (a) the three CDRs set forth in FIG. 2, (b) the framework regions set forth in FIG. 2-5, 9A, 10A, 11A, or 12A, and (c) a light chain variable domain.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:8. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:8. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD135 polypeptide (e.g., a human CD135 polypeptide) and can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR1 that consists essentially of the amino acid sequence set forth in SEQ ID NO:1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| EYEMS | 250 |
| EYDMS | 251 |
| DYDMS | 252 |
| EYEMH | 253 |
| DYDMH | 254 |
| DYEMH | 255 |
| EYEMT | 256 |
| DYDMT | 257 |
| DYEMT | 258 |
| EYDMH | 259 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| TISSSGSTIYYADSVKG | 260 |
| SITSSGSTIYYADSVKG | 261 |
| SISTSGSTIYYADSVKG | 262 |
| SISSTGSTIYYADSVKG | 263 |
| SISSSGSTIYYAESVKG | 264 |
| SISSSGSTIYYADTVKG | 265 |
| SISSSGSTIYYADSVRG | 266 |
| SISSSGSTIYYADSVRA | 267 |
| SISSSGSTIYYADSVKA | 268 |
| SLSSSGSTIYYADSVKG | 269 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| AVVGKKYFQH | 270 |
| AVVGRKYFNH | 271 |
| AVVGKKYFNH | 272 |
| AVVARKYFQH | 273 |
| GVVGRKYFQH | 274 |
| AVAGRKYFQH | 275 |
| AAVGRKYFQH | 276 |
| AVVGKKYFNH | 277 |
| GVVGKKYFQH | 278 |
| ALVGRRYFQH | 279 |

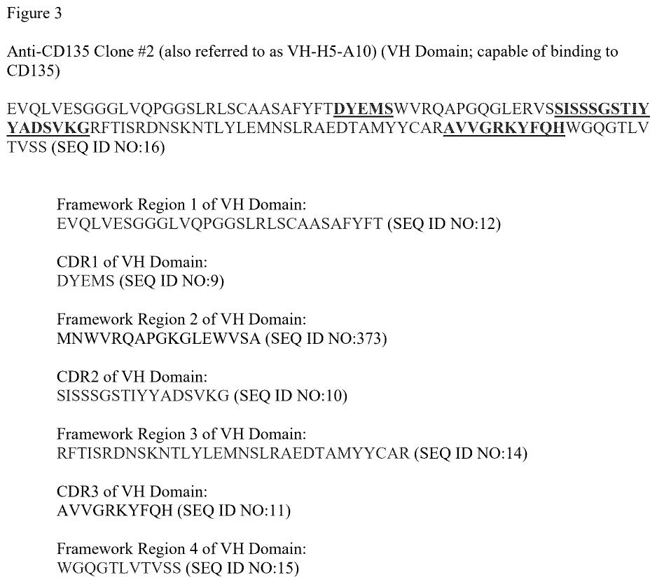

In one embodiment, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth in SEQ ID NO:11 (or a variant of SEQ ID NO:11 with one or two amino acid modifications). An example of such a binder having these CDRs and the ability to bind to a CD135 polypeptide (e.g., a human CD135 polypeptide) includes, without limitation, the VH domain set forth in FIG. 3.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) and having a heavy chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth in SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 3 can be designed to include framework regions as set forth in FIG. 3 or can be designed to include one or more framework regions from another antibody or antibody fragment. For example, an antibody domain (e.g., a VH domain) can be designed to include the three CDRs set forth in FIG. 3 and the framework regions set forth in FIG. 3 except that framework region 1 having the amino acid set forth in SEQ ID NO:12 is replaced with a framework region 1 having the amino acid set forth in SEQ ID NO:4, a framework region 1 having the amino acid set forth in SEQ ID NO:20, or a framework region 1 having the amino acid set forth in SEQ ID NO:28. In another example, an Fab or scFv can be designed to include (a) the three CDRs set forth in FIG. 3, (b) the framework regions set forth in FIG. 2-5, 9A, 10A, 11A, or 12A, and (c) a light chain variable domain.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:16, provided that the heavy chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:16, provided that the heavy chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD135 polypeptide (e.g., a human CD135 polypeptide) and can include a heavy chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| EYEMS | 280 |
| EYDMS | 281 |
| DYDMS | 282 |
| EYEMH | 283 |
| DYDMH | 284 |
| DYEMH | 285 |
| EYEMT | 286 |
| DYDMT | 287 |
| DYEMT | 288 |
| EYDMH | 289 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

| Sequence | SEQ ID NO: |
|---|---|
| TISSSGSTIYYADSVKG | 290 |
| SITSSGSTIYYADSVKG | 291 |
| SISTSGSTIYYADSVKG | 292 |
| SISSTGSTIYYADSVKG | 293 |
| SISSSGSTIYYAESVKG | 294 |
| SISSSGSTIYYADTVKG | 295 |
| SISSSGSTIYYADSVRG | 296 |
| SISSSGSTIYYADSVRA | 297 |
| SISSSGSTIYYADSVKA | 298 |
| SLSSSGSTIYYADSVKG | 299 |

As used herein, a “CDR3 that consists essentially of the amino acid sequence set forth in SEQ ID NO:11” is a CDR3 that has zero, one, or two amino acid substitutions within SEQ ID NO: 11, that has zero, one, two, three, four, or five amino acid residues directly preceding SEQ ID NO:11, and/or that has zero, one, two, three, four, or five amino acid residues directly following SEQ ID NO: 11, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| AVVGKKYFQH | 300 |
| AVVGRKYFNH | 301 |
| AVVGKKYFNH | 302 |
| AVVARKYFQH | 303 |
| GVVGRKYFQH | 304 |
| AVAGRKYFQH | 305 |
| AAVGRKYFQH | 306 |
| AVVGKKYFNH | 307 |
| GVVGKKYFQH | 308 |
| ALVGRRYFQH | 309 |

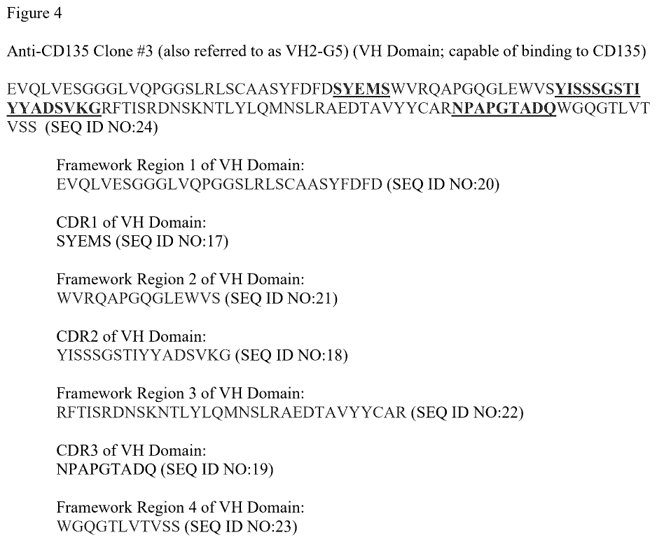

In one embodiment, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications). An example of such a binder having these CDRs and the ability to bind to a CD135 polypeptide (e.g., a human CD135 polypeptide) includes, without limitation, the VH domain set forth in FIG. 4.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) and having a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 4 can be designed to include framework regions as set forth in FIG. 4 or can be designed to include one or more framework regions from another antibody or antibody fragment. For example, an antibody domain (e.g., a VH domain) can be designed to include the three CDRs set forth in FIG. 4 and the framework regions set forth in FIG. 4 except that framework region 1 having the amino acid set forth in SEQ ID NO:20 is replaced with a framework region 1 having the amino acid set forth in SEQ ID NO:4, a framework region 1 having the amino acid set forth in SEQ ID NO:12, or a framework region 1 having the amino acid set forth in SEQ ID NO:28. In another example, an Fab or scFv can be designed to include (a) the three CDRs set forth in FIG. 4, (b) the framework regions set forth in FIG. 2-5, 9A, 10A, 11A, or 12A, and (c) a light chain variable domain.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:24. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:24. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD135 polypeptide (e.g., a human CD135 polypeptide) and can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR1 that consists essentially of the amino acid sequence set forth in SEQ ID NO:17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| TYEMS | 310 |
| SYEMT | 311 |
| SYDMS | 312 |
| SYEMH | 313 |
| SYEMA | 314 |
| SYEMN | 315 |
| TYEMH | 316 |
| TYEMN | 317 |
| SYDMA | 318 |
| SYDMH | 319 |

As used herein, a “CDR2 that consists essentially of the amino acid sequence set forth in SEQ ID NO: 18” is a CDR2 that has zero, one, or two amino acid substitutions within SEQ ID NO: 18, that has zero, one, two, three, four, or five amino acid residues directly preceding SEQ ID NO:18, and/or that has zero, one, two, three, four, or five amino acid residues directly following SEQ ID NO: 18, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR2 that consists essentially of the amino acid sequence set forth in SEQ ID NO: 18 include, without limitation, those set forth in Table 8.

TABLE 8
Exemplary CDR2s that consist essentially of the
amino acid sequence set forth in SEQ ID NO: 18.

| Sequence | SEQ ID NO: |
|---|---|
| YITSSGSTIYYADSVKG | 320 |
| YISTSGSTIYYADSVKG | 321 |
| YISSTGSTIYYADSVKG | 322 |
| YISSSGTTIYYADSVKG | 323 |
| YISSSGSSIYYADSVKG | 324 |
| YISSSGSTIYYAESVKG | 325 |
| YISSSGSTIYYADTVKG | 326 |
| YISSSGSTIYYADSVRG | 327 |
| YISSSGSTIYYADSVKA | 328 |
| YISSSGSTIYYADSAKG | 329 |

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR3 that consists essentially of the amino acid sequence set forth in SEQ ID NO:19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| NPAPGTADN | 330 |
| NPAPGTAEQ | 331 |
| NPAPGSADQ | 332 |
| QPAPGTADQ | 333 |
| NPAPGTADH | 334 |
| QPAPGSADQ | 335 |
| QPAPGTADN | 336 |
| QPAPGSADH | 337 |
| NPAPGTAEH | 338 |
| QPAPGSAEN | 339 |

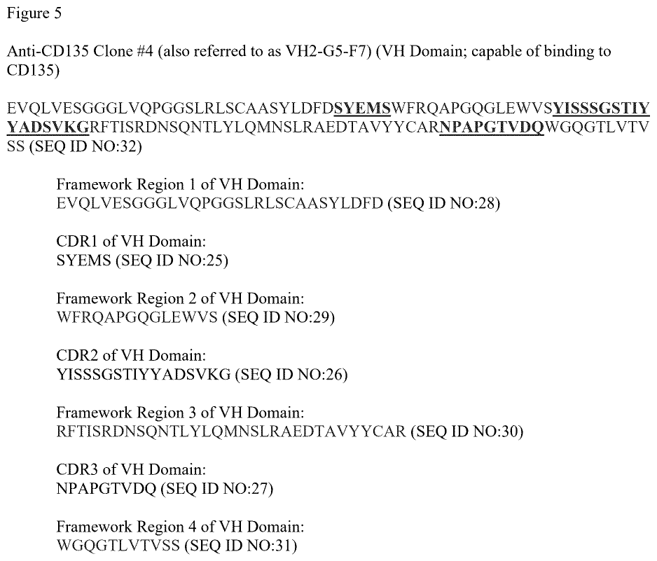

In one embodiment, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth in SEQ ID NO:27 (or a variant of SEQ ID NO:27 with one or two amino acid modifications). An example of such a binder having these CDRs and the ability to bind to a CD135 polypeptide (e.g., a human CD135 polypeptide) includes, without limitation, the VH domain set forth in FIG. 5.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) and having a heavy chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth in SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 5 can be designed to include framework regions as set forth in FIG. 5 or can be designed to include one or more framework regions from another antibody or antibody fragment. For example, an antibody domain (e.g., a VH domain) can be designed to include the three CDRs set forth in FIG. 5 and the framework regions set forth in FIG. 5 except that framework region 1 having the amino acid set forth in SEQ ID NO:28 is replaced with a framework region 1 having the amino acid set forth in SEQ ID NO:4, a framework region 1 having the amino acid set forth in SEQ ID NO:12, or a framework region 1 having the amino acid set forth in SEQ ID NO:20. In another example, an Fab or scFv can be designed to include (a) the three CDRs set forth in FIG. 5, (b) the framework regions set forth in FIG. 2-5, 9A, 10A, 11A, or 12A, and (c) a light chain variable domain.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain that includes an amino acid sequence having at least 90 percent identity to the amino acid sequence set forth in SEQ ID NO:32, provided that the heavy chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:32, provided that the heavy chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain having the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD135 polypeptide (e.g., a human CD135 polypeptide) and can include a heavy chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD135 polypeptide (e.g., a human CD135 polypeptide) can include a heavy chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR1 that consists essentially of the amino acid sequence set forth in SEQ ID NO:25 include, without limitation, those set forth in Table 10.

TABLE 10
Exemplary CDR1s that consist essentially of the
amino acid sequence set forth in SEQ ID NO: 25.

| Sequence | SEQ ID NO: |
|---|---|
| TYEMS | 340 |
| SYEMT | 341 |
| SYDMS | 342 |
| SYEMH | 343 |
| SYEMA | 344 |
| SYEMN | 345 |
| TYEMH | 346 |
| TYEMN | 347 |
| SYDMA | 348 |
| SYDMH | 349 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR2 that consists essentially of the amino acid sequence set forth in SEQ ID NO:26 include, without limitation, those set forth in Table 11.

TABLE 11
Exemplary CDR2s that consist essentially of the
amino acid sequence set forth in SEQ ID NO: 26.

| Sequence | SEQ ID NO: |
|---|---|
| YITSSGSTIYYADSVKG | 350 |
| YISTSGSTIYYADSVKG | 351 |
| YISSTGSTIYYADSVKG | 352 |
| YISSSGTTIYYADSVKG | 353 |
| YISSSGSSIYYADSVKG | 354 |
| YISSSGSTIYYAESVKG | 355 |
| YISSSGSTIYYADTVKG | 356 |
| YISSSGSTIYYADSVRG | 357 |
| YISSSGSTIYYADSVKA | 358 |
| YISSSGSTIYYADSAKG | 359 |

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a CD135 polypeptide (e.g., a human CD135 polypeptide). Examples of a CDR3 that consists essentially of the amino acid sequence set forth in SEQ ID NO:27 include, without limitation, those set forth in Table 12.

TABLE 12
Exemplary CDR3s that consist essentially of the
amino acid sequence set forth in SEQ ID NO: 27.

| Sequence | SEQ ID NO: |
|---|---|
| NPAPGTVDN | 360 |
| NPAPGTVEQ | 361 |
| NPAPGSVDQ | 362 |
| QPAPGTVDQ | 363 |
| NPAPGTVDH | 364 |
| QPAPGSVDQ | 365 |
| QPAPGTVDN | 366 |
| QPAPGSADH | 367 |
| NPAPGTAEH | 368 |
| QPAPGSAEN | 369 |

When designing a single chain antibody (e.g., a scFv) having a heavy chain variable domain and a light chain variable domain, the two regions can be directly connected or can be connected using any appropriate linker sequence. For example, a heavy chain variable domain can be connected to a light chain variable domain via a linker sequence. Examples of linker sequences that can be used to connect a heavy chain variable domain and a light chain variable domain to create a scFv include, without limitation, those linkers set forth in FIG. 13.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

The percent sequence identity between a particular amino acid or nucleic acid sequence and an amino acid or nucleic acid sequence referenced by a particular sequence identification number is determined as follows. First, an amino acid or nucleic acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO:8, SEQ ID NO:16, or SEQ ID NO:24), followed by multiplying the resulting value by 100. For example, an amino acid sequence that has 100 matches when aligned with the sequence set forth in SEQ ID NO:8 is 84 percent identical to the sequence set forth in SEQ ID NO:8 (i.e., 100÷119×100=84.0). It is noted that the percent sequence identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a CD135 polypeptide (e.g., a human CD135 polypeptide) are further described in Table 13.

TABLE 13
Representative number of binders.

| | SEQ ID NOs | SEQ ID NOs | SEQ ID NO |
|---|---|---|---|
| | of Heavy | of Heavy Chain | of Heavy |
| Clone # | Chain Variable | Variable Domain | Chain Variable |
| (Antibody type) | Domain CDRs | Framework Regions | Domain |
| #1 (VH domain) | 1, 2, 3 | 4, 5, 6, 7 | 8 |
| #2 (VH domain) | 9, 10, 11 | 12, 13, 14, 15 | 16 |
| #3 (VH domain) | 17, 18, 19 | 20, 21, 22, 23 | 24 |
| #4 (VH domain) | 25, 26, 27 | 28, 29, 30, 31 | 32 |

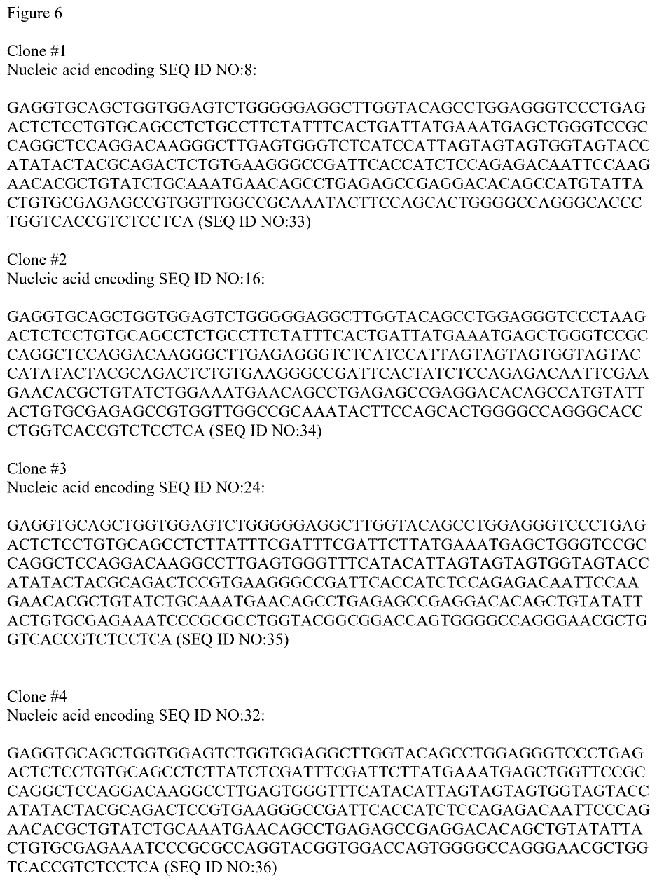

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 6 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a CD135 polypeptide (e.g., a human CD135 polypeptide). In some cases, a bispecific binder provided herein can be designed to bind to two different epitopes of the same CD135 polypeptide (e.g., a human CD135 polypeptide). In some cases, a bispecific binder provided herein can bind to a CD135 polypeptide (e.g., a human CD135 polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a CD135 polypeptide (e.g., a human CD135 polypeptide) and (b) at least one drug covalently linked directly or indirectly to that antigen binding domain. Any appropriate binder (e.g., an antibody, antigen binding fragment, and/or antibody domain) provided herein and having the ability to bind to a CD135 polypeptide (e.g., a human CD135 polypeptide) can be used as an antigen binding domain to make an ADC described herein. For example, any of the binders set forth in Table 13 can be used to make an ADC having the ability to bind to a CD135 polypeptide (e.g., a human CD135 polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a CD135 polypeptide (e.g., a human CD135 polypeptide) to form an ADC provided herein. For example, cleavable or non-cleavable ADC linkers can be used to covalently attach one or more drugs to an antigen binding domain having the ability to bind to a CD135 polypeptide (e.g., a human CD135 polypeptide) to form an ADC provided herein. Examples of ADC linkers can be used to covalently attach one or more drugs to an antigen binding domain having the ability to bind to a CD135 polypeptide (e.g., a human CD135 polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a VH domain such as a VH domain as set forth in any one of FIGS. 2-5. In another example, an isolated nucleic acid molecule provided herein can include a nucleic acid sequence encoding a CAR or cell engager (e.g., a BiTE, BiKE, or TriKE) described herein. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a CD135 polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a CD135 polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g., a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g., a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a CD135 polypeptide (e.g., a human CD135 polypeptide) can be formulated as a pharmaceutical composition for administration to a mammal (e.g., a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a CD135 polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a CD135 polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, leukemia such as AML and/or ALL, prostate carcinoma, and Ewing sarcoma. In some cases, a mammal (e.g., a human) having a CD135⁺ cancer (e.g., a CD135⁺ AML or a CD135⁺ ALL) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), intratumorally (e.g., via an intratumoral injection), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a CD135 polypeptide (e.g., a human CD135 polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a CD135 polypeptide (e.g., a human CD135 polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a CD135 polypeptide (e.g., a human CD135 polypeptide). In some cases, a binder (e.g., an antibody, antigen binding fragment, and/or antibody domain) provided herein can be immobilized, e.g., on a support, and retention of a CD135 polypeptide (e.g., a human CD135 polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a CD135 polypeptide (e.g., a human CD135 polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human CD135 Polypeptide

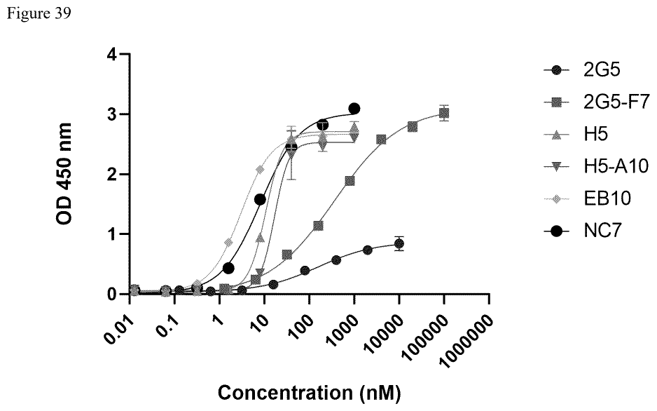

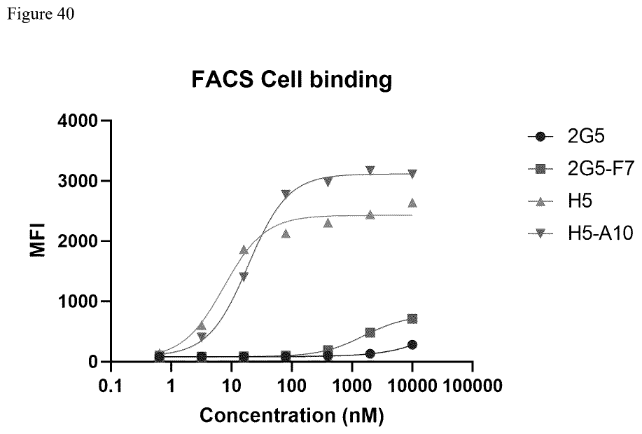

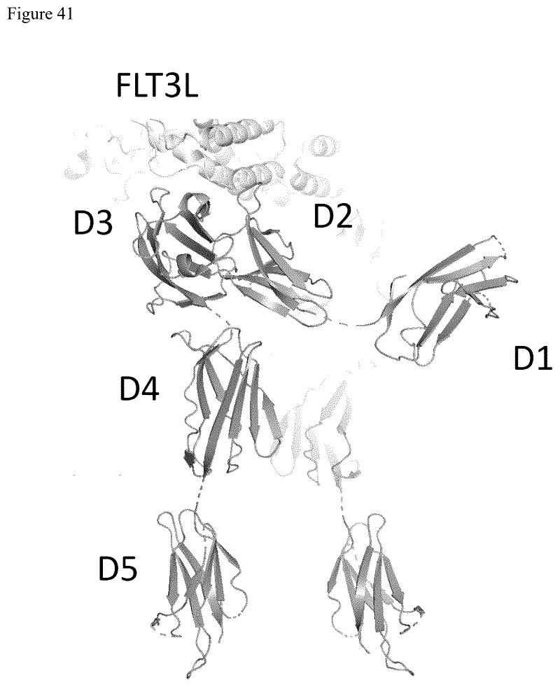

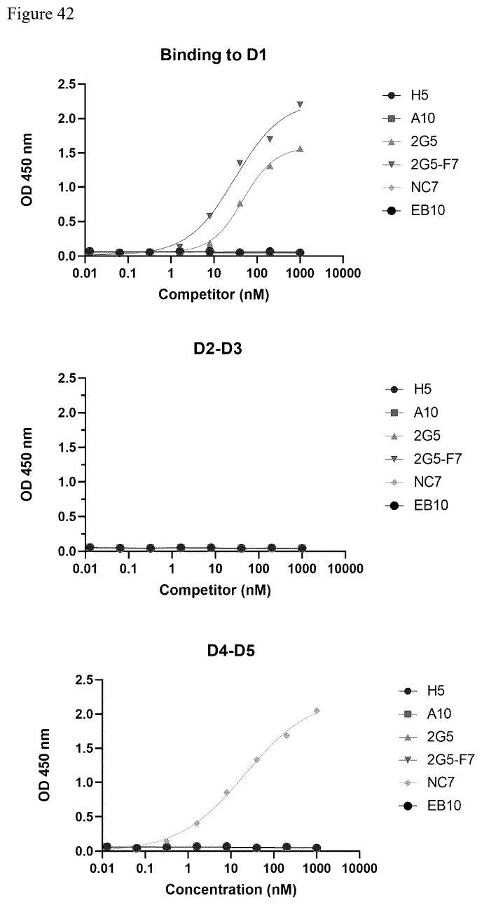

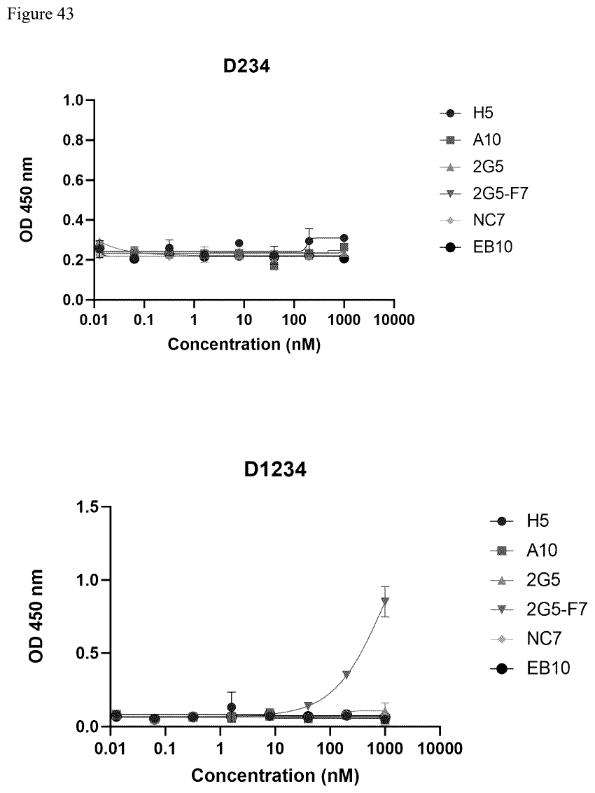

Large phage displayed antibody domain libraries were panned and screened to identify binders that bind to a human CD135 polypeptide using the amino acid sequence set forth in SEQ ID NO:39 (FIG. 1). To identify such binders, a polypeptide having amino acid residues 27 to 543 of human CD135 polypeptide set forth in FIG. 1 was biotin-labeled, and the biotinylated CD135 polypeptide was used for panning of human VH domain phage-displayed libraries. Two VH domains (Clones #1 and #3; FIGS. 2 and 4) were identified. Clones #2 and #4 were obtained by in vitro affinity maturation. Binding affinity and specificity to a human CD135 polypeptide were tested using an ELISA and flow cytometry (FIGS. 39 and 40). Clones #1-#4 exhibited a broad range of affinities, binding to a human CD135 polypeptide having EC₅₀ values of 2.7 nM, 2.5 nM, 157.6 nM, and 360.4 nM, respectively. All the clones bound to MV4-11 cells displaying human CD135, but did not bind to CHO-K1 cells lacking expression of human CD135, demonstrating that they can bind to CD135 polypeptides present on cells (FIG. 40). Clones #1 and #2 were determined to bind to a conformational epitope in proximity to domain 5 of a human CD135 polypeptide; and Clone #3 and 4 were determined to bind to domain 1 of a human CD135 polypeptide (FIGS. 42 and 43; see, also FIG. 41).

### Example 2—Designing CARs from Binders Having the Ability to Bind to a Human CD135 Polypeptide

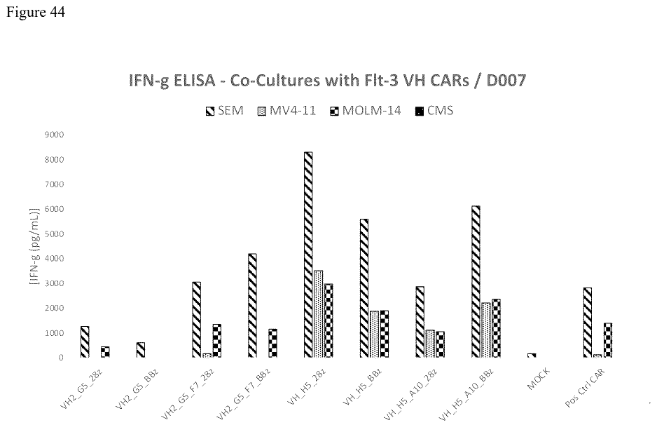

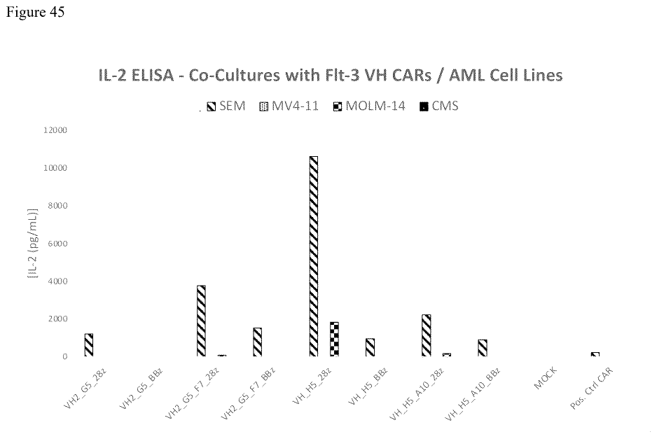

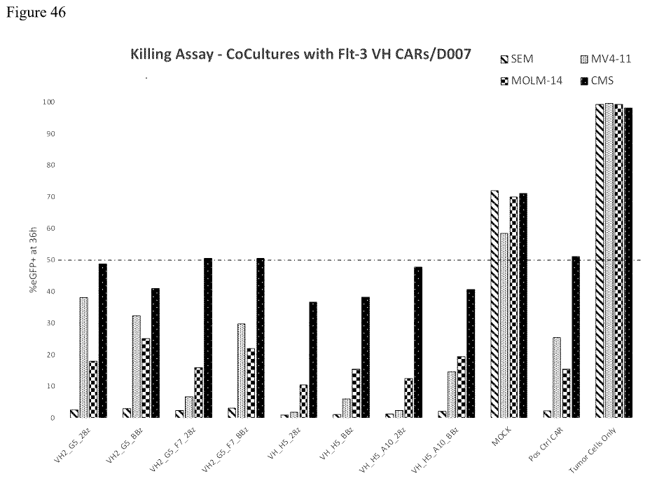

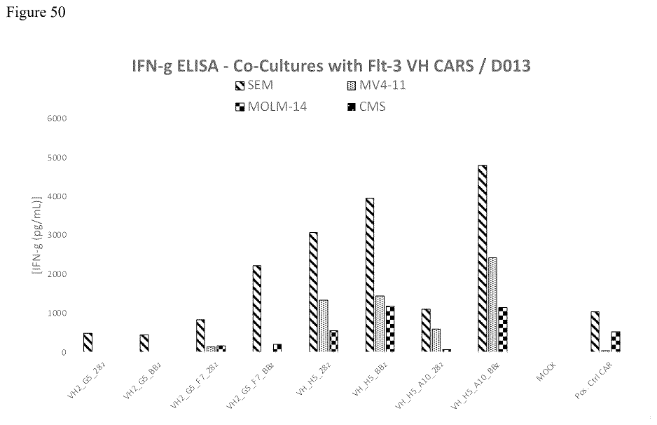

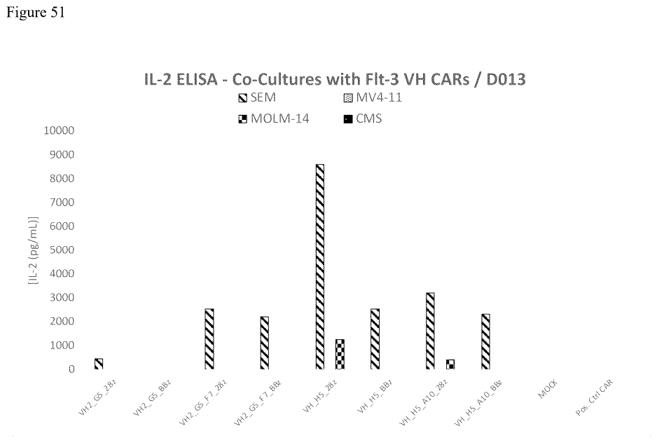

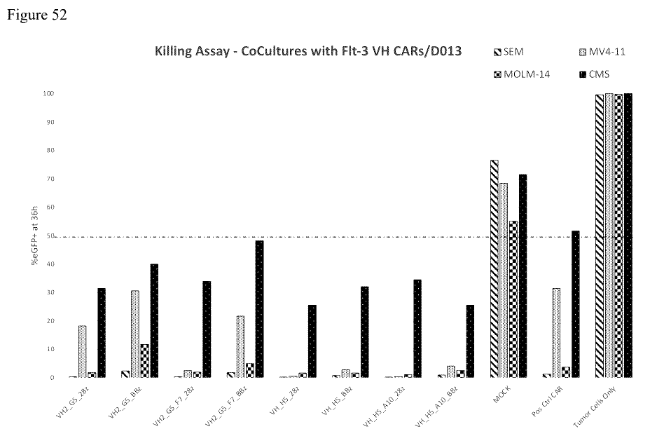

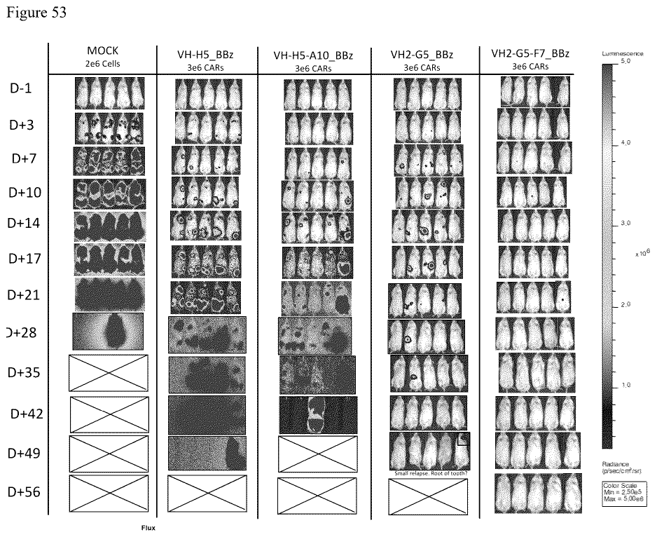

Clones #1-#4 were used to make CAR Clones #1A, #2A, #3A, #4A, #1B, #2B, #3B, and #4B as shown in FIGS. 20-27, respectively. The nucleic acid encoding these CARs under the control of a CMV promoter were introduced into human primary T cells. CAR-expressing T cells for CAR Clones #1A, #2A, #3A, #4A, #1B, #2B, #3B, and #4B were incubated for 16 hours with target cells (e.g., SEM, MV4-11, MOLM-14, or CMS cells) at a 1:1 ratio of T cells to target cells. Effector cells expressing CAR Clones #1A, #2A, #3A, #4A, #1B, #2B, #3B, and #4B exhibited cytokine release and killing of the CD135⁺ target cells and exhibited no or limited killing of CD135⁻ cells (e.g., CMS cells) (FIGS. 44-46 and 50-52).

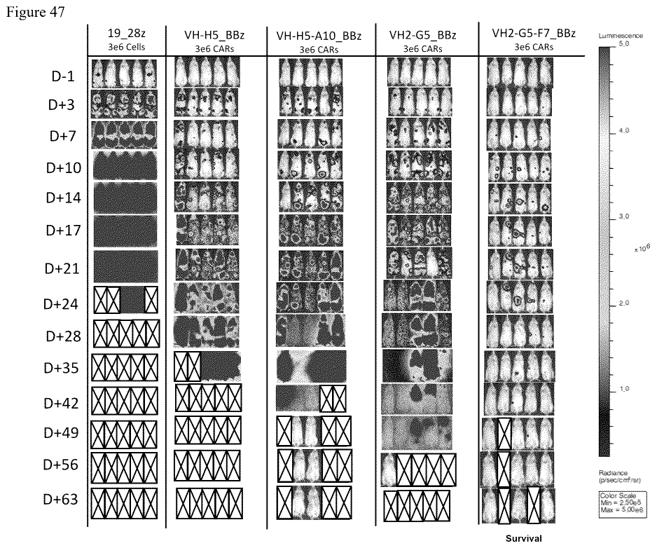

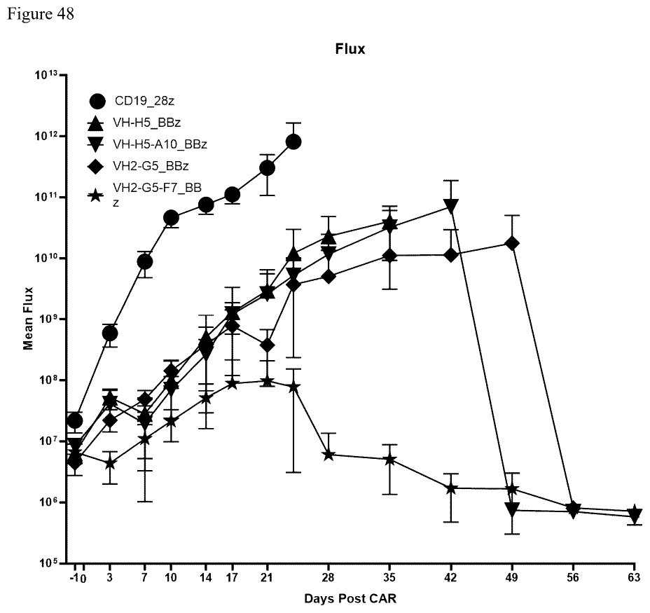

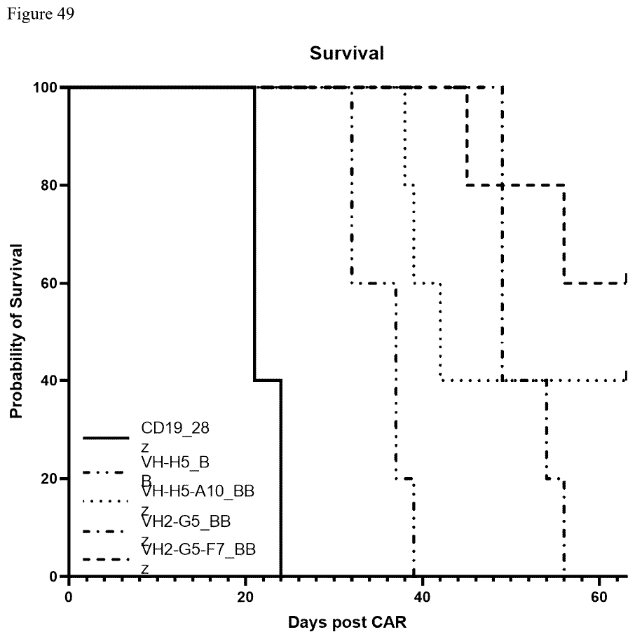

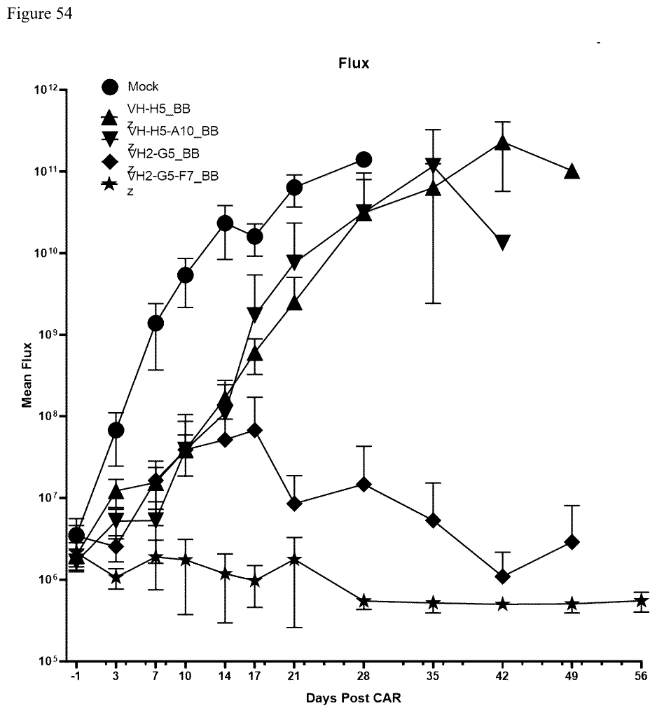

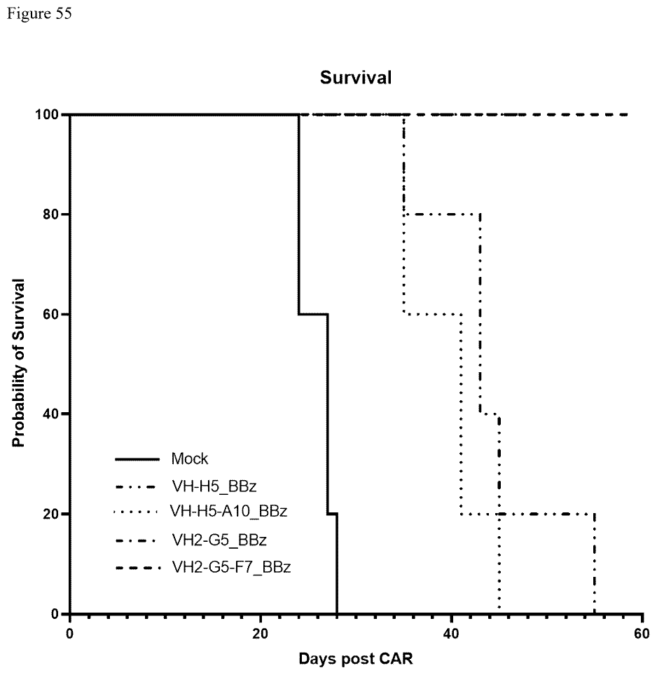

Mice administered 1×10⁶ MOLM-14 cancer cells were administered (a) 3×10⁶ T cells designed to express CAR Clone #1A, #2A, #3A, or #4A, (b) 3×10⁶ T cells designed to express a control CAR (e.g., CAR 19_28z), or (c) 2×10⁶ T cells not designed to express any CARs (mock cells). Mice administered T cells designed to express CAR Clone #1A, #2A, #3A, or #4A exhibited longer survival than mice administered T cells designed to express a control CAR and mice administered mock cells (FIGS. 47-49 and 53-55). Mice administered T cells designed to express CAR Clone #4A exhibited complete and sustained clearance of the MOLM-14 leukemia cancer (FIGS. 47-49 and 53-55).

These results demonstrate that CARs can be designed and used to create CAR T cells having the ability to kill cells expressing human CD135 such as CD135⁺ cancer cells.

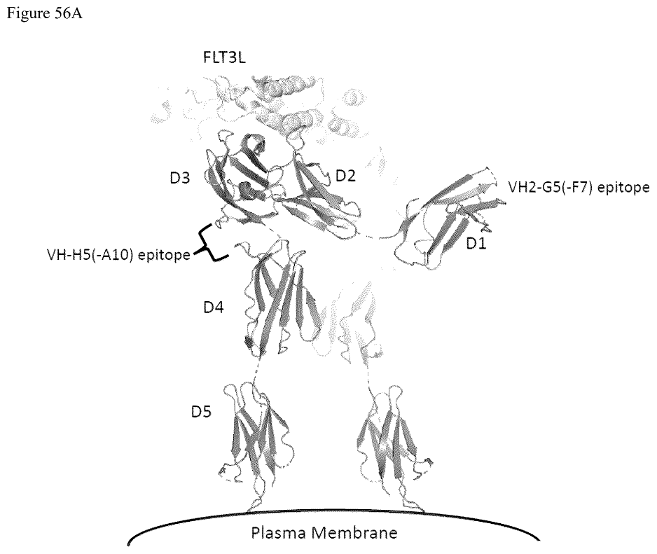

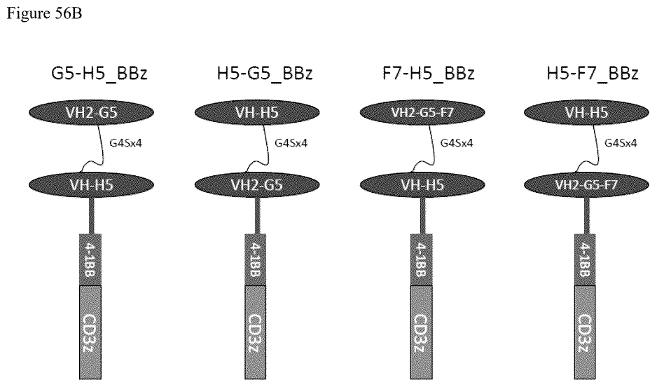

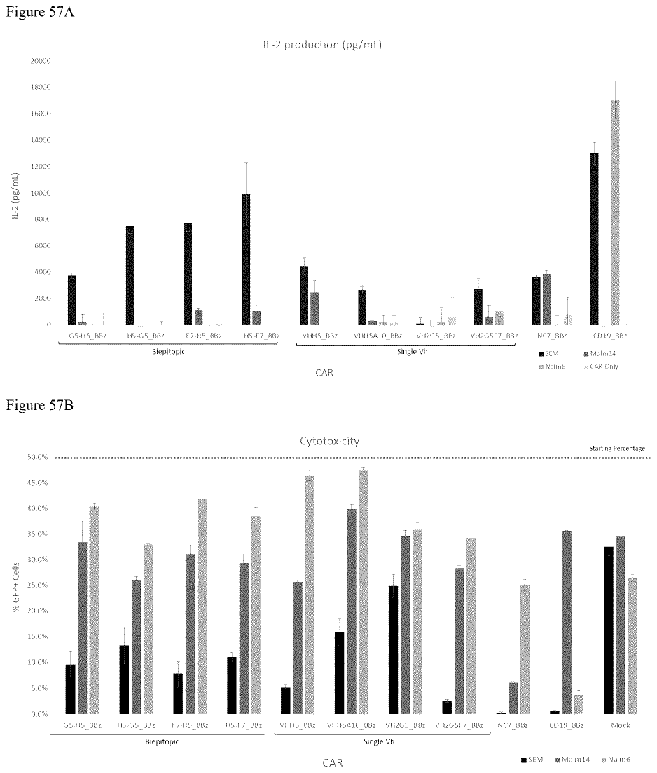

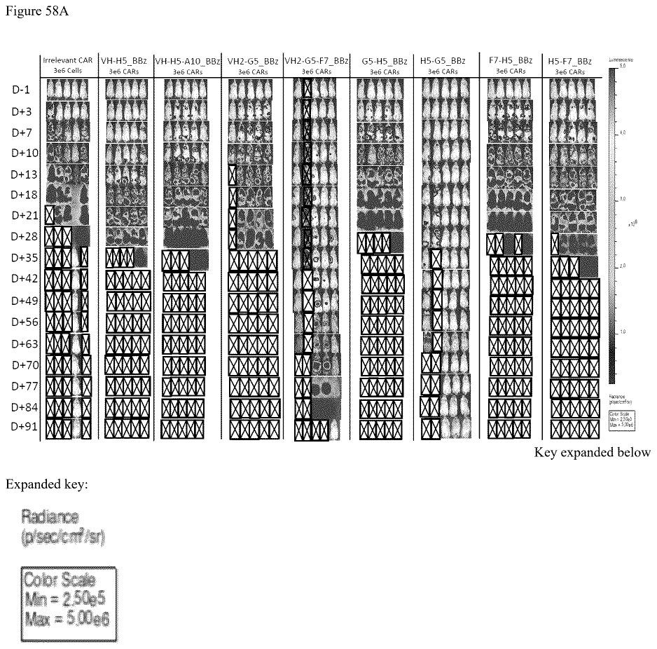

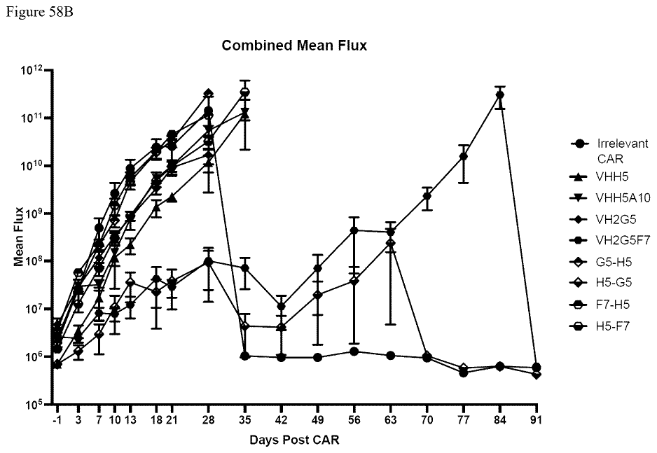

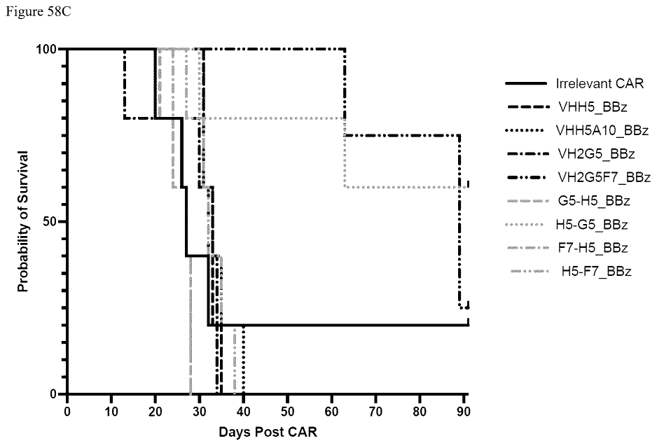

Clones #1-#4 also were used to make CAR clones having the ability to bind to two different epitopes of CD135 (FIG. 56A-B). The in vitro and in vivo activity of cells expressing the mono-paratopic and bi-paratopic CARs was confirmed (FIGS. 57A-B and FIGS. 58A-C).

### Example 3—Designing BiKEs from Binders Having the Ability to Bind to a Human CD135 Polypeptide

Clones #1-#4 are used to make BiKEs having the following configuration: VH Domain of any one of Clones #1-#4⁺ (G₄S) linker (SEQ ID NO: 90)+Anti-NKG2A VH+linker+Anti-NKG2A VL. See, e.g., FIGS. 31-34.

Such BiKEs are used to direct NK cells to kill cells expressing human CD135 such as CD135⁺ cancer cells.

### Example 4—Designing BiTEs from Binders Having the Ability to Bind to a Human CD135 Polypeptide

Clones #1-#4 are used to make BiTEs having the following configuration: VH Domain of any one of Clones #1-#4⁺ (GSGSS)₃ linker (SEQ ID NO: 371)+Anti-CD3 scFv+GSGSSG linker (SEQ ID NO: 372)+hIgG1 Fc. See, e.g., FIGS. 35-38.

Such BiTEs are used to direct T cells to kill cells expressing human CD135 such as CD135⁺ cancer cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

2-12. (canceled)

13. An antigen binding fragment comprising:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

14-24. (canceled)

25. An antibody domain comprising:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

26-36. (canceled)

37. A chimeric antigen receptor comprising an antigen binding domain, a hinge, a transmembrane domain, and one or more signaling domains, wherein said antigen binding domain comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

38. The chimeric antigen receptor of claim 37, wherein said antigen binding domain comprises a VH domain having the ability to bind to a CD135 polypeptide.

39. The chimeric antigen receptor of claim 37, wherein said hinge comprises a hinge set forth in FIG. 17.

40. The chimeric antigen receptor of claim 37, wherein said transmembrane domain comprises a transmembrane domain set forth in FIG. 18.

41. The chimeric antigen receptor of claim 37, wherein said chimeric antigen receptor comprises one or more signaling domains set forth in FIG. 19.

42. A cell comprising a chimeric antigen receptor comprising an antigen binding domain, a hinge, a transmembrane domain, and one or more signaling domains, wherein said antigen binding domain comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEO ID NO:25, SEO ID NO:26, and SEO ID NO:27.

43. The cell of claim 42, wherein said cell is a T cell, a stem cell, or an NK cell.

44. A cell engager comprising a first antigen binding domain, a linker, and a second antigen binding domain, wherein said first antigen binding domain comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

45-67. (canceled)

68. An antibody-drug conjugate (ADC) comprising an antigen binging domain covalently linked to a drug, wherein said antigen binging domain comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEO ID NO:25, SEO ID NO:26, and SEO ID NO:27.

69-70. (canceled)

71. A composition comprising an antibody, an antigen binding fragment, an antibody domain, a cell engager, a cell comprising a chimeric antigen receptor, or an ADC, wherein said antibody, said antigen binding fragment, said antibody domain, said cell engager, said chimeric antigen receptor, and said ADC comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

72-75. (canceled)

76. The composition of claim 71, wherein said composition comprises said cell.

77. (canceled)

78. The composition of claim 71, wherein said composition comprises a checkpoint inhibitor.

79. (canceled)

80. A method of treating a mammal having cancer, wherein said method comprises administering, to said mammal, a composition comprising an antibody, an antigen binding fragment, an antibody domain, a cell engager, a cell comprising a chimeric antigen receptor, or an ADC, wherein said antibody, said antigen binding fragment, said antibody domain, said cell engager, said chimeric antigen receptor, and said ADC comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEQ ID NO:17, SEQ ID NO:18, and SEQ ID NO:19; or
(iv) a heavy chain variable domain or region comprising the amino acid sequences set forth in SEQ ID NO:25, SEQ ID NO:26, and SEQ ID NO:27.

81-95. (canceled)

96. A method for binding a binding molecule to a CD135 polypeptide, wherein said method comprises contacting said CD135 polypeptide with an antibody, an antigen binding fragment, an antibody domain, a chimeric antigen receptor, a cell engager, or an ADC, wherein said antibody, said antigen binding fragment, said antibody domain, said cell engager, said chimeric antigen receptor, and said ADC comprises:
(i) a heavy chain variable domain or region comprising the amino acid sequences set forth in SEQ ID NO:1, SEQ ID NO:2, and SEQ ID NO:3;
(ii) a heavy chain variable domain or region comprising the amino acid sequences set forth in SEQ ID NO:9, SEQ ID NO:10, and SEQ ID NO:11;
(iii) a heavy chain variable domain or region comprising the amino acid sequences set forth in SEO ID NO:17, SEO ID NO:18, and SEO ID NO:19; or
(iv) a heavy chain variable domain or region comprising the amino acid sequences set forth in SEQ ID NO:25, SEQ ID NO:26, and SEQ ID NO:27.

97-100. (canceled)

101. The chimeric antigen receptor of claim 37, wherein said chimeric antigen receptor comprise two or more antigen binding domains.

102. The chimeric antigen receptor of claim 101, wherein said chimeric antigen receptor is a biparatopic chimeric antigen receptor.

103. The chimeric antigen receptor of claim 102, wherein each paratope of said biparatopic chimeric antigen receptor binds to a different epitope of a CD135 polypeptide.


SEQUENCE LISTING (379 sequences)

SEQ ID NO: 1 - protein, 5 aa, synthetic construct
DYEMS

SEQ ID NO: 2 - protein, 17 aa, synthetic construct
SISSSGSTIYYADSVKG

SEQ ID NO: 3 - protein, 10 aa, synthetic construct
AVVGRKYFQH

SEQ ID NO: 4 - protein, 30 aa, synthetic construct
EVQLVESGGGLVQPGGSLRLSCAASAFYFT

SEQ ID NO: 5 - protein, 14 aa, synthetic construct
WVRQAPGQGLEWVS

SEQ ID NO: 6 - protein, 32 aa, synthetic construct
RFTISRDNSKNTLYLQMNSLRAEDTAMYYCAR

SEQ ID NO: 7 - protein, 11 aa, synthetic construct
WGQGTLVTVSS

SEQ ID NO: 8 - protein, 119 aa, synthetic construct
EVQLVESGGGLVQPGGSLRLSCAASAFYFTDYEMSWVRQAPGQGLEWVSSISSSGSTIYYADSVKGRFTISRDNSKNTLYLQMNSLRAEDTAMYYCARAVVGRKYFQHWGQGTLVTVSS

SEQ ID NO: 9 - protein, 5 aa, synthetic construct
DYEMS

SEQ ID NO: 10 - protein, 17 aa, synthetic construct
SISSSGSTIYYADSVKG

SEQ ID NO: 11 - protein, 10 aa, synthetic construct
AVVGRKYFQH

SEQ ID NO: 12 - protein, 30 aa, synthetic construct; identical to SEQ ID NO: 4

SEQ ID NO: 13 - protein, 14 aa, synthetic construct
WVRQAPGQGLERVS

SEQ ID NO: 14 - protein, 32 aa, synthetic construct
RFTISRDNSKNTLYLEMNSLRAEDTAMYYCAR

SEQ ID NO: 15 - protein, 11 aa, synthetic construct
WGQGTLVTVSS

SEQ ID NO: 16 - protein, 119 aa, synthetic construct
EVQLVESGGGLVQPGGSLRLSCAASAFYFTDYEMSWVRQAPGQGLERVSSISSSGSTIYYADSVKGRFTISRDNSKNTLYLEMNSLRAEDTAMYYCARAVVGRKYFQHWGQGTLVTVSS

SEQ ID NO: 17 - protein, 5 aa, synthetic construct
SYEMS

SEQ ID NO: 18 - protein, 17 aa, synthetic construct
YISSSGSTIYYADSVKG

SEQ ID NO: 19 - protein, 9 aa, synthetic construct
NPAPGTADQ

SEQ ID NO: 20 - protein, 30 aa, synthetic construct
EVQLVESGGGLVQPGGSLRLSCAASYFDFD

SEQ ID NO: 21 - protein, 14 aa, synthetic construct
WVRQAPGQGLEWVS

SEQ ID NO: 22 - protein, 32 aa, synthetic construct
RFTISRDNSKNTLYLQMNSLRAEDTAVYYCAR

SEQ ID NO: 23 - protein, 11 aa, synthetic construct
WGQGTLVTVSS

SEQ ID NO: 24 - protein, 118 aa, synthetic construct
EVQLVESGGGLVQPGGSLRLSCAASYFDFDSYEMSWVRQAPGQGLEWVSYISSSGSTIYYADSVKGRFTISRDNSKNTLYLQMNSLRAEDTAVYYCARNPAPGTADQWGQGTLVTVSS

SEQ ID NO: 25 - protein, 5 aa, synthetic construct
SYEMS

SEQ ID NO: 26 - protein, 17 aa, synthetic construct
YISSSGSTIYYADSVKG

SEQ ID NO: 27 - protein, 9 aa, synthetic construct
NPAPGTVDQ

SEQ ID NO: 28 - protein, 30 aa, synthetic construct
EVQLVESGGGLVQPGGSLRLSCAASYLDFD

SEQ ID NO: 29 - protein, 14 aa, synthetic construct
WFRQAPGQGLEWVS

SEQ ID NO: 30 - protein, 32 aa, synthetic construct
RFTISRDNSQNTLYLQMNSLRAEDTAVYYCAR

SEQ ID NO: 31 - protein, 11 aa, synthetic construct
WGQGTLVTVSS

SEQ ID NO: 32 - protein, 118 aa, synthetic construct
EVQLVESGGGLVQPGGSLRLSCAASYLDFDSYEMSWFRQAPGQGLEWVSYISSSGSTIYYADSVKGRFTISRDNSQNTLYLQMNSLRAEDTAVYYCARNPAPGTVDQWGQGTLVTVSS

SEQ ID NO: 33 - DNA, 357 bp, synthetic construct
  source 1..357: mol_type = other DNA
gaggtgcagctggtggagtctgggggaggcttggtacagcctggagggtccctgagactctcctgtgcagcctctgccttctatttcactgattatgaaatgagctgggtccgccaggctccaggacaagggcttgagtgggtctcatccattagtagtagtggtagtaccatatactacgcagactctgtgaagggccgattcaccatctccagagacaattccaagaacacgctgtatctgcaaatgaacagcctgagagccgaggacacagccatgtattactgtgcgagagccgtggttggccgcaaatacttccagcactggggccagggcaccctggtcaccgtctcctca

SEQ ID NO: 34 - DNA, 357 bp, synthetic construct
  source 1..357: mol_type = other DNA
gaggtgcagctggtggagtctgggggaggcttggtacagcctggagggtccctaagactctcctgtgcagcctctgccttctatttcactgattatgaaatgagctgggtccgccaggctccaggacaagggcttgagagggtctcatccattagtagtagtggtagtaccatatactacgcagactctgtgaagggccgattcactatctccagagacaattcgaagaacacgctgtatctggaaatgaacagcctgagagccgaggacacagccatgtattactgtgcgagagccgtggttggccgcaaatacttccagcactggggccagggcaccctggtcaccgtctcctca

SEQ ID NO: 35 - DNA, 354 bp, synthetic construct
  source 1..354: mol_type = other DNA
gaggtgcagctggtggagtctgggggaggcttggtacagcctggagggtccctgagactctcctgtgcagcctcttatttcgatttcgattcttatgaaatgagctgggtccgccaggctccaggacaaggccttgagtgggtttcatacattagtagtagtggtagtaccatatactacgcagactccgtgaagggccgattcaccatctccagagacaattccaagaacacgctgtatctgcaaatgaacagcctgagagccgaggacacagctgtatattactgtgcgagaaatcccgcgcctggtacggcggaccagtggggccagggaacgctggtcaccgtctcctca

SEQ ID NO: 36 - DNA, 354 bp, synthetic construct
  source 1..354: mol_type = other DNA
gaggtgcagctggtggagtctggtggaggcttggtacagcctggagggtccctgagactctcctgtgcagcctcttatctcgatttcgattcttatgaaatgagctggttccgccaggctccaggacaaggccttgagtgggtttcatacattagtagtagtggtagtaccatatactacgcagactccgtgaagggccgattcaccatctccagagacaattcccagaacacgctgtatctgcaaatgaacagcctgagagccgaggacacagctgtatattactgtgcgagaaatcccgcgccaggtacggtggaccagtggggccagggaacgctggtcaccgtctcctca

SEQ ID NO: 37 - protein, 952 aa, Homo sapiens [sequence omitted]

SEQ ID NO: 38 - DNA, 2859 bp, Homo sapiens
  source 1..2859: mol_type = other DNA
atgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagaactcagggacagtgtacgaagctgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggatggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcggcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtag

SEQ ID NO: 39 - protein, 517 aa, Homo sapiens
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNIS

SEQ ID NO: 40 - DNA, 1551 bp, Homo sapiens
  source 1..1551: mol_type = other DNA
aatcaagatctgcctgtgatcaagtgtgttttaatcaatcataagaacaatgattcatcagtggggaagtcatcatcatatcccatggtatcagaatccccggaagacctcgggtgtgcgttgagaccccagaactcagggacagtgtacgaagctgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggatggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctca

SEQ ID NO: 41 - protein, 15 aa, Homo sapiens
EPKSCDKTHTCPPCP

SEQ ID NO: 42 - DNA, 45 bp, Homo sapiens
  source 1..45: mol_type = other DNA
gagcccaaatcttgtgacaaaactcacacatgcccaccgtgccca

SEQ ID NO: 43 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 651 bp, Homo sapiens
  source 1..651: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 45-46 - skipped (2 entries)

SEQ ID NO: 47 - protein, 23 aa, synthetic construct
DIQMTQSPSSLSASVGDRVTITC

SEQ ID NO: 48 - protein, 15 aa, synthetic construct
WYQQKPGKAPKLLIY

SEQ ID NO: 49 - protein, 32 aa, synthetic construct
GVPSRFSGSGSGTDFTLTISSLQPEDFGVYYC

SEQ ID NO: 50 - protein, 11 aa, synthetic construct
FGQGTRLEIKR

SEQ ID NO: 51-52 - skipped (2 entries)

SEQ ID NO: 53 - protein, 25 aa, synthetic construct
QSALTQPASVSGSPGQSITISCTRA

SEQ ID NO: 54 - protein, 17 aa, synthetic construct
VSWYQQHPGKAPKLMIY

SEQ ID NO: 55 - protein, 36 aa, synthetic construct
NRPSGVSNRFSGSKSGNTASLTISGLQAEDEADYYC

SEQ ID NO: 56 - protein, 11 aa, synthetic construct
FGGGTQLTVLG

SEQ ID NO: 57-58 - skipped (2 entries)

SEQ ID NO: 59 - protein, 23 aa, synthetic construct
DIQMTQSPSSLSASVGDRVTITC

SEQ ID NO: 60 - protein, 15 aa, synthetic construct
WYQQKPGKAPKLLIY

SEQ ID NO: 61 - protein, 32 aa, synthetic construct; identical to SEQ ID NO: 49

SEQ ID NO: 62 - protein, 11 aa, synthetic construct
FGQGTRLEIKR

SEQ ID NO: 63-64 - skipped (2 entries)

SEQ ID NO: 65 - protein, 23 aa, synthetic construct
DIQMTQSPSSLSASVGDRVTITC

SEQ ID NO: 66 - protein, 15 aa, synthetic construct
WYQQKPGKAPKLLIY

SEQ ID NO: 67 - protein, 32 aa, synthetic construct
GVPSRFSGSGSGTDFTLTISSLQPEDVGVYYC

SEQ ID NO: 68 - protein, 11 aa, synthetic construct
FGGGTKLEIKR

SEQ ID NO: 69 - protein, 15 aa, synthetic construct
GGGGSGGSGSGGGGS

SEQ ID NO: 70 - protein, 15 aa, synthetic construct
GGGGSGGSGSSGGGS

SEQ ID NO: 71 - protein, 15 aa, synthetic construct
GGGGSGGGGSGGGGS

SEQ ID NO: 72 - protein, 15 aa, synthetic construct
GGGGSGSGASSGGGS

SEQ ID NO: 73 - protein, 15 aa, synthetic construct
GGGGSGSGASGGGGS

SEQ ID NO: 74 - protein, 15 aa, synthetic construct
SGGGSGGGGSGGGGS

SEQ ID NO: 75 - protein, 18 aa, synthetic construct
GSTSGSGKPGSGEGSTKG

SEQ ID NO: 76 - protein, 19 aa, synthetic construct
PNGASQSSSASHTGSAPGS

SEQ ID NO: 77 - protein, 19 aa, synthetic construct
PNGASNSGSAPDTSSAPGS

SEQ ID NO: 78 - protein, 19 aa, synthetic construct
PNGASHSGSAPNTSSAPGS

SEQ ID NO: 79 - protein, 19 aa, synthetic construct
PNGASESGSASKTSSASGS

SEQ ID NO: 80 - protein, 19 aa, synthetic construct
PNGASNSGSAPKTGSASGS

SEQ ID NO: 81 - protein, 19 aa, synthetic construct
PNGASKSGSASQTSSAPGS

SEQ ID NO: 82 - protein, 19 aa, synthetic construct
PNGASHSSSASQTGSAPGS

SEQ ID NO: 83 - protein, 12 aa, synthetic construct
PNGASKRSAPGS

SEQ ID NO: 84 - protein, 19 aa, synthetic construct
PNGASHSGSAPHTSSASGS

SEQ ID NO: 85 - protein, 15 aa, synthetic construct
RGRGRGRGRSRGGGS

SEQ ID NO: 86 - protein, 15 aa, synthetic construct
SHGGSHGGGSGGGGS

SEQ ID NO: 87 - protein, 5 aa, synthetic construct
GQAGR

SEQ ID NO: 88 - protein, 8 aa, synthetic construct
GGGSGGGG

SEQ ID NO: 89 - protein, 5 aa, synthetic construct
SGGGG

SEQ ID NO: 90 - protein, 5 aa, synthetic construct
GGGGS

SEQ ID NO: 91 - protein, 30 aa, synthetic construct
GGGGSGGGGSGGGGSGGGGSGGGGSGGGGS

SEQ ID NO: 92 - protein, 16 aa, synthetic construct
GGGGGSGGGGSGGGGS

SEQ ID NO: 93 - protein, 26 aa, synthetic construct
GGGGGSGGGGSGGGGSGGGGSGGGGS

SEQ ID NO: 94 - protein, 25 aa, synthetic construct
GGGGSGGGGSGGGGSGGGGSGGGGS

SEQ ID NO: 95 - skipped

SEQ ID NO: 96 - protein, 18 aa, synthetic construct
VEGGSGGSGGSGGSGGVD

SEQ ID NO: 97 - protein, 22 aa, synthetic construct
MDMRVPAQLLGLLLLWLRGARC

SEQ ID NO: 98 - DNA, 66 bp, synthetic construct
  source 1..66: mol_type = other DNA
atggatatgagggtccccgcacaattgctcggtctccttctgctttggctccggggcgcgcggtgc

SEQ ID NO: 99 - protein, 22 aa, synthetic construct
MDMRVLAQLLGLLLLCFPGARC

SEQ ID NO: 100 - protein, 20 aa, synthetic construct
MEAPAQLLFLLLLWLPDTTG

SEQ ID NO: 101 - protein, 20 aa, synthetic construct
MVLQTQVFISLLLWISGAYG

SEQ ID NO: 102 - protein, 19 aa, synthetic construct
MLPSQLIGFLLLWVPASRG

SEQ ID NO: 103 - protein, 22 aa, synthetic construct
MDMRVPAQLLGLLLLWLSGARC

SEQ ID NO: 104 - protein, 15 aa, synthetic construct
EPKSCDKTHTCPPCP

SEQ ID NO: 105 - protein, 15 aa, synthetic construct
DKTHTCPPCPAPPVA

SEQ ID NO: 106 - protein, 12 aa, synthetic construct
ESKYGPPCPPCP

SEQ ID NO: 107 - protein, 12 aa, synthetic construct
ESKYGPPCPSCP

SEQ ID NO: 108 - protein, 49 aa, synthetic construct
KPTTTPAPRPPTPAPTIASQPLSLRPEACRPAAGGAVHTRGLDFACDIY

SEQ ID NO: 109 - protein, 47 aa, synthetic construct
TTTPAPRPPTPAPTIASQPLSLRPEACRPAAGGAVHTRGLDFACDIY

SEQ ID NO: 110 - protein, 45 aa, synthetic construct
TTTPAPRPPTPAPTIASQPLSLRPEACRPAAGGAVHTRGLDFACD

SEQ ID NO: 111 - protein, 39 aa, synthetic construct
IEVMYPPPYLDNERSNGTIIHVKGKHLCPSPLFPGPSKP

SEQ ID NO: 112 - protein, 21 aa, synthetic construct
LCYLLDGILFIYGVILTALFL

SEQ ID NO: 113 - protein, 22 aa, synthetic construct
MALIVLGGVAGLLLFIGLGIFF

SEQ ID NO: 114 - protein, 21 aa, synthetic construct
IYIWAPLAGTCGVLLLSLVIT

SEQ ID NO: 115 - protein, 24 aa, synthetic construct
IYIWAPLAGTCGVLLLSLVITLYC

SEQ ID NO: 116 - protein, 22 aa, synthetic construct
IWAPLAGTCGVLLLSLVITLYC

SEQ ID NO: 117 - protein, 19 aa, synthetic construct
IWAPLAGTCGVLLLSLVIT

SEQ ID NO: 118 - protein, 27 aa, synthetic construct
FWVLVVVGGVLACYSLLVTVAFIIFWV

SEQ ID NO: 119 - protein, 21 aa, synthetic construct
FWLPIGCAAFVVVCILGCILI

SEQ ID NO: 120 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 121 - protein, 113 aa, synthetic construct
RVKFSRSADAPAYKQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 122 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 42 aa, synthetic construct
KRGRKKLLYIFKQPFMRPVQTTQEEDGCSCRFPEEEEGGCEL

SEQ ID NO: 124 - protein, 37 aa, synthetic construct
KRGRKKLLYIFKQPFMRPVQTTQEEDGCSCRFPEEEE

SEQ ID NO: 125 - protein, 41 aa, synthetic construct
RSKRSRLLHSDYMNMTPRRPGPTRKHYQPYAPPRDFAAYRS

SEQ ID NO: 126 - protein, 42 aa, synthetic construct
ALYLLRRDQRLPPDAHKPPGGGSFRTPIQEEQADAHSTLAKI

SEQ ID NO: 127 - protein, 38 aa, synthetic construct
CWLTKKKYSSSVHDPNGEYMFMRAVNTAKKSRLTDVTL

SEQ ID NO: 128 - protein, 24 aa, synthetic construct
LCARPRRSPAQEDGKVYINMPGRG

SEQ ID NO: 129 - protein, 52 aa, synthetic construct
YFLGRLVPRGRGAAEAATRKQRITETESPYQELQGQRSDVYSDLNTQRPYYK

SEQ ID NO: 130 - protein, 48 aa, synthetic construct
QRRKYRSNKGESPVEPAEPCHYSCPREEEGSTIPIQEDYRKPEPACSP

SEQ ID NO: 131 - protein, 376 aa, synthetic construct
MALPVTALLLPLALLLHAARPDYKDDDDKEVQLVESGGGLVQPGGSLRLSCAASAFYFTDYEMSWVRQAPGQGLEWVSSISSSGSTIYYADSVKGRFTISRDNSKNTLYLQMNSLRAEDTAMYYCARAVVGRKYFQHWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 132 - DNA, 63 bp, synthetic construct
  source 1..63: mol_type = other DNA
atggctctgcctgttacagctctgctgctgcctctggctctgcttctgcatgccgccagacct

SEQ ID NO: 133 - DNA, 24 bp, synthetic construct
  source 1..24: mol_type = other DNA
gactacaaggacgacgacgacaaa

SEQ ID NO: 134 - DNA, 357 bp, synthetic construct
  source 1..357: mol_type = other DNA
gaggtgcagctggtcgaatctggcggaggacttgttcagcctggcggctctctgagactgtcttgtgccgccagcgccttctacttcaccgactacgagatgagctgggtccgacaggctcctggacaaggcttggaatgggtgtccagcatcagcagcagcggcagcacaatctactacgccgacagcgtgaagggcagattcaccatcagccgggacaacagcaagaacaccctgtacctgcagatgaacagcctgagagccgaggacaccgccatgtactattgtgccagagccgtcgtgggcagaaagtacttccagcactggggccagggcaccctggttacagtttcttct

SEQ ID NO: 135 - skipped

SEQ ID NO: 136 - DNA, 135 bp, synthetic construct
  source 1..135: mol_type = other DNA
accaccacaccagctcctcggccacctactccagctccaacaattgccagccagcctctgtctctgaggcccgaagcttgtagacctgctgctggcggagccgtgcatacaagaggactggatttcgcctgcgac

SEQ ID NO: 137 - DNA, 72 bp, synthetic construct
  source 1..72: mol_type = other DNA
atctacatctgggcccctctggctggaacatgtggcgtgctgctgctcagcctggtcatcaccctgtactgc

SEQ ID NO: 138 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 139-141 - skipped (3 entries)

SEQ ID NO: 142 - protein, 376 aa, synthetic construct
MALPVTALLLPLALLLHAARPDYKDDDDKEVQLVESGGGLVQPGGSLRLSCAASAFYFTDYEMSWVRQAPGQGLERVSSISSSGSTIYYADSVKGRFTISRDNSKNTLYLEMNSLRAEDTAMYYCARAVVGRKYFQHWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 143 - DNA, 63 bp, synthetic construct; identical to SEQ ID NO: 132
  source 1..63: mol_type = other DNA

SEQ ID NO: 144 - DNA, 24 bp, synthetic construct
  source 1..24: mol_type = other DNA
gactacaaggacgacgacgacaaa

SEQ ID NO: 145 - DNA, 357 bp, synthetic construct
  source 1..357: mol_type = other DNA
gaggtgcagctggtcgaatctggcggaggacttgttcagcctggcggctctctgagactgtcttgtgccgccagcgccttctacttcaccgactacgagatgagctgggtccgacaggctcctggacagggccttgaaagagtgtccagcatcagcagcagcggcagcaccatctactacgccgatagcgtgaagggcagattcaccatcagccgggacaacagcaagaacaccctgtacctggaaatgaacagcctgagagccgaggacaccgccatgtactattgtgccagagccgtcgtgggcagaaagtacttccagcactggggccagggcaccctggttacagtttcttct

SEQ ID NO: 146 - skipped

SEQ ID NO: 147 - DNA, 135 bp, synthetic construct; identical to SEQ ID NO: 136
  source 1..135: mol_type = other DNA

SEQ ID NO: 148 - DNA, 72 bp, synthetic construct; identical to SEQ ID NO: 137
  source 1..72: mol_type = other DNA

SEQ ID NO: 149 - DNA, 126 bp, synthetic construct; identical to SEQ ID NO: 138
  source 1..126: mol_type = other DNA

SEQ ID NO: 150 - skipped

SEQ ID NO: 151 - DNA, 333 bp, synthetic construct
  source 1..333: mol_type = other DNA
gtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaaga

SEQ ID NO: 152-153 - skipped (2 entries)

SEQ ID NO: 154 - protein, 375 aa, synthetic construct
MALPVTALLLPLALLLHAARPDYKDDDDKEVQLVESGGGLVQPGGSLRLSCAASYFDFDSYEMSWVRQAPGQGLEWVSYISSSGSTIYYADSVKGRFTISRDNSKNTLYLQMNSLRAEDTAVYYCARNPAPGTADQWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 155 - DNA, 63 bp, synthetic construct; identical to SEQ ID NO: 132
  source 1..63: mol_type = other DNA

SEQ ID NO: 156 - DNA, 24 bp, synthetic construct
  source 1..24: mol_type = other DNA
gactacaaggacgacgacgacaaa

SEQ ID NO: 157 - DNA, 354 bp, synthetic construct
  source 1..354: mol_type = other DNA
gaggtgcagctggtcgaatctggcggaggacttgttcagcctggcggctctctgagactgtcttgtgccgccagctacttcgacttcgacagctacgagatgagctgggtccgacaggctcctggacaaggcttggagtgggtgtcctacatcagcagcagcggcagcaccatctactacgccgatagcgtgaagggcagattcaccatcagccgggacaacagcaagaacaccctgtacctgcagatgaacagcctgagagccgaggacaccgccgtgtactactgcgccagaaatcctgctcctggcaccgctgatcaatggggccagggaacactggtcaccgtgtcatct

SEQ ID NO: 158 - skipped

SEQ ID NO: 159 - DNA, 135 bp, synthetic construct; identical to SEQ ID NO: 136
  source 1..135: mol_type = other DNA

SEQ ID NO: 160 - DNA, 72 bp, synthetic construct; identical to SEQ ID NO: 137
  source 1..72: mol_type = other DNA

SEQ ID NO: 161 - DNA, 126 bp, synthetic construct; identical to SEQ ID NO: 138
  source 1..126: mol_type = other DNA

SEQ ID NO: 162 - skipped

SEQ ID NO: 163 - DNA, 333 bp, synthetic construct; identical to SEQ ID NO: 151
  source 1..333: mol_type = other DNA

SEQ ID NO: 164-165 - skipped (2 entries)

SEQ ID NO: 166 - protein, 375 aa, synthetic construct
MALPVTALLLPLALLLHAARPDYKDDDDKEVQLVESGGGLVQPGGSLRLSCAASYLDFDSYEMSWFRQAPGQGLEWVSYISSSGSTIYYADSVKGRFTISRDNSQNTLYLQMNSLRAEDTAVYYCARNPAPGTVDQWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 167 - DNA, 63 bp, synthetic construct; identical to SEQ ID NO: 132
  source 1..63: mol_type = other DNA

SEQ ID NO: 168 - DNA, 24 bp, synthetic construct
  source 1..24: mol_type = other DNA
gactacaaggacgacgacgacaaa

SEQ ID NO: 169 - DNA, 354 bp, synthetic construct
  source 1..354: mol_type = other DNA
gaggtgcagctggtcgaatctggcggaggacttgttcagcctggcggctctctgagactgtcttgtgccgccagctacctggacttcgacagctacgagatgagctggttcagacaggcccctggacagggccttgagtgggtgtcctacatcagcagcagcggcagcaccatctactacgccgatagcgtgaagggcagattcaccatcagccgggacaacagccagaacaccctgtacctgcagatgaacagcctgagagccgaggacaccgccgtgtactactgcgccagaaatcctgctcctggcaccgtggatcaatggggccagggaacactggtcaccgtgtcatct

SEQ ID NO: 170 - skipped

SEQ ID NO: 171 - DNA, 135 bp, synthetic construct; identical to SEQ ID NO: 136
  source 1..135: mol_type = other DNA

SEQ ID NO: 172 - DNA, 72 bp, synthetic construct; identical to SEQ ID NO: 137
  source 1..72: mol_type = other DNA

SEQ ID NO: 173 - DNA, 126 bp, synthetic construct; identical to SEQ ID NO: 138
  source 1..126: mol_type = other DNA

SEQ ID NO: 174 - skipped

SEQ ID NO: 175 - DNA, 333 bp, synthetic construct; identical to SEQ ID NO: 151
  source 1..333: mol_type = other DNA

SEQ ID NO: 176-177 - skipped (2 entries)

SEQ ID NO: 178 - protein, 375 aa, synthetic construct
MALPVTALLLPLALLLHAARPDYKDDDDKEVQLVESGGGLVQPGGSLRLSCAASAFYFTDYEMSWVRQAPGQGLEWVSSISSSGSTIYYADSVKGRFTISRDNSKNTLYLQMNSLRAEDTAMYYCARAVVGRKYFQHWGQGTLVTVSSTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 179 - DNA, 63 bp, synthetic construct; identical to SEQ ID NO: 132
  source 1..63: mol_type = other DNA

SEQ ID NO: 180 - DNA, 24 bp, synthetic construct
  source 1..24: mol_type = other DNA
gactacaaggacgacgacgacaaa

SEQ ID NO: 181 - DNA, 357 bp, synthetic construct; identical to SEQ ID NO: 134
  source 1..357: mol_type = other DNA

SEQ ID NO: 182 - skipped

SEQ ID NO: 183 - DNA, 330 bp, synthetic construct
  source 1..330: mol_type = other DNA
ggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccgg

SEQ ID NO: 184 - skipped

SEQ ID NO: 185 - DNA, 333 bp, synthetic construct
  source 1..333: mol_type = other DNA
gtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 186-187 - skipped (2 entries)

SEQ ID NO: 188 - protein, 375 aa, synthetic construct
MALPVTALLLPLALLLHAARPDYKDDDDKEVQLVESGGGLVQPGGSLRLSCAASAFYFTDYEMSWVRQAPGQGLERVSSISSSGSTIYYADSVKGRFTISRDNSKNTLYLEMNSLRAEDTAMYYCARAVVGRKYFQHWGQGTLVTVSSTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 189 - DNA, 63 bp, synthetic construct; identical to SEQ ID NO: 132
  source 1..63: mol_type = other DNA

SEQ ID NO: 190 - DNA, 24 bp, synthetic construct
  source 1..24: mol_type = other DNA
gactacaaggacgacgacgacaaa

SEQ ID NO: 191 - DNA, 357 bp, synthetic construct; identical to SEQ ID NO: 145
  source 1..357: mol_type = other DNA

SEQ ID NO: 192 - skipped

SEQ ID NO: 193 - DNA, 330 bp, synthetic construct; identical to SEQ ID NO: 183
  source 1..330: mol_type = other DNA

SEQ ID NO: 194 - skipped

SEQ ID NO: 195 - DNA, 333 bp, synthetic construct; identical to SEQ ID NO: 185
  source 1..333: mol_type = other DNA

SEQ ID NO: 196-197 - skipped (2 entries)

SEQ ID NO: 198 - protein, 374 aa, synthetic construct
MALPVTALLLPLALLLHAARPDYKDDDDKEVQLVESGGGLVQPGGSLRLSCAASYFDFDSYEMSWVRQAPGQGLEWVSYISSSGSTIYYADSVKGRFTISRDNSKNTLYLQMNSLRAEDTAVYYCARNPAPGTADQWGQGTLVTVSSTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 199 - DNA, 63 bp, synthetic construct; identical to SEQ ID NO: 132
  source 1..63: mol_type = other DNA

SEQ ID NO: 200 - DNA, 24 bp, synthetic construct
  source 1..24: mol_type = other DNA
gactacaaggacgacgacgacaaa

SEQ ID NO: 201 - DNA, 354 bp, synthetic construct; identical to SEQ ID NO: 157
  source 1..354: mol_type = other DNA

SEQ ID NO: 202 - skipped

SEQ ID NO: 203 - DNA, 330 bp, synthetic construct; identical to SEQ ID NO: 183
  source 1..330: mol_type = other DNA

SEQ ID NO: 204 - skipped

SEQ ID NO: 205 - DNA, 333 bp, synthetic construct; identical to SEQ ID NO: 185
  source 1..333: mol_type = other DNA

SEQ ID NO: 206-207 - skipped (2 entries)

SEQ ID NO: 208 - protein, 374 aa, synthetic construct
MALPVTALLLPLALLLHAARPDYKDDDDKEVQLVESGGGLVQPGGSLRLSCAASYLDFDSYEMSWFRQAPGQGLEWVSYISSSGSTIYYADSVKGRFTISRDNSQNTLYLQMNSLRAEDTAVYYCARNPAPGTVDQWGQGTLVTVSSTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 209 - DNA, 63 bp, synthetic construct; identical to SEQ ID NO: 132
  source 1..63: mol_type = other DNA

SEQ ID NO: 210 - DNA, 24 bp, synthetic construct
  source 1..24: mol_type = other DNA
gactacaaggacgacgacgacaaa

SEQ ID NO: 211 - DNA, 354 bp, synthetic construct; identical to SEQ ID NO: 169
  source 1..354: mol_type = other DNA

SEQ ID NO: 212 - skipped

SEQ ID NO: 213 - DNA, 330 bp, synthetic construct; identical to SEQ ID NO: 183
  source 1..330: mol_type = other DNA

SEQ ID NO: 214 - skipped

SEQ ID NO: 215 - DNA, 333 bp, synthetic construct; identical to SEQ ID NO: 185
  source 1..333: mol_type = other DNA

SEQ ID NO: 216-217 - skipped (2 entries)

SEQ ID NO: 218 - protein, 271 aa, synthetic construct
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 219 - DNA, 813 bp, synthetic construct
  source 1..813: mol_type = other DNA
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 220 - protein, 119 aa, synthetic construct [sequence omitted]

SEQ ID NO: 221 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 222 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 223 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 224 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 225 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 226 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 227 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 228 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 229 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 230 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 231 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 232 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 233 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 234 - protein, 107 aa, synthetic construct [sequence omitted]

SEQ ID NO: 235 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 236 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 237 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 238 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 239 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 240 - protein, 374 aa, synthetic construct
EVQLVESGGGLVQPGGSLRLSCAASAFYFTDYEMSWVRQAPGQGLEWVSSISSSGSTIYYADSVKGRFTISRDNSKNTLYLQMNSLRAEDTAMYYCARAVVGRKYFQHWGQGTLVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 241 - protein, 374 aa, synthetic construct
EVQLVESGGGLVQPGGSLRLSCAASAFYFTDYEMSWVRQAPGQGLERVSSISSSGSTIYYADSVKGRFTISRDNSKNTLYLEMNSLRAEDTAMYYCARAVVGRKYFQHWGQGTLVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 242 - protein, 373 aa, synthetic construct
EVQLVESGGGLVQPGGSLRLSCAASYFDFDSYEMSWVRQAPGQGLEWVSYISSSGSTIYYADSVKGRFTISRDNSKNTLYLQMNSLRAEDTAVYYCARNPAPGTADQWGQGTLVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 243 - protein, 373 aa, synthetic construct
EVQLVESGGGLVQPGGSLRLSCAASYLDFDSYEMSWFRQAPGQGLEWVSYISSSGSTIYYADSVKGRFTISRDNSQNTLYLQMNSLRAEDTAVYYCARNPAPGTVDQWGQGTLVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 244 - protein, 642 aa, synthetic construct
MTRLTVLALLAGLLASSRAEVQLVESGGGLVQPGGSLRLSCAASAFYFTDYEMSWVRQAPGQGLEWVSSISSSGSTIYYADSVKGRFTISRDNSKNTLYLQMNSLRAEDTAMYYCARAVVGRKYFQHWGQGTLVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 642 aa, synthetic construct
MTRLTVLALLAGLLASSRAEVQLVESGGGLVQPGGSLRLSCAASAFYFTDYEMSWVRQAPGQGLERVSSISSSGSTIYYADSVKGRFTISRDNSKNTLYLEMNSLRAEDTAMYYCARAVVGRKYFQHWGQGTLVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 641 aa, synthetic construct
MTRLTVLALLAGLLASSRAEVQLVESGGGLVQPGGSLRLSCAASYFDFDSYEMSWVRQAPGQGLEWVSYISSSGSTIYYADSVKGRFTISRDNSKNTLYLQMNSLRAEDTAVYYCARNPAPGTADQWGQGTLVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 641 aa, synthetic construct
MTRLTVLALLAGLLASSRAEVQLVESGGGLVQPGGSLRLSCAASYLDFDSYEMSWFRQAPGQGLEWVSYISSSGSTIYYADSVKGRFTISRDNSQNTLYLQMNSLRAEDTAVYYCARNPAPGTVDQWGQGTLVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 8 aa, synthetic construct
EPKSCDKT

SEQ ID NO: 249 - protein, 43 aa, synthetic construct
GAAAIEVMYPPPYLDNEKSNGTIIHVKGKHLCPSPLFPGPSKP

SEQ ID NO: 250 - protein, 5 aa, synthetic construct
EYEMS

SEQ ID NO: 251 - protein, 5 aa, synthetic construct
EYDMS

SEQ ID NO: 252 - protein, 5 aa, synthetic construct
DYDMS

SEQ ID NO: 253 - protein, 5 aa, synthetic construct
EYEMH

SEQ ID NO: 254 - protein, 5 aa, synthetic construct
DYDMH

SEQ ID NO: 255 - protein, 5 aa, synthetic construct
DYEMH

SEQ ID NO: 256 - protein, 5 aa, synthetic construct
EYEMT

SEQ ID NO: 257 - protein, 5 aa, synthetic construct
DYDMT

SEQ ID NO: 258 - protein, 5 aa, synthetic construct
DYEMT

SEQ ID NO: 259 - protein, 5 aa, synthetic construct
EYDMH

SEQ ID NO: 260 - protein, 17 aa, synthetic construct
TISSSGSTIYYADSVKG

SEQ ID NO: 261 - protein, 17 aa, synthetic construct
SITSSGSTIYYADSVKG

SEQ ID NO: 262 - protein, 17 aa, synthetic construct
SISTSGSTIYYADSVKG

SEQ ID NO: 263 - protein, 17 aa, synthetic construct
SISSTGSTIYYADSVKG

SEQ ID NO: 264 - protein, 17 aa, synthetic construct
SISSSGSTIYYAESVKG

SEQ ID NO: 265 - protein, 17 aa, synthetic construct
SISSSGSTIYYADTVKG

SEQ ID NO: 266 - protein, 17 aa, synthetic construct
SISSSGSTIYYADSVRG

SEQ ID NO: 267 - protein, 17 aa, synthetic construct
SISSSGSTIYYADSVRA

SEQ ID NO: 268 - protein, 17 aa, synthetic construct
SISSSGSTIYYADSVKA

SEQ ID NO: 269 - protein, 17 aa, synthetic construct
SLSSSGSTIYYADSVKG

SEQ ID NO: 270 - protein, 10 aa, synthetic construct
AVVGKKYFQH

SEQ ID NO: 271 - protein, 10 aa, synthetic construct
AVVGRKYFNH

SEQ ID NO: 272 - protein, 10 aa, synthetic construct
AVVGKKYFNH

SEQ ID NO: 273 - protein, 10 aa, synthetic construct
AVVARKYFQH

SEQ ID NO: 274 - protein, 10 aa, synthetic construct
GVVGRKYFQH

SEQ ID NO: 275 - protein, 10 aa, synthetic construct
AVAGRKYFQH

SEQ ID NO: 276 - protein, 10 aa, synthetic construct
AAVGRKYFQH

SEQ ID NO: 277 - protein, 10 aa, synthetic construct
AVVGKKYFNH

SEQ ID NO: 278 - protein, 10 aa, synthetic construct
GVVGKKYFQH

SEQ ID NO: 279 - protein, 10 aa, synthetic construct
ALVGRRYFQH

SEQ ID NO: 280 - protein, 5 aa, synthetic construct
EYEMS

SEQ ID NO: 281 - protein, 5 aa, synthetic construct
EYDMS

SEQ ID NO: 282 - protein, 5 aa, synthetic construct
DYDMS

SEQ ID NO: 283 - protein, 5 aa, synthetic construct
EYEMH

SEQ ID NO: 284 - protein, 5 aa, synthetic construct
DYDMH

SEQ ID NO: 285 - protein, 5 aa, synthetic construct
DYEMH

SEQ ID NO: 286 - protein, 5 aa, synthetic construct
EYEMT

SEQ ID NO: 287 - protein, 5 aa, synthetic construct
DYDMT

SEQ ID NO: 288 - protein, 5 aa, synthetic construct
DYEMT

SEQ ID NO: 289 - protein, 5 aa, synthetic construct
EYDMH

SEQ ID NO: 290 - protein, 17 aa, synthetic construct
TISSSGSTIYYADSVKG

SEQ ID NO: 291 - protein, 17 aa, synthetic construct
SITSSGSTIYYADSVKG

SEQ ID NO: 292 - protein, 17 aa, synthetic construct
SISTSGSTIYYADSVKG

SEQ ID NO: 293 - protein, 17 aa, synthetic construct
SISSTGSTIYYADSVKG

SEQ ID NO: 294 - protein, 17 aa, synthetic construct
SISSSGSTIYYAESVKG

SEQ ID NO: 295 - protein, 17 aa, synthetic construct
SISSSGSTIYYADTVKG

SEQ ID NO: 296 - protein, 17 aa, synthetic construct
SISSSGSTIYYADSVRG

SEQ ID NO: 297 - protein, 17 aa, synthetic construct
SISSSGSTIYYADSVRA

SEQ ID NO: 298 - protein, 17 aa, synthetic construct
SISSSGSTIYYADSVKA

SEQ ID NO: 299 - protein, 17 aa, synthetic construct
SLSSSGSTIYYADSVKG

SEQ ID NO: 300 - protein, 10 aa, synthetic construct
AVVGKKYFQH

SEQ ID NO: 301 - protein, 10 aa, synthetic construct
AVVGRKYFNH

SEQ ID NO: 302 - protein, 10 aa, synthetic construct
AVVGKKYFNH

SEQ ID NO: 303 - protein, 10 aa, synthetic construct
AVVARKYFQH

SEQ ID NO: 304 - protein, 10 aa, synthetic construct
GVVGRKYFQH

SEQ ID NO: 305 - protein, 10 aa, synthetic construct
AVAGRKYFQH

SEQ ID NO: 306 - protein, 10 aa, synthetic construct
AAVGRKYFQH

SEQ ID NO: 307 - protein, 10 aa, synthetic construct
AVVGKKYFNH

SEQ ID NO: 308 - protein, 10 aa, synthetic construct
GVVGKKYFQH

SEQ ID NO: 309 - protein, 10 aa, synthetic construct
ALVGRRYFQH

SEQ ID NO: 310 - protein, 5 aa, synthetic construct
TYEMS

SEQ ID NO: 311 - protein, 5 aa, synthetic construct
SYEMT

SEQ ID NO: 312 - protein, 5 aa, synthetic construct
SYDMS

SEQ ID NO: 313 - protein, 5 aa, synthetic construct
SYEMH

SEQ ID NO: 314 - protein, 5 aa, synthetic construct
SYEMA

SEQ ID NO: 315 - protein, 5 aa, synthetic construct
SYEMN

SEQ ID NO: 316 - protein, 5 aa, synthetic construct
TYEMH

SEQ ID NO: 317 - protein, 5 aa, synthetic construct
TYEMN

SEQ ID NO: 318 - protein, 5 aa, synthetic construct
SYDMA

SEQ ID NO: 319 - protein, 5 aa, synthetic construct
SYDMH

SEQ ID NO: 320 - protein, 17 aa, synthetic construct
YITSSGSTIYYADSVKG

SEQ ID NO: 321 - protein, 17 aa, synthetic construct
YISTSGSTIYYADSVKG

SEQ ID NO: 322 - protein, 17 aa, synthetic construct
YISSTGSTIYYADSVKG

SEQ ID NO: 323 - protein, 17 aa, synthetic construct
YISSSGTTIYYADSVKG

SEQ ID NO: 324 - protein, 17 aa, synthetic construct
YISSSGSSIYYADSVKG

SEQ ID NO: 325 - protein, 17 aa, synthetic construct
YISSSGSTIYYAESVKG

SEQ ID NO: 326 - protein, 17 aa, synthetic construct
YISSSGSTIYYADTVKG

SEQ ID NO: 327 - protein, 17 aa, synthetic construct
YISSSGSTIYYADSVRG

SEQ ID NO: 328 - protein, 17 aa, synthetic construct
YISSSGSTIYYADSVKA

SEQ ID NO: 329 - protein, 17 aa, synthetic construct
YISSSGSTIYYADSAKG

SEQ ID NO: 330 - protein, 9 aa, synthetic construct
NPAPGTADN

SEQ ID NO: 331 - protein, 9 aa, synthetic construct
NPAPGTAEQ

SEQ ID NO: 332 - protein, 9 aa, synthetic construct
NPAPGSADQ

SEQ ID NO: 333 - protein, 9 aa, synthetic construct
QPAPGTADQ

SEQ ID NO: 334 - protein, 9 aa, synthetic construct
NPAPGTADH

SEQ ID NO: 335 - protein, 9 aa, synthetic construct
QPAPGSADQ

SEQ ID NO: 336 - protein, 9 aa, synthetic construct
QPAPGTADN

SEQ ID NO: 337 - protein, 9 aa, synthetic construct
QPAPGSADH

SEQ ID NO: 338 - protein, 9 aa, synthetic construct
NPAPGTAEH

SEQ ID NO: 339 - protein, 9 aa, synthetic construct
QPAPGSAEN

SEQ ID NO: 340 - protein, 5 aa, synthetic construct
TYEMS

SEQ ID NO: 341 - protein, 5 aa, synthetic construct
SYEMT

SEQ ID NO: 342 - protein, 5 aa, synthetic construct
SYDMS

SEQ ID NO: 343 - protein, 5 aa, synthetic construct
SYEMH

SEQ ID NO: 344 - protein, 5 aa, synthetic construct
SYEMA

SEQ ID NO: 345 - protein, 5 aa, synthetic construct
SYEMN

SEQ ID NO: 346 - protein, 5 aa, synthetic construct
TYEMH

SEQ ID NO: 347 - protein, 5 aa, synthetic construct
TYEMN

SEQ ID NO: 348 - protein, 5 aa, synthetic construct
SYDMA

SEQ ID NO: 349 - protein, 5 aa, synthetic construct
SYDMH

SEQ ID NO: 350 - protein, 17 aa, synthetic construct
YITSSGSTIYYADSVKG

SEQ ID NO: 351 - protein, 17 aa, synthetic construct
YISTSGSTIYYADSVKG

SEQ ID NO: 352 - protein, 17 aa, synthetic construct
YISSTGSTIYYADSVKG

SEQ ID NO: 353 - protein, 17 aa, synthetic construct
YISSSGTTIYYADSVKG

SEQ ID NO: 354 - protein, 17 aa, synthetic construct
YISSSGSSIYYADSVKG

SEQ ID NO: 355 - protein, 17 aa, synthetic construct
YISSSGSTIYYAESVKG

SEQ ID NO: 356 - protein, 17 aa, synthetic construct
YISSSGSTIYYADTVKG

SEQ ID NO: 357 - protein, 17 aa, synthetic construct
YISSSGSTIYYADSVRG

SEQ ID NO: 358 - protein, 17 aa, synthetic construct
YISSSGSTIYYADSVKA

SEQ ID NO: 359 - protein, 17 aa, synthetic construct
YISSSGSTIYYADSAKG

SEQ ID NO: 360 - protein, 9 aa, synthetic construct
NPAPGTVDN

SEQ ID NO: 361 - protein, 9 aa, synthetic construct
NPAPGTVEQ

SEQ ID NO: 362 - protein, 9 aa, synthetic construct
NPAPGSVDQ

SEQ ID NO: 363 - protein, 9 aa, synthetic construct
QPAPGTVDQ

SEQ ID NO: 364 - protein, 9 aa, synthetic construct
NPAPGTVDH

SEQ ID NO: 365 - protein, 9 aa, synthetic construct
QPAPGSVDQ

SEQ ID NO: 366 - protein, 9 aa, synthetic construct
QPAPGTVDN

SEQ ID NO: 367 - protein, 9 aa, synthetic construct
QPAPGSADH

SEQ ID NO: 368 - protein, 9 aa, synthetic construct
NPAPGTAEH

SEQ ID NO: 369 - protein, 9 aa, synthetic construct
QPAPGSAEN

SEQ ID NO: 370 - protein, 15 aa, synthetic construct
GGGGSGGGGSGGGGS

SEQ ID NO: 371 - protein, 15 aa, synthetic construct
GSGSSGSGSSGSGSS

SEQ ID NO: 372 - protein, 6 aa, synthetic construct
GSGSSG

SEQ ID NO: 373 - protein, 17 aa, synthetic construct
MNWVRQAPGKGLEWVSA

SEQ ID NO: 374 - DNA, 333 bp, synthetic construct; identical to SEQ ID NO: 151
  source 1..333: mol_type = other DNA

SEQ ID NO: 375 - protein, 25 aa, synthetic construct
VPVLTQPHSVSESPGKTVTISCTRS

SEQ ID NO: 376 - protein, 17 aa, synthetic construct
VQWYQQRPGSSPTTVIY

SEQ ID NO: 377 - protein, 38 aa, synthetic construct
QRPSGVPDRFSGSIDMSSNSASLTISGLRTEDEGDYYC

SEQ ID NO: 378 - protein, 11 aa, synthetic construct
FGGGTQLTVLG

SEQ ID NO: 379 - protein, 20 aa, synthetic construct
GGGGSGGGGSGGGGSGGGGS